(19) United States
(12) Patent Application Publication
(10) Pub. No.: US 2005/0124800 A1
(43) Pub. Date: Jun. 9, 2005

(54) Mammalian migration inducting gene and methods for detection and inhibition of migrating tumor cells

(76) Inventor: Suzanne J. Lindsey, Amarillo, TX (US)

Correspondence Address: Michael L. Goldman;Nixon Peabody, Clinton Square, PO Box 31051, Rochester, NY, 14603, US

(21) Appl. No.: 10/502,163

(22) PCT Filed: Jan. 23, 2003
(86) PCT No.: PCT/US03/02047

Related U.S. Application Data
(60) Provisional application No. 60/351,073, filed on Jan. 23, 2002.

(51) Int. Cl. C12Q 1/68; C07H 21/04; A61K 48/00

(52) U.S. Cl. 536/23.2; 435/6; 435/69.1; 435/320.1; 435/325; 514/44; 530/350

(57) ABSTRACT

The present invention relates to isolated nucleic acid molecules conferring on mammalian carcinoma cells an ability to undergo cell migration. Recombinant DNA expression systems and host cells containing the subject nucleic acid molecule, as well as antisense oligonucleotides, are also described. Also disclosed are methods of inhibiting expresssion of the subject nucleic acid molecule, inhibiting production of the encoded protein or polypeptide, inhibiting metastasis of a carcinoma cell in a subject (including in humans), inhibiting migration of carcinoma cell in a subject, detecting the presence of a migrating carcinoma cell in a sample of a subject's tissue or body fluids, and inhibiting the migration of a placental cell into the blood stream of a female mammal.

This application claims the benefit of U.S. Provisional Patent Application Ser. No. 60/351,073, filed Jan. 23, 2002.

### FIELD OF THE INVENTION

The present invention relates to isolated nucleic acid molecules conferring on a mammalian carcinoma cell an ability to undergo cell migration, and methods for detecting and inhibiting the migration of tumor and placental cells.

### BACKGROUND OF THE INVENTION

Metastasis relies on the mechanisms of cell scattering, breakdown of extracellular matrix, migration, and mitosis. Interactions between the cells of the primary tumor and the surrounding stroma are a primary research focus for the study of metastasis. One product of the stroma, hepatocyte growth factor (“HGF”), also known as scatter factor (“SF”), and its receptor, the protooncogene c-Met (also referred to as “Met”), are upregulated in most metastatic cancers and are indicators of a poor prognosis (Jian et al., “Hepatocyte Growth Factor/Scatter Factor, its Molecular, Cellular and Clinical implications in Cancer,” Critical Reviews in Oncology/Hematology 29:209-248 (1999); Vande Woude et al., “Met-HGF/SF: Tumorigenesis, Invasion and Metastasis,” Ciba Foundation Symposium 212:119-130 (1997)).

Cell migration during metastasis relies on interactions between growth factors, extracellular matrix, and cell membrane receptors. Therefore, potential cancer cell-specific targets include molecules involved in the processes of uncontrolled cell proliferation, migration of tumor cells, and new blood supply to the tumor. HGF and c-Met are involved in all three of these processes. HGF and Met are expressed in normal cells and therefore are not good candidates for cancer cell-specific targets. Because cancer cell migration requires HGF/Met-induced de novo transcription, cancer-specific genes could be induced by activation of the HGF/Met pathway. Until recently, the gene(s) induced by HGF during cell migration were unknown. However, the relationship between HGF and c-Met and their involvement with tumorigenesis and metastasis have been studied and reported.

For example, it has been reported that HGF treatment of carcinoma cell lines that express c-Met causes increased migration and invasion (Vande Woude et al., “Met-HGF/SF: Tumorigenesis, Invasion and Metastasis,” Ciba Foundation Symposium 212:119-130 (1997); Bae-Jump et al., “Hepatocyte Growth Factor (HGF) Induces Invasion of Endometrial Carcinoma Cell Lines In Vitro,” Gynecologic Oncology 73:265-272 (1999); Birchmeier et al., “Role of HGF/SF and c-Met in Morphogenesis and Metastasis of Epithelial Cells,” Ciba Foundation Symposium 212:230-246 (1997); Trusolino et al., “A Signaling Adapter Function for a6b₄ Integrin in the Control of HGF-Dependent Invasive Growth,” Cell 107:643-654 (2001)). Various inhibitors of HGF binding to c-Met such as neutralizing antibodies, and truncated forms of HGF, have been shown to inhibit migration in vitro and metastasis in mouse tumor models (Chan et al., “Identification of a Competitive HGF Antagonist Encoded by an Alternative Transcript,” Science 251:802-804 (1991); Lokker et al., “Generation and Characterization of a Competitive Antagonist of Human Hepatocyte Growth Factor, HGF/NK1,” Journal of Biological Chemistry 268:17145-17150 (1993); Cao et al., “Neutralizing Monoclonal Antibodies to Hepatocyte Growth Factor/Scatter Factor (HGF/SF Display Antitumor Activity in Animal Models,” Proceedings of the National Academy of Science 98:443-7448 (2001)).

While agents used to inhibit binding of HGF to c-Met inhibit cell migration, this inhibition is not cancer cell-specific. For example, HGF stimulates other normal physiological events such as wound healing (Ferrara, N., “Vascular Endothelial Growth Factor and the Regulation of Angiogenesis,” Recent Progress in Hormone Research 55:15-36 (2000); Imanishi et al., “Growth Factors: Importance in Wound Healing and Maintenance of Transparency of the Cornea,” Progress in Retinal & Eye Research 19(1):113-129 (2000)), B cell migration (van der Voort et al., “Paracrine Regulation of Germinal Center B Cell Adhesion Through the c-Met-Hepatocyte Growth Factor/Scatter Factor Pathway,” Journal of Experimental Medicine 185:2121-2131 (1997)), small intestine re-epithelialization (Watanabe et al., “Epithelial-Mesenchymal Interaction in Gastric Mucosal Restoration,” Journal of Gastroenterology 35:65-68 (2000)), maintenance of the cornea in the eye (Imanishi et al., “Growth Factors: Importance in Wound Healing and Maintenance of Transparency of the Cornea,” Progress in Retinal & Eye Research 19(1):113-129 (2000)) and bone remodeling (Fuller et al., “The Effect of Hepatocyte Growth Factor on the Behaviour of Osteoclasts,” Biochem. Biophys. Res. Commun. 212:334-340 (1995); Grano et al., “Hepatocyte Growth Factor is a Coupling Factor for Osteoclasts and Osteoblasts In Vitro,” Proceedings of the National Academy of Science 93:7644-7648 (1996)). Therefore, because expression of HGF or c-Met is not cancer cell specific, targeting them in vivo may cause serious side effects during anti-cancer treatment.

HGF is regarded as a pleiotropic factor. Through binding c-Met, HGF causes cell proliferation, angiogenesis, morphogenesis, and migration (Jiang et al., “Hepatocyte Growth Factor/Scatter Factor, a Cytokine Playing Multiple and Converse Roles,” Histology & Histopathology 12:537-555 (1997)). Two known determinants of the various effects caused by HGF stimulating c-Met are the differentiation state of the stimulated cell (Birchmeier et al., “Role of HGF/SF and c-Met in Morphogenesis and Metastasis of Epithelial Cells,” Ciba Foundation Symposium 212:230-246 (1997); Fehlner-Gardiner et al., “Characterization of a Functional Relationship Between Hepatocyte Growth Factor and Mouse Bone Marrow-Derived Mast Cells,” Differentiation 65:27-42 (1999); Byers et al., “Breast Carcinoma: A Collective Disorder,” Breast Cancer Research & Treatment 31:203-215 (1994)), and signaling through integrin activation (Beviglia et al., “HGF Induces FAK Activation and Integrin-Mediated Adhesion in MTLn3 Breast Carcinoma Cells,” International Journal of Cancer 83:640-649 (1999); Trusolino et al., “HGF/Scatter Factor Selectively Promotes Cell Invasion by Increasing Integrin Avidity,” FASED Journal 14:1629-1640 (2000)). De novo transcription is required for HGF induction of migration (Birchmeier et al., “Role of HGF/SF and c-Met in Morphogenesis and Metastasis of Epithelial Cells,” Ciba Foundation Symposium 212:230-246 (1997); Rosen et al., “Regulation of Angiogenesis by Scatter Factor,” Experientia Supplementum 79:193-208 (1997)).

Further, HGF-induced transcripts expressed early in the migration signaling pathway have not been isolated. Prior investigations identified HGF induction of metalloproteinase (Dunsmore et al., “Mechanisms of Hepatocyte Growth Factor Stimulation of Keratinocyte Metalloproteinase Production,” Journal of Biological Chemistry 271:24576-24582 (1996)), osteopontin (Tuck et al., “Osteopontin-Induced, Integrin-Dependent Migration of Human Mammary Epithelial Cells Involves Activation of the Hepatocyte Growth Factor Receptor,” Journal of Cellular Biochemistry 78:465-475 (2000)), or integrin expression (Liang et al., “Sustained Activation of Extracellular Signal-Regulated Kinase Stimulated by Hepatocyte Growth Factor Leads to Integrin Alpha 2 Expression that is Involved in Cell Scattering,” Journal of Biological Chemistry 276:1146-21152 (2001)) 24 to 48 hours after treatment. It is not surprising that these genes are not cancer cell-specific due to the late stage of HGF-signaling used during their isolation. HGF induction of immediate early events prior to migration, such as breakdown of E-cadherin connections, have also been determined (Miura et al., “Effects of Hepatocyte Growth Factor on E-Cadherin-Mediated Cell-Cell Adhesion in DU145 Prostate Cancer Cells,” Urology 58:1064-1069 (2001); Davies et al., “Matrilysin Mediates Extracellular Cleavage of E-Cadherin From Prostate Cancer Cells: A Key Mechanism in Hepatocyte Growth Factor/Scatter Factor-Induced Cell-Cell Dissociation and in vitro Invasion,” Clinical Cancer Research 7:3289-3297 (2001)).

Previous work has shown that genes expressed early in signal transduction pathways tend to be expressed in a cell-specific manner (Lindsey et al., “Pem: A Testosterone- and LH-Regulated Homeobox Gene Expressed in Mouse Sertoli Cells and Epididymis,” Developmental Biology 179:471-484 (1996); Witzenbichler et al., “Regulation of Smooth Muscle Cell Migration and Integrin Expression by the Gax Transcription Factor,” Journal of Clinical Investigation 104:1469-1480 (1999)). It is therefore conceivable that HGF induces cancer cell-specific gene transcription due to their dedifferentiated state and the fact that early-transcribed genes can be cell specific in their expression pattern. These cancer cell-specific, HGF-induced transcripts may be used as targets to inhibit migration and invasion.

It is also known that HGF stimulation of c-Met on epithelial cells induces specific signaling cascades. These signaling events cause epithelial cells to scatter, produce metalloproteinases, and migrate. It has been shown that de novo transcription is required for HGF-induced migration (Rosen et al., “Studies On the Mechanism of Scatter Factor. Effects of Agents That Modulate Intracellular Signal Transduction, Maromolecule Synthesis and Cytoskeleton Assembly,” Journal of Cell Science 96:639-649 (1990)); a key event required for metastasis. However, the newly transcribed genes have not previously been identified. Furthermore, it is clear that various signal transduction pathways are involved with HGF responsiveness (Birchmeier et al., “Role of HGF/SF and c-Met in Morphogenesis and Metastasis of Epithelial Cells,” Ciba Foundation Symposium 212:230-246 (1997)), but it is unclear why one cell type responds with proliferation and another cell type responds with migration. Since HGF and c-Met are upregulated at the invading edge of the tumor in almost every metastatic cancer (Jian et al., “Hepatocyte Growth Factor/Scatter Factor, its Molecular, Cellular and Clinical Implications in Cancer,” Critical Reviews in Oncology/Hematology 29:209-248 (1999); Vande Woude et al., “Met-HGF/SF: Tumorigenesis, Invasion and Metastasis,” Ciba Foundation Symposium 212:119-130 (1997)), it is important to understand how HGF induces cell migration. The problem is that the genes induced by HGF are not known and the interaction of molecules required for this induction is not known. There is the promise that, in metastatic cancers that are associated with HGF/Met expression, carcinoma cell migration could be inhibited by blocling expression of HGF induced, cancer cell-specific migration inducing genes. New approaches of this kind would be expected to augment the efficacy of traditional therapies significantly, especially post surgery when undetected migrating cancer cells could be inhibited.

As previously mentioned, HGF and its tyrosine kinase receptor, Met, play an important role in cancer progression. In a number of malignant tumors, HGF and Met are mutated, amplified or overexpressed (Vande Woude et al., “Met-HGF/SF: Tumorigenesis, Invasion and Metastasis,” Ciba Foundation Symposium 212:119-130 (1997); To et al., “The Roles of Hepatocyte Growth Factor/Scatter Factor and Met Receptor in Human Cancers,” Oncology Reports 5:1013-1024 (1998)). Transgenic mice overexpressing HGF exhibit multiple sites of histologically distinct tumors of mesenchymal and epithelial origins (Takayama et al., “Diverse Tumorigenesis Associated with Aberrant Development in Mice Overexpressing Hepatocyte Growth Factor/Scatter Factor,” Proceedings of the National Academy of Science 94:701-706 (1997)). HGF/Met increases B-lymphoma cell migration (mediated by alpha4 beta1 and alpha5 beta1 integrins) by six fold (Weimar et al., “Hepatocyte Growth Factor/Scatter Factor Promotes Adhesion of Lymphoma Cells to Extracellular Matrix Molecules via Alpha 4 Beta 1 and Alpha 5 Beta 1 Integrins,” Blood 89:990-1000 (1997)). In addition, HGF/Met induces focal degradation of extracellular matrix (a mechanism required for invasion) by activating urokinase type 1 plasminogen activator (Rosen et al., “Regulation of Angiogenesis by Scatter Factor,” Experientia Supplementum 79:193-208 (1997)). Therefore, HGF/Met signaling pathways and the resultant gene regulation have been the focus of many researchers.

It is well documented in the literature that HGF/Met induces tumorigenesis and metastasis. However, little is known about the signal transduction pathways by which HGF/Met exerts these effects. Part of the HGF/Met signaling cascade has been defined. Weidner et al. have defined a unique binding site on Met for Gab-1 (Weidner et al., “Interaction Between Gab1 and the c-Met Receptor Tyrosine Kinase is Responsible for Epithelial Morphogenesis,” Nature 384:173-176 (1996)), a Grb2-binding protein. Gab-1 and Grb2 act in signaling pathways downstream of tyrosine kinase receptors including the receptors for nerve growth factor (Holgado-Madruga et al., “Grb2-Associated Binder-1 Mediates Phosphatidylinositol 3-Kinase Activation and the Promotion of Cell Survival by Nerve Growth Factor,” Proceedings of the National Academy of Science 94:12419-12424 (1997)), epidermal growth factor and insulin (Holgado-Madruga et al., “A Grb2-Associated Docking Protein in EGF- and Insulin-Receptor Signaling,” Nature 379:560-564 (1996)). Boccaccio et al. have shown that the phosphatidylinositol-3-inositol-3 Kinase (PI3K) and rac pathway, the ras-MAP kinase cascade, and the STAT pathway, control HGF/Met induced migration, mitosis, and tubulogenesis, respectively (Boccaccio et al., “Induction of Epithelial Tubules by Growth Factor HGF Depends on the STAT Pathway,” Nature 391:285-288 (1998)).

In hepatoma cells, HGF activates Met by phosphorylating the STAT3/APRF transcription factor (Schaper et al., “Hepatocyte Growth Factor/Scatter Factor (HGF/SF) Signals via the STAT3/APRF Transcription Factor in Human Hepatoma Cells and Hepatocytes,” FEBS Letters 405:99-103 (1997)), a factor known to be induced in other tissues by various cytokines during an acute phase response (Akira, “IL-6-Regulated Transcription Factors,” International Journal of Biochemistry & Cell Biology 29:1401-1418 (1997)). Thus far, none of the HGF/Met signal transduction pathways have been found to be cancer cell-specific. Furthermore, while several genes induced by HGF/Met have been identified (e.g., c/ebp beta, plasminogen activator inhibitor type 1, tissue factor, CD44 and ETS1 (Shen et al., “Transcriptional Induction of the agp/ebp (c/ebp beta) Gene by Hepatocyte Growth Factor,” DNA Cell Biology 16:703-711 (1997); Wojta et al., “Hepatocyte Growth Factor Stimulates Expression of Plasminogen Activator Inhibitor Type 1 and Tissue Factor in HepG2 Cells,” Blood 84:151-157 (1994); Fafeur et al., “The ETS1 Transcription Factor is Expressed During Epithelial-Mesenchymal Transitions in the Chick Embryo and is Activated in Scatter Factor-Stimulated MDCK Epithelial Cells,” Cell Growth and Differentiation 8:655-665 (1997); Hiscox et al., “Regulation of Endothelial CD44 Expression and Endothelium-Tumour Cell Interactions by Hepatocyte Growth Factor/Scatter Factor,” Biochemistry and Biophysiology Research Communications 233:1-5 (1997)), so far none are cancer cell-specific. Therefore, HGF and Met do not make good cancer cell-specific therapeutic targets, because many tissues rely on HGF/Met mediated gene regulation for their normal function.

Normal adult skeletal muscle expresses HGF. Activated satellite cells involved in muscle repair express both HGF and Met in an autocrine fashion. Both in vitro and in vivo evidence indicates that HGF activates satellite cells to divide in skeletal muscle (Tatsumi et al., “HGF/SF is Present in Normal Adult Skeletal Muscle and is Capable of Activating Satellite Cells,” Developmental Biology 194:114-128 (1998)). Human bone marrow stromal cells produce HGF that promotes proliferation, adhesion and survival of hematopoietic, CD34+ progenitor cells (Weimar et al., “Hepatocyte Growth Factor/Scatter Factor (HGF/SF) is Produced by Human Bone Marrow Stromal Cells and Promotes Proliferation, Adhesion and Survival of Human Hematopoietic Progenitor Cells (CD34+),” Experimental Hematology 26:885-894 (1998)). HGF stimulates chemotactic migration and DNA replication in Met positive, primary osteoclasts;
osteoclasts are then stimulated to produce HGF, which acts in a paracrine manner on osteoblasts to produce collagen and MMP-2 and -9. These data suggest a role for HGF/Met in bone formation (Grano et al., “Hepatocyte Growth Factor is a Coupling Factor for Osteoclasts and Osteoblasts In vitro,” Proceedings of the National Academy of Science 93:7644-7648 (1996)).

Another normal autocrine example of HGF/Met expression is in axons where it is necessary for optimal axon growth (Yang et al., “Autocrine Hepatocyte Growth Factor Provides a Local Mechanism for Promoting Axonal Growth,” Journal of Neuroscience 18:8369-8381 (1998)). In the immune system, T cell-dependent humoral immune responses require activation and migration of naïve B cells. Activated tonsil B cells transiently express Met and migrate in response to tonsilar stromal cell production of HGF (van der Voort et al., “Paracrine Regulation of Germinal Center B Cell Adhesion Through the c-Met-Heaptocyte Growth Factor/Scatter Factor Pathway,” Journal of Experimental Medicine 185:2121-2131(1997)). Furthermore, Adams et al. demonstrated that HGF induces migration of human memory T cells (Adams et al., “Hepatocyte Growth Factor and Macrophage Inflammatory Protein 1 Beta: Structurally Distinct Cytokines that Induce Rapid Cytoskeletal Changes and Subset-Preferential Migration in T Cells,” Proceedings of the National Academy of Science 91:7144-7148 (1994)).

In addition to the critical functions of repair, immune response, hematopoiesis, and bone formation, this paracrine/autocrine HGF/Met system is also expressed in other tissues. HGF causes lumen formation and stimulates migration of endometrial epithelial cells in vitro (Sugawara et al., “Hepatocyte Growth Factor Stimulated Proliferation, Migration, and Lumen Formation of Human Endometrial Epithelial Cells In vitro,” Biology of Reproduction 57:936-942 (1997)). In vivo, HGF and Met are expressed by the stroma and epithelial cells respectively of the endometrium that is consistent with remodeling the glandular epithelium and migration of these epithelial cells during the early proliferative phase of the menstrual cycle. In rat ovary, both theca-interstitial cells and granulosa cells express HGF which, in turn, inhibits luteinizing hormone-stimulated androgen production, suggesting HGF has a role in folliculogenesis (Zachow et al., “Hepatocyte Growth Factor Regulates Ovarian Theca-Interstitial Cell Differentiation and Androgen Production,” Endocrinology 138:691-697 (1997)).

Cell motility requires adhesion receptor expression. For example, metastasis of multiple tumor types requires ligation of both αvβ5 integrin and cytokine receptor (Brooks et al., “Insulin-Like Growth Factor Receptor Cooperates with Integrin Alpha v Beta 5 to Promote Tumor Cell Dissemination In Vivo,” Journal of Clinical Investigation 99:1390-1398 (1997)). In addition, HGF activates integrins. Specifically, HGF has been shown to induce integrin-mediated adhesion in breast carcinoma cells (Beviglia et al., “HGF Induces FAK Activation and Integrin-Mediated Adhesion in MTLn3 Breast Carcinoma Cells,” International Journal of Cancer 83:640-649 (1999)), colon carcinoma cells (Fujisaki et al., “CD44 Stimulation Induces Integrin-Mediated Adhesion of Colon Cancer Cell Lines to Endothelial Cells by Up-Regulation of Integrins, c-Met and Activation of Integrins,” Cancer Research 59:4427-4434 (1999)) and thyroid papillary carcinoma cells (Trusolino et al., “Growth Factor-Dependent Activation of αvβ3 Integrin in Normal Epithelial Cells: Implications for Tumor Invasion,” Journal of Cell Biology 142:1145-1156 (1998)). However, specific genes expressed as a result of this signaling are unknown.

Integrins are a class of genes that change expression based on the cellular differentiation state and play a key role in cell migration. Integrins transmit extracellular signals into the cell by binding extracellular matrix ligands. They can also transmit signals from within the cell to the outside by intracellular modulation of extracellular binding activity (Giancotti et al., “Integrin Signaling,” Science 285:1028-1032 (1999)). Laminin and β1 integrin has been shown to be important for tubulogenesis induced by HGF (Klinowska et al., “Laminin and β1 Integrins are Crucial for Normal Mammary Gland Development in the Mouse,” Developmental Biology 215:13-32(1999)). Integrins have been shown to play a role in metastasis and are regulated by growth factors (Matsumoto et al., “Growth Factor Regulation of Integrin-Mediated Cell Motility,” Cancer and Metastasis Reviews 14:205-207 (1995)). Tyrosine kinase receptor activation and induction of MCF7 breast carcinoma and FG pancreatic carcinoma cell migration is dependent upon αvβ5 binding (Klemke et al., “Receptor Tyrosine Kinase Signaling Required for Integrin Alpha v Beta 5-Directed Cell Motility but not Adhesion on Vitronectin,” Journal of Cell Biology 127:859-866 (1994)). However, the mechanisms underlying the cross-talk between tyrosine kinase receptors and αvβ5 activation and the resulting carcinoma cell migration are not fully understood.

HGF activates integrins. Specifically, HGF has been shown to induce integrin-mediated adhesion in breast carcinoma cells (Beviglia et al., “HGF Induces FAK Activation and Integrin-Mediated Adhesion in MTLn3 Breast Carcinoma Cells,” International Journal of Cancer 83:640-649 (1999)), colon carcinoma cells (Fujisaki et al., “CD44 Stimulation Induces Integrin-Mediated Adhesion of Colon Cancer Cell Lines to Endothelial Cells by Up-Regulation of Integrins, c-Met and Activation of Integrins,” Cancer Research 59:44274434 (1999)) and thyroid papillary carcinoma cells (Trusolino et al., “Growth Factor-Dependent Activation of αvβ3 Integrin In Normal Epithelial Cells: Implications for Tumor Invasion,” Journal of Cell Biology 142:1145-1156 (1998)). Integrins can bind to the RGD (arginine-glycine-aspartate) motif in extracellular matrix proteins. For example, the chemotactic factor, osteopontin, binds through its RGD site specifically to αvβ3, αvβ1, and αvβ5 integrins (Denhardt et al., “Osteopontin: a protein with diverse functions,” FASED Journal 7:1475-1482 (1993)). Also, Transforming Growth Factor-β Latent Associated Protein binds to αvβ1 (Munger et al., “Interactions Between Growth Factors and Integrins: Latent Forms of Transforming Growth Factor-b Are Ligands for the Integrin αvβ1,” Molecular Biology of the Cell 9:2627-2638 (1998)) and αvβ6 (Munger et al., “The Integrin Alpha v Beta 6 Binds and Activates Latent TGF Beta 1: A Mechanism for Regulating Pulmonary Inflammation and Fibrosis,” Cell 96:319-328 (1999)). Even though an RGD site is known to exist in HGF at amino acids 556-558 (Seki et al., “Isolation and Expression of cDNA for Different Forms of Hepatocyte Growth Factor from Human Leukocyte,” Biochemical and Biophysical Research Communications 172:321-327 (1990)), its integrin binding capability has not been reported.

HGF has been shown to regulate genes in a cell specific manner. The homeobox transcription factor, Gax, is specifically expressed in normal smooth muscle cells of the heart, lung, and arteries. Gax expression downregulates αvβ3 and αvβ5 expression during the migration of smooth muscle cells. HGF inhibits expression of Gax and, therefore, is a cell specific gene regulated by HGF (Witzenbichler et al., “Regulation of Smooth Muscle Cell Migration and Integrin Expression by the Gax Transcription Factor,” Journal of Clinical Investigation 104:1469-1480 (1999)).

Cancer cell-specific targets are needed because current therapies for cancer tend to create new and additional problems for the patient Radiation has been shown to cause mutations that can lead to different types of cancer in the future. Chemotherapies cause toxicity to normal tissues of the body. Vital functions, such as immune protection, which require cell division, are inhibited, thereby making the patient more susceptible to other diseases. The lethal part of cancer is migration of cancer cells from the primary tumor to other organs of the body also known as metastasis. Targeting cancer cell-specific genes contributing to metastasis would be highly beneficial. HGF and its protooncogene receptor, c-Met, have repeatedly been shown to cause cancer cells to migrate but is also involved in normal cellular functions. In addition, new gene expression is required for HGF-induced carcinoma cell migration.

Existing methods of treating cancer, such as chemotherapy and radiation, cause many unwanted side effects, including secondary cancers, because they also target normal cells. Therefore, cancer cell-specific treatments are one of the major goals of cancer research. In recent years, HGF/Met signaling mechanisms have been partially described. However, carcinoma cell-specific genes induced by this signaling pathway have not been identified or are only partially characterized. Thus, defining the molecular mechanisms of HGF-induced gene expression and Met signaling during cancer cell migration could lead to the development of novel therapeutics that are cancer cell specific.

The present invention is directed to overcoming these and other deficiencies in the art.

### SUMMARY OF THE INVENTION

The present invention relates to an isolated nucleic acid molecule conferring on a mammalian carcinoma cell an ability to undergo cell migration. In one aspect of the present invention, the nucleic acid molecule is a mammalian migration inducting gene, such as Mig-7. The isolated nucleic acid molecule may have a nucleotide sequence corresponding to SEQ ID NO:1 or SEQ ID NO:2, a nucleotide sequence that is 99 percent homologous to SEQ ID NO:1 or SEQ ID NO:2, or a nucleotide sequence of at least 18 contiguous nucleic acid residues that hybridize to either SEQ ID NO:1 or SEQ ID NO:2 under any of the following stringent conditions: (a) 6×SSC at 68° C.; (b) 5×SSC and 50% formamide 37° C.; or (c) 2×SSC and 40% formamide at 40° C. Another aspect of the present invention involves an isolated nucleic acid molecule that encodes a protein or polypeptide comprising an amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, or SEQ ID NO:30.

The present invention also relates to a recombinant DNA expression system and a host cell incorporating an isolated nucleic acid molecule conferring on a mammalian carcinoma cell an ability to undergo cell migration.

The present invention also relates to an antisense oligonucleotide of at least 8 contiguous nucleic acid residues targeted to a nucleic acid molecule conferring on a mammalian carcinoma cell an ability to undergo cell migration. The antisense oligonucleotide may hybridize to an isolated nucleic acid molecule that: codes for a mammalian migration inducting gene (e.g., Mig-7), has a nucleotide sequence of SEQ ID NO:1 or SEQ ID NO:2, has a nucleotide sequence that is 99 percent homologous to SEQ ID NO:1 or SEQ ID NO:2, or has a nucleotide sequence of at least 18 contiguous nucleic acid residues that hybridize to SEQ ID NO:1 or SEQ ID NO:2 under the following stringent conditions: (a) 6×SSC at 68° C.; (b) 5×SSC and 50% formamide 37° C.; or (c) 2×SSC and 40% formamide at 40° C. The antisense oligonucleotide may hybridize to nucleotides 275 to 292 or nucleotides 324 to 343 of SEQ ID NO:1, or to nucleotides 760 to 777 or nucleotides 809 to 828 of SEQ ID NO:2.

The present invention also relates to a method for inhibiting expression, in a subject, of a nucleic acid molecule conferring on a human carcinoma cell an ability to undergo cell migration. This method involves administering to the subject an inhibitor capable of blocking binding of a growth factor to at least one receptor for the growth factor under conditions effective to inhibit the expression of the nucleic acid molecule.

The present invention also relates to a method for inhibiting production, in a subject, of a protein or polypeptide encoded by a nucleic acid molecule conferring on a carcinoma cell an ability to undergo cell migration. This method involves administering to the subject the antisense oligonucleotide of the present invention, which is complementary to a target portion of the nucleic acid molecule, under conditions effective to inhibit production of the protein or polypeptide.

The present invention also relates to a method for inhibiting metastasis of a carcinoma cell in a subject. This method involves administering to the subject the antisense oligonucleotide of the present invention, which is complementary to a target portion of a nucleic acid molecule conferring on a carcinoma cell an ability, in vivo, to undergo cell migration under conditions effective to inhibit metastasis of the carcinoma cell.

The present invention also relates to a method for inhibiting metastasis of a carcinoma cell in a human subject. This method involves administering to the subject an inhibitor capable of blocking the binding of a growth factor to at least one receptor for the growth factor under conditions effective to inhibit metastasis of the carcinoma cell.

The present invention also involves a method for inhibiting migration of a carcinoma cell in a subject. This method involves administering to the subject the antisense oligonucleotide of the present invention, which is complementary to a target portion of a nucleic acid molecule conferring on a carcinoma cell an ability, in vivo, to undergo cell migration, under conditions effective to inhibit migration of the carcinoma cell.

The present invention also relates to a method for inhibiting migration of a carcinoma cell in a subject. This method involves administering to the subject an inhibitor capable of blocking binding of a growth factor to at least one receptor for the growth factor under conditions effective to inhibit migration of the carcinoma cell.

The present invention also relates to a protein or polypeptide encoded by a nucleic acid molecule conferring on a mammalian carcinoma cell an ability to undergo cell migration.

The present invention also relates to an isolated antibody or binding portion thereof raised against a protein or polypeptide encoded by a nucleic acid molecule conferring on a mammalian carcinoma cell an ability to undergo cell migration.

The present invention also relates to a method for detecting the presence of a migrating carcinoma cell in a sample of a subject's tissue or body fluids. This method involves (1) providing a protein or polypeptide as an antigen, where the protein or polypeptide is encoded by a nucleic acid molecule conferring on a mammalian carcinoma cell an ability to undergo cell migration; (2) contacting the sample with the antigen; and (3) detecting any reaction which indicates that the migrating carcinoma cell is present in the sample using an assay system.

The present invention also relates to a method for detecting the presence of a migrating carcinoma cell in a sample of a subject's tissue or body fluids. This method involves (1) providing an antibody or binding portion thereof raised against a protein or polypeptide encoded by a nucleic acid molecule conferring on a mammalian carcinoma cell an ability to undergo cell migration; (2) contacting the sample with the antibody or binding portion thereof; and (3) detecting any reaction which indicates that the migrating carcinoma cell is present in the sample using an assay system.

The present invention also relates to a method for detecting the presence of a migrating carcinoma cell in a sample of a subject's tissue or body fluids. This method involves (1) providing a nucleotide sequence as a probe in a nucleic acid hybridization assay, where the nucleotide sequence is a nucleic acid molecule conferring on a mammalian carcinoma cell an ability to undergo cell migration; (2) contacting the sample with the probe; and (3) detecting any reaction which indicates that the migrating carcinoma cell is present in the sample.

The present invention also relates to a fourth method for detecting the presence of a migrating carcinoma cell in a sample of a subject's tissue or body fluids. This method involves (1) providing a nucleotide sequence as a probe in a gene amplification detection procedure, where the nucleotide sequence is a nucleic acid molecule conferring on a mammalian carcinoma cell an ability to undergo cell migration; (2) contacting the sample with the probe; and (3) detecting any reaction which indicates that the migrating carcinoma cell is present in the sample.

The present invention further relates to a first method of inhibiting the migration of placental cells into a blood stream of a mammalian subject. This method involves administering to the mammalian subject an antisense oligonucleotide complementary to a target portion of a nucleic acid molecule conferring on the placental cells an ability, in vivo, to undergo cell migration under conditions effective to inhibit migration of said placental cells into the blood stream. Suitable placental cells include, but are not limited to, cytotrophoblast cells.

The present invention also relates to a second method of inhibiting the migration of placental cells into a blood stream of a mammalian subject. This method involves administering to the mammalian subject an inhibitor capable of blocking binding of a growth factor to at least one receptor for the growth factor under conditions effective to inhibit migration of said placental cells. Suitable placental cells include, but are not limited to, cytotrophoblast cells.

The present invention also relates to a method of inducing the establishment of anchoring villi and blood supply to a mammalian fetus. This method involves transducing the ectopic expression of the nucleic acid molecule of the present invention using a suitable expression vector into cytotrophoblast cells or precursors thereof, under conditions effective to induce the establishment of anchoring villi and blood supply to a mammalian fetus.

The present invention further relates to a method of transgenically expressing the nucleic acid molecule of the present invention in a mammalian cell. This method involves cloning the nucleic acid molecule of the present invention into a suitable expression vector and transfecting the vector into a mammalian cell using suitable means of transfection, under conditions effective to transgenically express the nucleic acid molecule in a mammalian cell. Suitable means of transfection include, but are not limited to, electroporation, lipophilic reagent, and calcium chloride.

The present invention also relates to a method for detecting the presence of fetal cytotrophoblast cells in a sample of a subject's tissue or body fluids. This method involves providing a nucleotide sequence corresponding to the nucleic acid molecule of the present invention as a probe in a detection assay, contacting the sample with the probe, and detecting any reaction which indicates that fetal cytotrophoblast cells are present in the sample.

The discovery of carcinoma cell-specific targets that can be used to inhibit metastasis is important for developing methods of detecting and treating cancer. In furtherance of this pursuit, it would be helpful to identify HGF/Met-regulated genes that contribute to migration of carcinoma cells and to determine their signaling pathways. The Mig-7 cDNA is potentially the first carcinoma cell-specific cDNA that has been thus far identified. Evidence shows that Mig-7 plays a key role in migration of carcinoma cells. Furthermore, the degree of Mig-7 induction by HGF is determined by the differentiated state of integrin expression on the carcinoma cell in that αvβ5 binding also regulates Mig-7 expression. Studies have shown that (1) Mig-7 expression is only detected in carcinoma cells which tend to be less differentiated and (2) blocking antibody to αvβ5 also blocks HGF induction of Mig-7. This is significant, in that blockade of Mig-7 induction could lead to the development of new and innovative approaches to inhibit metastasis.

### BRIEF DESCRIPTION OF THE DRAWINGS

FIGS. 1A-1D show the nucleic acid sequence for the Mig-7 gene, the proposed amino acid sequence of the Mig-7 protein, and a hydrophobicity plot of the Mig-7 protein. FIG. 1A shows the Mig-7 cDNA sequence isolated by SSH and RACE. Region outlined by box is the proposed Kozak consensus sequence, a bold overline designates the stop codon, and dotted line box outlines the polyadenylation signal sequence. Following the last nucleotide is a string of 29 adenosines indicative of the Poly A tail (not shown). FIG. 11B shows homology comparisons between two existing expressed sequence tags (“ESTs”), N41315 to Mig-7 5′ and AI18969 to Mig-7 3′. FIG. 1C shows the proposed amino acid sequence of Mig-7. FIG. 1D shows a Kyte-Doolittle (Kyte et al., “A Simple Method for Displaying the Hydropathic Character of a Protein,” J. Mol. Bio. 157:105-132 (1982), which is incorporated by reference in its entirety) hydophobicity plot of Mig-7 protein, which predicts that amino acids 35-60 are within a transmembrane region.

FIGS. 2A-2G illustrate HGF-induced migration of RL95 cells and Mig-7 temporal expression induced by HGF in two different endometrial epithelial carcinoma cell lines, RL95 and HEC-1A. FIGS. 2A-2C show that RL95 cells start migrating at 12 hours of HGF treatment. RL95 cells grow in colonies of cells with some cells growing on top of others (star) and some cells in a monolayer (arrowheads). A representative colony was chosen to show migration of cells in that specific colony after treatment with HGF. Six fields of view per plate (n=3) were confirmed to show HGF-induced migration. Cells were photographed with a N70 Nikon camera with a Nikon inverted microscope at 20× objected and fitted with a GIF filter. FIG. 2A is a picture showing the morphology of the colony between 0 and 6 hours of treatment. FIG. 2B is at 12 hours of treatment. FIG. 2C panel is at 24 hours of treatment. Cells are noticeably rounded up by 12 hours (FIG. 2B) and single cells as well as cohorts of cells are migrating away from the colony. FIG. 2D is a representative Northern blot analysis of clone 34 and Mig-7 expression in total RNA isolated from RL95 cells treated with HGF for the indicated hours after serum starvation. Note induction of Mig-7 expression occurs prior to migration of both RL95. Each lane was loaded with 20 μg of total RNA. The blots were washed and exposed to film with two screens for 5 days at −70° C. FIG. 2E is a representative Northern blot analysis (n=3) of total RNA isolated from HGF treated HEC-LA cells for the indicated hours after serum starvation. FIGS. 2F and 2G are densitometry analyses of Northern blot analyses shown in FIG. 2B (Mig-7 only) and FIG. 2C, respectively.

FIGS. 3A-3B demonstrate Mig-7 expression in vivo. In FIG. 3A, metastatic tumors were homogenized in RNA STAT (TelTest) and RNA was isolated. RT-PCR was performed as described in the Examples infra. Products were run on an ethidium bromide stained, 1.5% agarose gel. Note that all tumors expressed c-Met and Mig-7. FIG. 3B shows RT-PCR results of metastatic tumor samples from additional patients.

FIGS. 4A-4B demonstrate that Mig-7 is not expressed in normal tissues and cannot be induced in primary endometrial epithelial cells. FIG. 4A shows a representative Mig-7 primer pair RT-PCR of various human tissues pooled from several individuals for each tissue. cDNA was in a 96-well format obtained from OriGene (Rockville, Md.). This experiment was performed twice with different 96-well plates. PCR with 18s primers of yet another 96-well plate confirms the presence of cDNA in each sample. Only the highest concentration of cDNA samples is shown. Lanes 1 and 14 are Low Mass Ladder (Gibco), lane 27 is cDNA from HGF-treated RL95 cells (24 hours). The same Mig-7 primers and PCR cycling parameters were used as described in the primary endometrial epithelial experiment. PCR products were run on an ethidium bromide 1.5% agarose gel. Tissues represented in the following lanes: 2, brain; 3, heart; 4, kidney, 5, spleen; 6, liver; 7, colon; 8, lung; 9, small intestine; 10, muscle; 11, stomach; 12, testis; 13, term placenta; 15, salivary gland; 16, thyroid; 17, adrenal; 18, pancreas; 19, ovary; 20, uterus; 21, prostate; 22, skin; 23, peripheral blood leukocytes; 24, bone marrow; 25, fetal brain; 26, fetal liver, 27, positive control HGF treated (12 hr) RL95 cells. PCR of all samples was simultaneous using a 96-well format and the same PCR reagent master mix. FIG. 4B shows an RT-PCR of pooled, primary endometrial epithelial cells as compared to RL95 cells treated with HGF. After documenting the gel, Mig-7 specificity of the amplified bands were confirmed by transferring the amplified cDNA to a membrane and Southern blotting with ³²P-labeled random primed Mig-7 cDNA probe. Note the lack of Mig-7 induction by HGF in the primary cells.

FIGS. 5A-5D demonstrate the effect of integrin blocking antibodies on Mig-7 expession. FIGS. 5A-5B, respectively, are representative Northern blot analysis and densitometry results of integrin blocking antibodies (Chemicon) and Mig-7 (˜1.6 kb) expression in RL95 cells after six hours of HGF treatment. The Northern blot was also stained with methylene blue to confirm that equivalent levels of RNA were loaded for each sample; this was the same result as shown with the actin probe (2.0 kb). Lane 1, no treatment; lane 2, 40 ng/mL HGF; lane 3, β1 antibody (GS6), no HGF; lane 4, 11 antibody+40 ng/mL HGF; lane 5, αvβ5 (P1F6) antibody, no HGF; lane 6, αvβ5 antibody+40 ng/mL HGF; lane 7, αvβ6 antibody (10D5), no HGF; lane 8, αvβ6 antibody+40 ng/mL HGF. All antibodies were used at a concentration of 8 μg/mL. FIGS. 5C-5D, respectively, are representative Northern blot analysis of clone 34 expression and densitometry results in RL95 cells treated as described above. Note that blocking antibodies to αvβ5 also blocked expression of clone 34 (lane 5 compared to lane 6). Probed blots were exposed to film for 4 days (Mig-7 and clone 34) or 1 day (actin) at −70° C. with two intensifying screens.

FIGS. 6A-6E show the comparison of HGF-induced migration of RL95 cells treated with Mig-7 specific antisense, irrelevant, or no oligonucleotides. FIG. 6A shows treatments: 1, no oligo; 2 and 3 are the two different Mig-7 specific antisense oligos, and 4 is the irrelevant oligo. Cell migration was decreased by Mig-7 antisense ODN treatment in a standard scratch migration assay. The migration of cells was quantitatively assessed 24 hours after the introduction of the scratch wound. The Y-axis represents the average number of cells invading the wound area per field of view (n=3) at 20× objective per well (n=3) for each treatment FIGS. 6B-6E document representative fields of view of RL95 cells treated as described in FIG. 6A by photomicroscopy as described previously in FIG. 2. The dotted lines represent the position of the initial scratch/wound.

FIG. 7 shows the detection of Mig-7 in the blood of a nude mouse injected with SF-treated RL95 cells. Mig-7 amplified cDNA was detected in one of three mice injected with SF treated RL95 cells in Matrigel. Only 1 μl of each RT reaction was used.

FIGS. 8A-8B show representative Northern analyses of RNA from RL-95 and HEC-1A cells, respectively, treated with SF as described previously. Each lane was loaded with 20 μg of total RNA from cells treated for the indicated time. The blots were probed with random primed ³²P-labeled Mig-7 cDNA, washed and exposed to film with two screens for 5 days at −70° C.

FIG. 9 shows a representative Mig-7 primer pair RT-PCR of various human tissues pooled from several individuals for each tissue. cDNA was in a 96-well format obtained from OriGene (Rockville, Md.). This experiment was performed twice with different 96-well plates. Lanes 1 and 14 are Low Mass Ladder (Gibco), lane 27 is cDNA from SF-treated RL-95 cells (24 hours). The same Mig-7 primers and PCR cycling parameters were used as described in the primary endometrial epithelial experiment. PCR products were run on an ethidium bromide 1.5% agarose gel. Lane 2, brain; lane 3, heart; lane 4, kidney; lane 5, spleen; lane 6, liver; lane 7, colon; lane 8, lung; lane 9, small intestine; lane 10, muscle; lane 11, stomach; lane 12, testis; lane 13, placenta; lane 15, salivary gland; lane 16, thyroid; lane 17, adrenal; lane 18, pancreas; lane 19, ovary; lane 20, uterus; lane 21, prostate; lane 22, skin; lane 23, peripheral blood leukocytes; lane 24, bone marrow; lane 25, fetal brain; lane 26, fetal liver; lane 27, positive control (upper tier only) SF treated RL-95 cells. PCR of all samples was simultaneous using a 96-well format and the same PCR reagent master mix.

FIGS. 10A-10C demonstrate that detectable Mig-7 mRNA expression corresponds to Met mRNA expression. In FIG. 10A, tissues were homogenized in RNA STAT (TelTest) and RNA was isolated. RT-PCR was performed with the same Mig-7 and Met primers as previously described. PCR products were run on an ethidium bromide stained, 1.5% agarose gel. FIG. 10B shows a cancer profiling array hybridized at 65° C. overnight with random primed, ³²P-labeled Mig-7 cDNA probe and washed under stringent conditions (at 65° C. for two hours with four changes of 2×SSC, 0.5% SDS solution and at 65° C. for 30 minutes with 0.2×SSC, 0.5% SDS solution. After washing, the array was exposed to film for 10 days with two screens at −70° C. then the film was developed and scanned. The numbered columns contain samples from the following cancer types: 1-breast, 2-uterus, 3-colon, 4-stomach, 5-ovary with one cervical at bottom, 6-lung, 7-kidney, 8-rectum/small intestine, and 9-thyroid/prostate/pancreas. S=tissue surrounding tumor, T=tumor, G-genomic DNA (positive control). FIG. 10C shows detection of Mig-7 expression in human blood RNA samples from cancer patients (lanes 1-5) and normal individuals (lanes 6-8). The upper tier of the upper panel are RT-PCR of the RNA from indicated individuals. The lower tier of the upper panel is the PCR of samples that were not reverse transcribed (a control for contaminating genomic DNA). The lower panel is RT-PCR using primers for 18s ribosomal RNA (Ambion, 34 cycles) and the same RT reaction for each sample used in the upper tier of the upper panel, which shows intact reverse transcribed RNA (cDNA) for each sample. Dark regions on upper panel are due to dye fronts on gel which do not interfere with UV detection of ethidium bromide stained DNA.

FIG. 11 shows Densitometry results of integrin blocking antibodies (all obtained from Chemicon) with respect to Mig-7 expression in RL-95 cells with six hours of SF treatment normalized to actin. The Northern blot was also stained with methylene blue to confirm equal levels of RNA loaded for each sample. This was the same result obtained with the actin probe. Lane 1, no treatment; lane 2, 40 ng/mL SF; lane 3, β1 antibody (GS6) no SF; land 4, β1 antibody+40 ng/mL SF; lane 5, β1 antibody+80 ng/mL SF; lane 6, αvβ5 antibody (P1F6); lane 7, αvβ5 antibody+40 ng/mL SF, lane 8, αvβ5 antibody+80 ng/mL SF; lane 9, αvβ6 antibody (10D5); lane 10, αvβ6 antibody+40 ng/mL SF; lane 11, αvβ6 antibody+80 ng/mL SF; lane 12, 6 hour treatment from prior SF experiment for a positive control of SF activity. All antibodies were used at a concentration of 8 μg/mL media as recommended by Chemicon.

FIG. 12 is a graph showing the results of a cell migration inhibition study using antisense oligonucleotides of Mig-7. RL-95 cells were plated in six well plates at 70% confluency. After attachment, cells were treated with serum-free, phenol-free DMEM for 48 hours. Each oligo in FuGene (as directed by Roche) was used at 1 μg per well in one mL of media. After 15 minutes, 50 ng/mL of Scatter Factor (“SF”) was added. A wounded area was created in each well with a pipette tip. Migrated cells were counted 24 hours later. Treatments: 1 is no oligo, 2 and 3 are two different Mig-7 specific antisense oligos, and 4 is the irrelevant oligo.

FIG. 13 is a graph showing the results of a study of the effect of antisense Mig-7 treatment on tumor size. Nude mice (Jackson Labs) were injected with (1) Mig-7 specific antisense oligonucleotide, (2) control oligonucleotide, or (3) no oligonucleotide treated RL-95 cells stimulated with SF in Matrigel. Another control was five animals injected with Matrigel alone that contained 50 ng/mL SF; one of these control animals had a tumor.

FIG. 14 is a diagram showing that insulin like growth factor (“ILGF”) and epidermal growth factor (“EGF”) upregulate Mig-7 in αvβ5 positive pancreatic carcinoma cells. Lanes: HGF, represents the hepatocyte growth factor positive control treatment; EGF, represents the EGF treatment; and ILGF, represents the ILGF treatment. The first lane of each treatment corresponds to 6 hours of treatment. The second lane of each treatment corresponds to 24 hours of treatment. The third lane of each treatment corresponds to 50 hours of treatment.

FIG. 15 shows Mig-7 expression in early placenta at 7 weeks of gestation. Invasion of cytotrophoblast cells ceases by 22 weeks of gestation (Zhou et al., “Human Cytotrophoblasts Adopt a Vascular Phenotype As They Differentiate,” Journal of Clinical Investigation 99(9):2139-2151 (1997), which is hereby incorporated by reference in its entirety). Thus, the lack of Mig-7 expression at 38 weeks is consistent with Mig-7 induction by HGF, cytotrophoblast cells expressing αvβ5 integrin (Zhou et al., “Human Cytotrophoblasts Adopt a Vascular Phenotype As They Differentiate,” Journal of Clinical Investigation 99(9):2139-2151 (1997), which is hereby incorporated by reference in its entirety) and cytotropholblast cells ceasing migration by this week of gestation (Dolras et al., “Regulation of Human Cytotrophoblast Morphogenesis Hepatocyte Growth Factor/Scatter Factor,” Biology of Reproduction 65:1278-1288 (2001), which is hereby incorporated by reference in its entirety). RT-PCR was performed as previously described for FIGS. 3A, 3B, and 4B).

### DETAILED DESCRIPTION OF THE INVENTION

The present invention relates to an isolated nucleic acid molecule conferring on a mammalian carcinoma cell an ability to undergo cell migration. One aspect of the present invention relates to the isolation and identification of a nucleic acid molecule encoding a mammalian migration inducting gene (generally referred to herein as “Mig-7”). Mig-7 is upregulated by Hepatocyte Growth Factor (“HGF”), also known as Scatter Factor (“SF”). The Mig-7 gene protein or polypeptide products (generally referred to as Mig-7) are involved in cancer cell migration. The Mig-7 gene has numerous open reading frames (“ORFs”), as described infra (see Kozak, “The Scanning Model for Translation: An Update,” Journal of Cell Biology 108:229-241 (1989), which is hereby incorporated by reference in its entirety).

In one embodiment, the nucleic acid molecule of the present invention comprises a nucleotide sequence corresponding to SEQ ID NO-1 as follows:

| | | | | | | |
|---|---|---|---|---|---|---|
| gaaaagtcct | tggctttgaa | agacgaatga | tgagcagttc | agtggcccat | gtcacagtcc | 60 |
| aggcacctgc | caaaggtgac | tccctgggag | gagcatctta | gtcacagagc | cagtgcctgc | 120 |
| tgtaggtgtg | cagaagggtg | catgtgtgtg | tgtgtgtgtg | tgtgtgtatg | tgtacgtgta | 180 |
| catgtgtgtt | gggggaaggg | agcaagggtt | gtgggagcat | ttcttatctg | ctcttctctg | 240 |
| caagatttcc | tgtgatttaa | gtcacattaa | agtacccata | agcccgtaat | gcaaaagaac | 300 |
| cccaaaacca | gcccagcagc | caaccatggc | agcaagtaga | tgctctggtc | tttacatagt | 360 |
| cagaaatgac | acttctgggc | tctcaggcag | tcagtgggtt | gactccccat | taaagccccc | 420 |
| tgccaagtct | ggaatagtcc | tagtcccgtg | tgtgtgtgtg | tgtgtgtgtg | tgtgtgtgtg | 480 |
| tgtgtgtgtg | tgtgtgtacc | cgcgtgcata | tgcgcgcatg | cagtgcaggg | tctgcatacc | 540 |
| taaagcagat | gaaattctgc | agaatggctg | cctcactaga | caaagtcaag | aagacagacc | 600 |
| gaggagagag | aggttgatgt | gtctccacta | ccaagagata | ggcttctcta | agccagcgag | 660 |
| acatcccatc | caacaatatg | aaactggcca | catttccttg | agatgtcaac | gtgaaagtgt | 720 |
| agctgcatct | ttattcttca | ctgttatgaa | gttgggtgca | acacagcttg | agtggaatac | 780 |
| aaaacaccgc | ttggaaacac | atgatctgga | tttgaatcgc | agctgtatca | ttcacctgct | 840 |
| atgagacttt | gagcaagacc | tctctgaggt | tatttcttca | cagtaggtag | agacaagact | 900 |
| tacttcaaag | gttcttaaag | ttgaacctga | gtcaatgaat | gcaaaagtgt | tcacatttaa | 960 |
| actgtaattt | taaagcacaa | tacaagtaaa | tagcattaat | atcattagag | agattaactt | 1020 |
| agcactgtgc | gtcacatgat | tcatcacggg | ccatctgtga | gatatcaaat | agagaggtga | 1080 |
| agcctgcagt | aataaaaaat | actgccatag | ctata | | | 1115 |

In another embodiment, the nucleic acid molecule of the present invention comprises a nucleotide sequence corresponding to SEQ ID NO:2 as follows:

| | | | | | | |
|---|---|---|---|---|---|---|
| gtgcctctct | atggagagca | cctctgtggc | ctctctgaga | gcactcacag | ccaaaagtac | 60 |
| acagctgccc | ccaggctgag | agtgcttgat | acacccttga | atcccctctt | atatgatgcc | 120 |
| ccagcccagg | agagataaaa | gcatcagcac | catgagattc | acctgcctct | ggtcgttagg | 180 |
| gaacaatgga | ggcctgcgat | tggagttaaa | ctctcagtga | tctctgtgtt | gacaacacca | 240 |
| aagctagagg | aatccagtag | gatgtgggca | tggttttccc | ggaaggctga | ctgagcagtt | 300 |
| ctgcaaatgt | ttgcaagtac | agggcagaat | ttcatccagc | ctcagaacct | tgagccaaga | 360 |
| ctcagcatca | gcaaagccaa | aagtttcatt | tcttcgactg | tgggagtgct | agtcccaacc | 420 |
| tttagatggc | cattcagttt | taagttcaat | aagcattttg | attgagaaat | actttgctga | 480 |
| ggagtgaaaa | gtccttggct | ttgaaagacg | aatgatgagc | agttcagtgg | cccatgtcac | 540 |
| agtccaggca | cctgccaaag | gtgactccct | gggaggagca | tcttagtcac | agagccagtg | 600 |
| cctgctgtag | gtgtgcagaa | gggtgcatgt | gtgtgtgtgt | gtgtgtgtgt | gtatgtgtac | 660 |
| gtgtacatgt | gtgttggggg | aagggagcaa | gggttgtggg | agcatttctt | atctgctctt | 720 |
| ctctgcaaga | tttcctgtga | tttaagtcac | attaaagtac | ccataagccc | gtaatgcaaa | 780 |
| agaaccccaa | aaccagccca | gcagccaacc | atggcagcaa | gtagatgctc | tggtctttac | 840 |
| atagtcagaa | atgacacttc | tgggctctca | ggcagtcagt | gggttgactc | cccattaaag | 900 |
| ccccctgcca | agtctggaat | agtcctagtc | ccgtgtgtgt | gtgtgtgtgt | gtgtgtgtgt | 960 |
| gtgtgtgtgt | gtgtgtgtgt | gtacccgcgt | gcatatgcgc | gcatgcagtg | cagggtctgc | 1020 |
| atacctaaag | cagatgaaat | tctgcagaat | ggctgcctca | ctagacaaag | tcaagaagac | 1080 |
| agaccgagga | gagagaggtt | gatgtgtctc | cactaccaag | agataggctt | ctctaagcca | 1140 |
| gcgagacatc | ccatccaaca | atatgaaact | ggccacattt | ccttgagatg | tcaacgtgaa | 1200 |
| agtgtagctg | catctttatt | cttcactgtt | atgaagttgg | gtgcaacaca | gcttgagtgg | 1260 |
| aatacaaaac | accgcttgga | aacacatgat | ctggatttga | atcgcagctg | tatcattcac | 1320 |
| ctgctatgag | actttgagca | agacctctct | gaggttattt | cttcacagta | ggtagagaca | 1380 |
| agacttactt | caaaggttct | taaagttgaa | cctgagtcaa | tgaatgcaaa | agtgttcaca | 1440 |
| tttaaactgt | aattttaaag | cacaatacaa | gtaaatagca | ttaatatcat | tagagagatt | 1500 |
| aacttagcac | tgtgcgtcac | atgattcatc | acgggccatc | tgtgagatat | caaatagaga | 1560 |
| ggtgaagcct | gcagtaataa | aaaatactgc | catagctata | | | 1600 |

Mig-7 is an SF/c-Met regulated, cancer cell-specific expressed gene. This gene is not detectably expressed in normal tissues. Because this gene is expressed prior to migration, it is a target for inhibiting migration of cancer cells while allowing maintenance of normal cell functions. Cancer cell migration in culture can be inhibited by using molecules that inhibit expression and, therefore, function of the Mig-7 gene. In addition, the Mig-7 gene can be used to detect migrating cancer cells in the blood of metastatic cancer patients, thereby providing a non-invasive method for detection of metastases. Finally, using Mig-7 as a marker, migrating cancer cells can be detected in pathologist-evaluated, “normal” tissue adjacent to the tumor. Such an assay can provide a molecular means of determining if all of the tumor cells have been surgically removed. This method can provide a means to target and inhibit the spread of cancer cells.

Thus, important advances in the therapy of metastatic cancer are a reasonable expectation in view of the isolation and characterization of Mig-7. Such advances will be of significance because what is learned will contribute to a broader understanding of how integrins interact with SF and c-Met to cause cell specific responses.

The present invention also relates to the proteins or polypeptides encoded by Mig-7. In one embodiment, the Mig-7 gene having the nucleotide sequence of SEQ ID NO:2 has at least 28 open reading frames (“ORFs”) encoding at least 28 different Mig-7 proteins or polypeptides, as described below.

In one embodiment, the protein or polypeptide of the present invention has an amino acid sequence corresponding to SEQ ID NO:3 as follows:

```
Met Ala Ala Ser Arg Cys Ser Gly Leu Tyr Ile Val Arg Asn Asp Thr
    1 5 10 15
Ser Gly Leu Ser Gly Ser Gln Trp Val Asp Ser Pro Leu Lys Pro Pro
                          20 25 30
Ala Lys Ser Gly Ile Val Leu Val Pro Cys Val Cys Val Cys Val Cys
                  35 40 45
Val Cys Val Cys Val Cys Val Cys Val Tyr Pro Arg Ala Tyr Ala Arg
          50 55 60
Met Gln Cys Arg Val Cys Ile Pro Lys Ala Asp Glu Ile Leu Gln Asn
  65 70 75 80
Gly Cys Leu Thr Arg Gln Ser Gln Glu Asp Arg Pro Arg Arg Glu Arg
                                  85 90 95
Leu Met Cys Leu His Tyr Gln Glu Ile Gly Phe Ser Lys Pro Ala Arg
                        100 105 110
His Pro Ile Gln Gln Tyr Glu Thr Gly His Ile Ser Leu Arg Cys Gln
                115 120 125
Arg Glu Ser Val Ala Ala Ser Leu Phe Phe Thr Val Met Lys Leu Gly
        130 135 140
Ala Thr Gln Leu Glu Trp Asn Thr Lys His Arg Leu Glu Thr His Asp
145 150 155 160
Leu Asp Leu Asn Arg Ser Cys Ile Ile His Leu Leu
                                165 170
```

This protein or polypeptide has an estimated molecular weight of approximately 20 to 40 kilodaltons, and preferably about 21.66 kilodaltons, based on the deduced amino acid sequence, and is encoded by the ORF corresponding to nucleotide bases 808 to 1327 of SEQ ID NO:2.

In another embodiment, the protein or polypeptide of the present invention has an amino acid sequence corresponding to SEQ ID NO:4 as follows:

```
Val Pro Leu Tyr Gly Glu His Leu Cys Gly Leu Ser Glu Ser Thr His
    1 5 10 15
Ser Gln Lys Tyr Thr Ala Ala Pro Arg Leu Arg Val Leu Asp Thr Pro
                          20 25 30
Leu Asn Pro Leu Leu Tyr Asp Ala Pro Ala Gln Glu Arg
                  35 40 45
```

This protein or polypeptide has an estimated molecular weight of approximately 4 to 10 kilodaltons, and preferably about 5.13 kilodaltons, based on the deduced amino acid sequence, and is encoded by the ORF corresponding to nucleotide bases 1 to 136 of SEQ ID NO:2.

In another embodiment, the protein or polypeptide of the present invention has an amino acid sequence corresponding to SEQ ID NO:5 as follows:

```
Lys His Gln His His Glu Ile His Leu Pro Leu Val Val Arg Glu Gln
    1 5 10 15
Trp Arg Pro Ala Ile Gly Val Lys Leu Ser Val Ile Ser Val Leu Thr
                          20 25 30
Thr Pro Lys Leu Glu Glu Ser Ser Arg Met Trp Ala Trp Phe Ser Arg
                  35 40 45
Lys Ala Asp
          50
```

This protein or polypeptide has an estimated molecular weight of approximately 4 to 10 kilodaltons, and preferably about 5.81 kilodaltons, based on the deduced amino acid sequence, and is encoded by the ORF corresponding to nucleotide bases 136 to 292 of SEQ ID NO:2.

In another embodiment, the protein or polypeptide of the present invention has an amino acid sequence corresponding to SEQ ID NO:6 as follows:

```
Ala Val Leu Gln Met Phe Ala Ser Thr Gly Gln Asn Phe Ile Gln Pro
    1 5 10 15
Gln Asn Leu Glu Pro Arg Leu Ser Ile Ser Lys Ala Lys Ser Phe Ile
                          20 25 30
Ser Ser Thr Val Gly Val Leu Val Pro Thr Phe Arg Trp Pro Phe Ser
                  35 40 45
Phe Lys Phe Asn Lys His Phe Asp
          50 5
```

or polypeptide has an estimated molecular weight of approximately 5 to 11 kilodaltons, and preferably about 6.38 kilodaltons, based on the deduced amino acid sequence, and is encoded by the ORF corresponding to nucleotide bases 292 to 463 of SEQ ID NO:2.

In another embodiment, the protein or polypeptide of the present inventions has an amino acid sequence corresponding to SEQ ID NO:7 as follows:

```
Leu Pro Gly Arg Ser Ile Leu Val Thr Glu Pro Val Pro Ala Val Gly
    1 5 10 15
Val Gln Lys Gly Ala Cys Val Cys Val Cys Val Cys Val Tyr Val Tyr
                          20 25 30
Val Tyr Met Cys Val Gly Gly Arg Glu Gln Gly Leu Trp Glu His Phe
                  35 40 45
Leu Ser Ala Leu Leu Cys Lys Ile Ser Cys Asp Leu Ser His Ile Lys
          50 55 60
Val Pro Ile Ser Pro
  65
```

This protein or polypeptide has an estimated molecular weight of approximately 6 to 12 kilodaltons, and preferably about 7.87 kilodaltons, based on the deduced amino acid sequence, and is encoded by the ORF corresponding to nucleotide bases 562 to 772 of SEQ ID NO:2.

In another embodiment, the protein or polypeptide of the present invention has an amino acid sequence corresponding to SEQ ID NO:8 as follows:

```
Cys Lys Arg Thr Pro Lys Pro Ala Gln Gln Pro Thr Met Ala Ala Ser
    1 5 10 15
Arg Cys Ser Gly Leu Tyr Ile Val Arg Asn Asp Thr Ser Gly Leu Ser
                          20 25 30
Gly Ser Gln Trp Val Asp Ser Pro Leu Lys Pro Pro Ala Lys Ser Gly
                  35 40 45
Ile Val Leu Val Pro Cys Val Cys Val Cys Val Cys Val Cys Val Cys
          50 55 60
Val Cys Val Cys Val Tyr Pro Arg Ala Tyr Ala Arg Met Gln Cys Arg
  65 70 75 80
Val Cys Ile Pro Lys Ala Asp Glu Ile Leu Gln Asn Gly Cys Leu Thr
                                  85 90 95
Arg Gln Ser Gln Glu Asp Arg Pro Arg Arg Glu Arg Leu Met Cys Leu
                        100 105 110
His Tyr Gln Glu Ile Gly Phe Ser Lys Pro Ala Arg His Pro Ile Gln
                115 120 125
Gln Tyr Glu Thr Gly His Ile Ser Leu Arg Cys Gln Arg Glu Ser Val
        130 135 140
Ala Ala Ser Leu Phe Phe Thr Val Met Lys Leu Gly Ala Thr Gln Leu
145 150 155 160
Glu Trp Asn Thr Lys His Arg Leu Glu Thr His Asp Leu Asp Leu Asn
                                165 170 175
Arg Ser Cys Ile Ile His Leu Leu
                        180
```

This protein or polypeptide has an estimated molecular weight of approximately 19 to 40 kilodaltons, and preferably about 20.98 kilodaltons, based on the deduced amino acid sequence, and is encoded by the ORF corresponding to nucleotide bases 772 to 1327 of SEQ ID NO:2.

In another embodiment, the protein or polypeptide of the present invention has an amino acid sequence corresponding to SEQ ID NO:9 as follows:

```
Ile Pro Ser Tyr Met Met Pro Gln Pro Arg Arg Asp Lys Ser Ile Ser
    1 5 10 15
Thr Met Arg Phe Thr Cys Leu Trp Ser Leu Gly Asn Asn Gly Gly Leu
                          20 25 30
Arg Leu Glu Leu Asn Ser Gln
                  35
```

This protein or polypeptide has an estimated molecular weight of approximately 3 to 9 kilodaltons, and preferably about 4.45 kilodaltons, based on the deduced amino acid sequence, and is encoded by the ORF corresponding to nucleotide bases 98 to 218 of SEQ ID NO:2.

In another embodiment, the protein or polypeptide of the present invention has an amino acid sequence corresponding to SEQ ID NO:10 as follows:

```
Arg Asn Pro Val Gly Cys Gly His Gly Phe Pro Gly Arg Leu Thr Glu
    1 5 10 15
Gln Phe Cys Lys Cys Leu Gln Val Gln Gly Arg Ile Ser Ser Ser Leu
                          20 25 30
Arg Thr Leu Ser Gln Asp Ser Ala Ser Ala Lys Pro Lys Val Ser Phe
                  35 40 45
Leu Arg Leu Trp Glu Cys
          50
```

This protein or polypeptide has an estimated molecular weight of approximately 5 to 11 kilodaltons, and preferably about 6.16 kilodaltons, based on the deduced amino acid sequence, and is encoded by the ORF corresponding to nucleotide bases 245 to 410 of SEQ ID NO:2.

In another embodiment, the protein or polypeptide of the present invention has an amino acid sequence corresponding to SEQ ID NO:11 as follows:

```
Val Cys Arg Arg Val His Val Cys Val Cys Val Cys Val Cys Met Cys
    1 5 10 15
Thr Cys Thr Cys Val Leu Gly Glu Gly Ser Lys Gly Cys Gly Ser Ile
                          20 25 30
Ser Tyr Leu Leu Phe Ser Ala Arg Phe Pro Val Ile
                  35 40
```

This protein or polypeptide has an estimated molecular weight of approximately 4 to 10 kilodaltons, and preferably about 5.02 kilodaltons, based on the deduced amino acid sequence, and is encoded by the ORF corresponding to nucleotide bases 608 to 743 of SEQ ID NO:2.

In another embodiment, the protein or polypeptide of the present invention has an amino acid sequence corresponding to SEQ ID NO:12 as follows:

```
Ser Arg Val Cys Val Cys Val Cys Val Cys Val Cys Val Cys Val Cys
    1 5 10 15
Val Cys Thr Arg Val His Met Arg Ala Cys Ser Ala Gly Ser Ala Tyr
                          20 25 30
Leu Lys Gln Met Lys Phe Cys Arg Met Ala Ala Ser Leu Asp Lys Val
                  35 40 45
Lys Lys Thr Asp Arg Gly Glu Arg Gly
          50 55
```

This protein or polypeptide has an estimated molecular weight of approximately 5 to 11 kilodaltons, and preferably about 6.50 kilodaltons, based on the deduced amino acid sequence, and is encoded by the ORF corresponding to nucleotide bases 926 to 1100 of SEQ ID NO:2.

In another embodiment, the protein or polypeptide of the present invention has an amino acid sequence corresponding to SEQ ID NO:13 as follows:

```
Ala Val Gln Trp Pro Met Ser Gln Ser Arg His Leu Pro Lys Val Thr
    1 5 10 15
Pro Trp Glu Glu His Leu Ser His Arg Ala Ser Ala Cys Cys Arg Cys
                          20 25 30
Ala Glu Gly Cys Met Cys Val Cys Val Cys Val Cys Val Cys Val Arg
                  35 40 45
Val His Val Cys Trp Gly Lys Gly Ala Arg Val Val Gly Ala Phe Leu
          50 55 60
Ile Cys Ser Ser Leu Gln Asp Phe Leu
  65 70
```

This protein or polypeptide has an estimated molecular weight of approximately 7 to 13 kilodaltons, and preferably about 8.32 kilodaltons, based on the deduced amino acid sequence, and is encoded by the ORF corresponding to nucleotide bases 516 to 738 of SEQ ID NO:2.

In another embodiment, the protein or polypeptide of the present invention has an amino acid sequence corresponding to SEQ ID NO:14 as follows:

```
Leu Pro Ile Lys Ala Pro Cys Gln Val Trp Asn Ser Pro Ser Pro Val
    1 5 10 15
Cys Val Cys Val Cys Val Cys Val Cys Val Cys Val Cys Val Cys Val
                          20 25 30
Pro Ala Cys Ile Cys Ala His Ala Val Gln Gly Leu His Thr
                  35 40 45
```

This protein or polypeptide has an estimated molecular weight of approximately 4 to 10 kilodaltons, and preferably about 5.24 kilodaltons, based on the deduced amino acid sequence, and is encoded by the ORF corresponding to nucleotide bases 885 to 1026 of SEQ ID NO:2.

In another embodiment, the protein or polypeptide of the present invention has an amino acid sequence corresponding to SEQ ID NO:15 as follows:

```
Val Cys Arg Pro Cys Thr Ala Cys Ala His Met His Ala Gly Thr His
    1 5 10 15
Thr His Thr His Thr His Thr His Thr His Thr His Thr His Thr Gly
                          20 25 30
Leu Gly Leu Phe Gln Thr Trp Gln Gly Ala Leu Met Gly Ser Gln Pro
                  35 40 45
Thr Asp Cys Leu Arg Ala Gln Lys Cys His Phe
          50 55
```

This protein or polypeptide has an estimated molecular weight of approximately 5 to 11 kilodaltons, and preferably about 6.73 kilodaltons, based on the deduced amino acid sequence, and is encoded by the ORF corresponding to nucleotide bases 1027 to 847 of SEQ ID NO:2.

In another embodiment, the protein or polypeptide of the present invention has an amino acid sequence corresponding to SEQ ID NO:16 as follows:

```
Cys Asp Leu Asn His Arg Lys Ser Cys Arg Glu Glu Gln Ile Arg Asn
    1 5 10 15
Ala Pro Thr Thr Leu Ala Pro Phe Pro Gln His Thr Cys Thr Arg Thr
                          20 25 30
His Thr His Thr His Thr His Thr His Met His Pro Ser Ala His Leu
                  35 40 45
Gln Gln Ala Leu Ala Leu
          50
```

This protein or polypeptide has an estimated molecular weight of approximately 5 to 11 kilodaltons, and preferably about 6.16 kilodaltons, based on the deduced amino acid sequence, and is encoded by the ORF corresponding to nucleotide bases 754 to 589 of SEQ ID NO:2.

In another embodiment, the protein or polypeptide of the present invention has an amino acid sequence corresponding to SEQ ID NO:17 as follows:

```
Asn Ser Ala Leu Tyr Leu Gln Thr Phe Ala Glu Leu Leu Ser Gln Pro
    1 5 10 15
Ser Gly Lys Thr Met Pro Thr Ser Tyr Trp Ile Pro Leu Ala Leu Val
                          20 25 30
Leu Ser Thr Gln Arg Ser Leu Arg Val
                  35 40
```

This protein or polypeptide has an estimated molecular weight of approximately 3 to 9 kilodaltons, and preferably about 4.67 kilodaltons, based on the deduced amino acid sequence, and is encoded by the ORF corresponding to nucleotide bases 334 to 208 of SEQ ID NO:2.

In another embodiment, the protein or polypeptide of the present invention has an amino acid sequence corresponding to SEQ ID NO:18 as follows:

```
Arg Pro Glu Ala Gly Glu Ser His Gly Ala Asp Ala Phe Ile Ser Pro
    1 5 10 15
Gly Leu Gly His His Ile Arg Gly Asp Ser Arg Val Tyr Gln Ala Leu
                          20 25 30
Ser Ala Trp Gly Gln Leu Cys Thr Phe Gly Cys Glu Cys Ser Gln Arg
                  35 40 45
Gly His Arg Gly Ala Leu His Arg Glu Ala
          50 55
```

This protein or polypeptide has an estimated molecular weight of approximately 5 to 11 kilodaltons, and preferably about 6.61 kilodaltons, based on the deduced amino acid sequence, and is encoded by the ORF corresponding to nucleotide bases 178 to 1 of SEQ ID NO:2.

In another embodiment, the protein or polypeptide of the present invention has an amino acid sequence corresponding to SEQ ID NO:19 as follows:

```
Arg Cys Ser Tyr Thr Phe Thr Leu Thr Ser Gln Gly Asn Val Ala Ser
    1 5 10 15
Phe Ile Leu Leu Asp Gly Met Ser Arg Trp Leu Arg Glu Ala Tyr Leu
                          20 25 30
Leu Val Val Glu Thr His Gln Pro Leu Ser Pro Arg Ser Val Phe Leu
                  35 40 45
Thr Leu Ser Ser Glu Ala Ala Ile Leu Gln Asn Phe Ile Cys Phe Arg
          50 55 60
Tyr Ala Asp Pro Ala Leu His Ala Arg Ile Cys Thr Arg Val His Thr
  65 70 75 80
His Thr His Thr His Thr His Thr His Thr His Thr His Thr Arg Asp
                                  85 90 95
```

This protein or polypeptide has an estimated molecular weight of approximately 9 to 19 kilodaltons, and preferably about 10.94 kilodaltons, based on the deduced amino acid sequence, and is encoded by the ORF corresponding to nucleotide bases 1218 to 927 of SEQ ID NO:2.

In another embodiment, the protein or polypeptide of the present invention has an amino acid sequence corresponding to SEQ ID NO:20 as follows:

```
Glu Pro Arg Ser Val Ile Ser Asp Tyr Val Lys Thr Arg Ala Ser Thr
    1 5 10 15
Cys Cys His Gly Trp Leu Leu Gly Trp Phe Trp Gly Ser Phe Ala Leu
                          20 25 30
Arg Ala Tyr Gly Tyr Phe Asn Val Thr
                  35 40
```

This protein or polypeptide has an estimated molecular weight of approximately 3 to 9 kilodaltons, and preferably about 4.67 kilodaltons, based on the deduced amino acid sequence, and is encoded by the ORF corresponding to nucleotide bases 870 to 744 of SEQ ID NO:2.

In another embodiment, the protein or polypeptide of the present invention has an amino acid sequence corresponding to SEQ ID NO:21 as follows:

```
Glu Met Leu Pro Gln Pro Leu Leu Pro Ser Pro Asn Thr His Val His
    1 5 10 15
Val His Ile His Thr His Thr His Thr His Thr Cys Thr Leu Leu His
                          20 25 30
Thr Tyr Ser Arg His Trp Leu Cys Asp
                  35 40
```

This protein or polypeptide has an estimated molecular weight of approximately 3 to 9 kilodaltons, and preferably about 4.67 kilodaltons, based on the deduced amino acid sequence, and is encoded by the ORF corresponding to nucleotide bases 711 to 585 of SEQ D NO:2.

In another embodiment, the protein or polypeptide of the present invention has an amino acid sequence corresponding to SEQ ID NO:22 as follows:

```
Asp Ala Pro Pro Arg Glu Ser Pro Leu Ala Gly Ala Trp Thr Val Thr
    1 5 10 15
Trp Ala Thr Glu Leu Leu Ile Ile Arg Leu Ser Lys Pro Arg Thr Phe
                          20 25 30
His Ser Ser Ala Lys Tyr Phe Ser Ile Lys Met Leu Ile Glu Leu Lys
                  35 40 45
Thr Glu Trp Pro Ser Lys Gly Trp Asp
          50 55
```

This protein or polypeptide has an estimated molecular weight of approximately 5 to 11 kilodaltons, and preferably about 6.50 kilodaltons, based on the deduced amino acid sequence, and is encoded by the ORF corresponding to nucleotide bases 585 to 411 of SEQ ID NO:2.

In another embodiment, the protein or polypeptide of the present invention has an amino acid sequence corresponding to SEQ ID NO:23 as follows:

```
His Ser His Ser Arg Arg Asn Glu Thr Phe Gly Phe Ala Asp Ala Glu
    1 5 10 15
Ser Trp Leu Lys Val Leu Arg Leu Asp Glu Ile Leu Pro Cys Thr Cys
                          20 25 30
Lys His Leu Gln Asn Cys Ser Val Ser Leu Pro Gly Lys Pro Cys Pro
                  35 40 45
His Pro Thr Gly Phe Leu
          50
```

This protein or polypeptide has an estimated molecular weight of approximately 5 to 11 kilodaltons, and preferably about 6.16 kilodaltons, based on the deduced amino acid sequence, and is encoded by the ORF corresponding to nucleotide bases 411 to 246 of SEQ ID NO:2.

In another embodiment, the protein or polypeptide of the present invention has an amino acid sequence corresponding to SEQ ID NO:24 as follows:

```
Leu Trp Gln Tyr Phe Leu Leu Leu Gln Ala Ser Pro Leu Tyr Leu Ile
    1 5 10 15
Ser His Arg Trp Pro Val Met Asn His Val Thr His Ser Ala Lys Leu
                          20 25 30
Ile Ser Leu Met Ile Leu Met Leu Phe Thr Cys Ile Val Leu
                  35 40 45
```

This protein or polypeptide has an estimated molecular weight of approximately 4 to 10 kilodaltons, and preferably about 5.24 kilodaltons, based on the deduced amino acid sequence, and is encoded by the ORF corresponding to nucleotide bases 1598 to 1457 of SEQ ID NO:2.

In another embodiment, the protein or polypeptide of the present invention has an amino acid sequence corresponding to SEQ ID NO:25 as follows:

```
Asn Tyr Ser Leu Asn Val Asn Thr Phe Ala Phe Ile Asp Ser Gly Ser
    1 5 10 15
Thr Leu Arg Thr Phe Glu Val Ser Leu Val Ser Thr Tyr Cys Glu Glu
                          20 25 30
Ile Thr Ser Glu Arg Ser Cys Ser Lys Ser His Ser Arg
                  35 40 45
```

This protein or polypeptide has an estimated molecular weight of approximately 4 to 10 kilodaltons, and preferably about 5.13 kilodaltons, based on the deduced amino acid sequence, and is encoded by the ORF corresponding to nucleotide bases 1457 to 1319 of SEQ ID NO:2.

In another embodiment, the protein or polypeptide of the present invention has an amino acid sequence corresponding to SEQ ID NO:26 as follows:

```
Met Ile Gln Leu Arg Phe Lys Ser Arg Ser Cys Val Ser Lys Arg Cys
    1 5 10 15
Phe Val Phe His Ser Ser Cys Val Ala Pro Asn Phe Ile Thr Val Lys
                          20 25 30
Asn Lys Asp Ala Ala Thr Leu Ser Arg
                  35 40
```

This protein or polypeptide has an estimated molecular weight of approximately 3 to 9 kilodaltons, and preferably about 4.67 kilodaltons, based on the deduced amino acid sequence, and is encoded by the ORF corresponding to nucleotide bases 1319 to 1193 of SEQ ID NO:2.

In another embodiment, the protein or polypeptide of the present invention has an amino acid sequence corresponding to SEQ ID NO:27 as follows:

```
Leu Cys Leu Val Arg Gln Pro Phe Cys Arg Ile Ser Ser Ala Leu Gly
    1 5 10 15
Met Gln Thr Leu His Cys Met Arg Ala Tyr Ala Arg Gly Tyr Thr His
                          20 25 30
Thr His Thr His Thr His Thr His Thr His Thr His Thr His Gly Thr
                  35 40 45
Arg Thr Ile Pro Asp Leu Ala Gly Gly Phe Asn Gly Glu Ser Thr His
          50 55 60
```

This protein or polypeptide has an estimated molecular weight of approximately 6 to 12 kilodaltons, and preferably about 7.30 kilodaltons, based on the deduced amino acid sequence, and is encoded by the ORF corresponding to nucleotide bases 1073 to 878 of SEQ ID NO:2.

In another embodiment, the protein or polypeptide of the present invention has an amino acid sequence corresponding to SEQ ID NO:28 as follows:

```
Leu Lys Ser Gln Glu Ile Leu Gln Arg Arg Ala Asp Lys Lys Cys Ser
    1 5 10 15
His Asn Pro Cys Ser Leu Pro Pro Thr His Met Tyr Thr Tyr Thr Tyr
                          20 25 30
Thr His Thr His Thr His Thr His Ala Pro Phe Cys Thr Pro Thr Ala
                  35 40 45
Gly Thr Gly Ser Val Thr Lys Met Leu Leu Pro Gly Ser His Leu Trp
          50 55 60
Gln Val Pro Gly Leu
  65
```

This protein or polypeptide has an estimated molecular weight of approximately 6 to 12 kilodaltons, and preferably about 7.87 kilodaltons, based on the deduced amino acid sequence, and is encoded by the ORF corresponding to nucleotide bases 749 to 539 of SEQ ID NO:2.

In another embodiment, the protein or polypeptide of the present invention has an amino acid sequence corresponding to SEQ ID NO:29 as follows:

```
His Gly Pro Leu Asn Cys Ser Ser Phe Val Phe Gln Ser Gln Gly Leu
    1 5 10 15
Phe Thr Pro Gln Gln Ser Ile Ser Gln Ser Lys Cys Leu Leu Asn Leu
                          20 25 30
Lys Leu Asn Gly His Leu Lys Val Gly Thr Ser Thr Pro Thr Val Glu
                  35 40 45
Glu Met Lys Leu Leu Ala Leu Leu Met Leu Ser Leu Gly Ser Arg Phe
          50 55 60
```

This protein or polypeptide has an estimated molecular weight of approximately 6 to 12 kilodaltons, and preferably about 7.30 kilodaltons, based on the deduced amino acid sequence, and is encoded by the ORF corresponding to nucleotide bases 539 to 344 of SEQ ID NO:2.

In another embodiment, the protein or polypeptide of the present invention has an amino acid sequence corresponding to SEQ ID NO:30 as follows:

```
Gly Trp Met Lys Phe Cys Pro Val Leu Ala Asn Ile Cys Arg Thr Ala
    1 5 10 15
Gln Ser Ala Phe Arg Glu Asn His Ala His Ile Leu Leu Asp Ser Ser
                          20 25 30
Ser Phe Gly Val Val Asn Thr Glu Ile Thr Glu Ser Leu Thr Pro Ile
                  35 40 45
Ala Gly Leu His Cys Ser Leu Thr Thr Arg Gly Arg
          50 55 60
```

This protein or polypeptide has an estimated molecular weight of approximately 5 to 11 kilodaltons, and preferably about 6.84 kilodaltons, based on the deduced amino acid sequence, and is encoded by the ORF corresponding to nucleotide bases 344 to 161 of SEQ ID NO:2.

Expression of the nucleic acid molecule conferring on a mammalian carcinoma cell an ability to undergo cell migration is induced in vivo by a growth factor. Suitable growth factors include, but are not limited to, HGF, insulin like growth factor (“ILGF”), and epidermal growth factor (“EGF”).

Activation of the isolated nucleic acid molecule conferring on a mammalian cell an ability to undergo migration can also be induced in vivo by activation of a tyrosine kinase protooncongene receptor. Suitable tyrosine kinase protooncongene receptors include, but are not limited to, c-Met, insulin receptor (“IR”), insulin like growth factor receptor (“ILGFR”), epidermal growth factor receptors (“EGFRs”), and platelet derived growth factor receptor (“PDGFR”). One mode of activation is through the binding of integrins to the tyrosine kinase protooncongene receptor. Suitable integrins include, but are not limited to, integrin αvβ5 and αvβ3. Thus, expression of the nucleic acid molecules of the present invention may be induced in vivo by activation of these various integrins.

The isolated nucleic acid molecule of the present invention confers on a human carcinoma cell an ability to undergo cell migration. The human carcinoma cell may be from various types of cells, including, without limitation, an ovary cell, a colon cell, an endometrial cell, a squamous cell, a uterus cell, a stomach cell, a lung cell, a breast cell, a prostate cell, a kidney cell, a rectum cell, a thyroid cell, a pancreas cell, a cervix cell, and intestine cell.

The isolated nucleic acid molecules of the present invention may also comprise a nucleotide sequence that is 99 percent homologous to SEQ ID NO:1 or SEQ ID NO:2, or a nucleotide sequence of at least 18 contiguous nucleic acid residues that hybridize to SEQ ID NO:1 or SEQ ID NO:2 under any of the following stringent conditions: (a) 6×SSC at 68° C.; (b) 5×SSC and 50% formamide 37° C.; or (c) 2×SSC and 40% formamide at 40° C.

Generally, suitable stringent conditions for nucleic acid hybridization assays or gene amplification detection procedures are as set forth above or as identified in Southern, “Detection of Specific Sequences Among DNA Fragments Separated by Gel Electrophoresis,” J. Mol. Biol., 98:503-17 (1975), which is hereby incorporated by reference in its entirety. For example, conditions of hybridization at 42° C. with 5×SSPE and 50% formamide with washing at 50° C. with 0.5×SSPE can be used with a nucleic acid probe containing at least 20 bases, preferably at least 25 bases or more preferably at least 30 bases. Stringency may be increased, for example, by washing at 55° C. or more preferably 60° C. using an appropriately selected wash medium having an increase in sodium concentration (e.g., 1×SSPE, 2×SSPE, 5×SSPE, etc.). If problems remain with cross-hybridization, further increases in temperature can also be selected, for example, by washing at 65° C., 70° C., 75° C., or 80° C. By adjusting hybridization conditions, it is possible to identify sequences having the desired degree of homology (i.e., greater than 80%, 85%, 90%, or 95%) as determined by the TBLASTN program (Altschul, S. F., et al., “Basic Local Alignment Search Tool,” J. Mol. Biol. 215:403-410 (1990), which is hereby incorporated by reference in its entirety) on its default setting.

The present invention also relates to nucleic acid molecules having at least 8 nucleotides (i.e., a hybridizable portion) of the nucleic acid molecules of SEQ ID NO:1 or SEQ ID NO:2. In other embodiments, the nucleic acid molecules have at least 12 (continuous) nucleotides, 18 nucleotides, 25 nucleotides, 50 nucleotides, 100 nucleotides, 150 nucleotides, or 200 nucleotides of a Mig-7 gene sequence, or a full-length Mig-7 gene coding sequence. The invention also relates to nucleic acid molecules hybridizable to or complementary to the foregoing sequences or their complements. In specific aspects, nucleic acid molecules are provided which comprise a sequence complementary to at least 10, 25, 50, 100, or 200 nucleotides or the entire coding region of a Mig-7 gene.

In a specific embodiment, a nucleic acid molecule which is hybridizable to a nucleic acid molecule of the present invention (e.g., having sequence SEQ ID NO:1 or SEQ ID NO:2, or an at least 10, 25, 50, 100, or 200 nucleotide portion thereof), or to a nucleic acid molecule encoding a derivative of a nucleic acid molecule of the present invention, under conditions of low stringency is provided. By way of example and not limitation, procedures using such conditions of low stringency are as follows (see also Shilo et al., PNAS USA 78:6789-6792 (1981), which is hereby incorporated by reference in its entirety): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

In another specific embodiment, a nucleic acid molecule which is hybridizable to a nucleic acid molecule of the present invention under conditions of high stringency is provided. By way of example and not limitation, procedures using such conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

Also suitable as an isolated nucleic acid molecule according to the present invention is an isolated nucleic acid molecule including at least 20 contiguous nucleic acid residues that hybridize to a nucleic acid having a nucleotide sequence of SEQ ID NO:1 or SEQ ID NO:2, or the complements of SEQ ID NO:1 or SEQ ID NO:2, under stringent conditions. Homologous nucleotide sequences can be detected by selectively hybridizing to each other. The term “selectively hybridizing” is used herein to mean hybridization of DNA or RNA probes from one sequence to the “homologous” sequence under stringent conditions which are characterized by a hybridization buffer comprising 2×SSC, 0.1% SDS at 56° C. (Ausubel et al., eds., Current Protocols in Molecular Biology, Vol. I, New York: Greene Publishing Associates, Inc. and John Wiley & Sons, Inc., p. 2.10.3 (1989), which is hereby incorporated by reference in its entirety). Another example of suitable stringency conditions is when hybridization is carried out at 65° C. for 20 hours in a medium containing 1M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μg/ml E. coli DNA. In one embodiment, the present invention is directed to isolated nucleic acid molecules having nucleotide sequences containing at least 20 contiguous nucleic acid residues that hybridize to the nucleic acid molecules of the present invention, including, SEQ ID NO:1 or SEQ ID NO:2 under stringent conditions including 50 percent formamide at 42° C.

Alternatively, or additionally, two nucleic acid sequences are substantially identical if they hybridize under high stringency conditions. By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.0 M Tris-Cl, pH 7.6, 1× Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing and 40 nucleotides or longer for in situ hybridization). The high stringency conditions used in these techniques are well known to those skilled in the art of molecular biology, and examples of them can be found, for example, in Ausubel et al., Current Protocols in Molecular Biology John Wiley & Sons, New York, N.Y., 1998, which is hereby incorporated by reference in its entirety.

The present invention further relates to compounds which specifically modulate or detect the expression of Mig-7 mRNA or protein, including but not limited to the nucleic acid molecule encoding Mig-7 and homologues, analogues, and deletions thereof, as well as antisense, ribozyme, triple helix, antibody, and polypeptide molecules and small inorganic molecules.

The present invention also relates to fragments of the proteins or polypeptides encoded by a nucleic acid molecule of the present invention.

Fragments of the proteins or polypeptides of the present invention can be produced by digestion of a fill-length elicitor protein with proteolytic enzymes like chymotrypsin or Staphylococcus proteinase A, or trypsin. Different proteolytic enzymes are likely to cleave the proteins or polypeptides of the present invention at different sites based on the amino acid sequence of the proteins or polypeptides. Some of the fragments that result from proteolysis may be active elicitors of resistance.

In another approach, based on knowledge of the primary structure of the protein or polypeptide, fragments of the genes encoding the proteins or polypeptides of the present invention may be synthesized by using the polymerase chain reaction technique together with specific sets of primers chosen to represent particular portions of the protein or polypeptide of interest. These then would be cloned into an appropriate vector for expression of a truncated peptide or protein.

Chemical synthesis can also be used to make suitable fragments. Such a synthesis is carried out using known amino acid sequences for the protein or polypeptide being produced. Alternatively, subjecting a full length protein or polypeptide of the present invention to high temperatures and pressures will produce fragments. These fragments can then be separated by conventional procedures (e.g., chromatography, SDS-PAGE).

Variants may also (or alternatively) be made, for example, by the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

The protein or polypeptide of the present invention is preferably produced in purified form (preferably at least about 80%, more preferably 90%, pure) by conventional techniques. Typically, the protein or polypeptide of the present invention is secreted into the growth medium host cells which express a functional type III secretion system capable of secreting the protein or polypeptide of the present invention. Alternatively, the proteins or polypeptides of the present invention are preferably produced in purified form by conventional techniques. To isolate the proteins or polypeptides, a protocol involving a host cell such as Escherchia coli may be used, in which protocol the E. coli host cell carrying a recombinant plasmid is propagated, homogenized, and the homogenate is centrifuged to remove bacterial debris. The supernatant is then subjected to sequential ammonium sulfate precipitation. The fraction containing the proteins or polypeptides of the present invention are subjected to gel filtration in an appropriately sized dextran or polyacrylamide column to separate the proteins or polypeptides. If necessary, the protein fraction may be further purified by high performance liquid chromatography (“HPLC”).

The DNA molecule encoding the proteins or polypeptides of the present invention can be incorporated in cells using conventional recombinant DNA technology. Generally, this involves inserting the DNA molecule into an expression system to which the DNA molecule is heterologous (i.e., not normally present). The heterologous DNA molecule is inserted into the expression system or vector in sense orientation and correct reading frame. The vector contains the necessary elements for the transcription and translation of the inserted protein-coding sequences. Thus, the present invention also relates to a DNA construct containing the nucleic acid of the present invention, which is operably linked to both a 5′ promoter and a 3′ regulatory region (i.e., transcription terminator) capable of affording transcription and expression of the encoded proteins or polypeptides of the present invention in host cells or host organisms.

The present invention also relates to an expression vector containing a DNA molecule encoding the proteins or polypeptides of the present invention. The nucleic acid molecules of the present invention may be inserted into any of the many available expression vectors using reagents that are well known in the art. In preparing a DNA vector for expression, the various DNA sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. The selection of a vector will depend on the preferred transformation technique and target host for transformation.

Suitable vectors for practicing the present invention include, but are not limited to, the following viral vectors such as lambda vector system gt11, gtWES.tB, Charon 4, and plasmid vectors such as pCMV, pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993)), pQE, pIH821, pGEX, pET series (Studier et al, “Use of T7 RNA Polymerase to Direct Expression of Cloned Genes,” Methods in Enzymology, 185:60-89 (1990), which is hereby incorporated by reference in its entirety), and any derivatives thereof. Any appropriate vectors now known or later described for genetic transformation are suitable for use with the present invention. Recombinant molecules can be introduced into cells via transformation, particularly transduction, conjugation, mobilization, or electroporation. The DNA sequences are cloned into the vector using standard cloning procedures in the art, as described by Maniatis et al., Molecular Cloning: A Laboratory Manual, Cold Springs Harbor, N.Y.: Cold Springs Laboratory, (1982), which is hereby incorporated by reference in its entirety.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology 68:473 (1979), which is hereby incorporated by reference in its entirety.

Promoters vary in their “strength” (i.e., their ability to promote transcription). For the purposes of expressing a cloned gene, it is generally desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the 17 phage promotor, lac promotor, trp promotor, recA promotor, ribosomal RNA promotor, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promotor or other E. coli promoters produced by recombinant DNA or other synthetic DNA techniques may be used to provide for transcription of the inserted gene.

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

In one aspect of the present invention, the nucleic acid molecule of the present invention is incorporated into an appropriate vector in the sense direction, such that the open reading frame is properly oriented for the expression of the encoded protein under control of a promoter of choice. This involves the inclusion of the appropriate regulatory elements into the DNA-vector construct. These include non-translated regions of the vector, useful promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used.

A constitutive promoter is a promoter that directs expression of a gene throughout the development and life of an organism.

An inducible promoter is a promoter that is capable of directly or indirectly activating transcription of one or more DNA sequences or genes in response to an inducer. In the absence of an inducer, the DNA sequences or genes will not be transcribed.

The DNA construct of the present invention also includes an operable 3′ regulatory region, selected from among those which are capable of providing correct transcription termination and polyadenylation of mRNA for expression in the host cell of choice, operably linked to a DNA molecule which encodes for a protein of choice.

The vector of choice, promoter, and an appropriate 3′ regulatory region can be ligated together to produce the DNA construct of the present invention using well known molecular cloning techniques as described in Sambrook et al., Molecular Cloning: A Laboratory Manual, Second Edition, Cold Spring Harbor Press, NY (1989), and Ausubel, F. M. et al. Current Protocols in Molecular Biology, New York, N.Y: John Wiley & Sons,. (1989), which are hereby incorporated by reference in their entirety.

Once the DNA construct of the present invention has been prepared, it is ready to be incorporated into a host cell. Accordingly, another aspect of the present invention relates to a method of making a recombinant cell. Basically, this method is carried out by transforming a host cell with a DNA construct of the present invention under conditions effective to yield transcription of the DNA molecule in the host cell. Recombinant molecules can be introduced into cells via transformation, particularly transduction, conjugation, mobilization, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable host cells include, but are not limited to, bacteria, virus, yeast, mammalian cells, insect, plant, and the like.

The present invention also relates to a recombinant DNA expression system having an expression vector into which is inserted an isolated nucleic acid molecule conferring on a mammalian carcinoma cell an ability to undergo cell migration. The nucleic acid molecule may be heterologous to the expression vector or inserted into the vector in proper sense orientation and correct reading frame.

The present invention also relates to a host cell incorporating an isolated nucleic acid molecule conferring on a mammalian carcinoma cell an ability to undergo cell migration. In one embodiment, the isolated nucleic acid molecule is heterologous to the host cell.

The present invention also relates to an antisense oligonucleotide of at least 8 contiguous nucleic acid residues targeted to a nucleic acid molecule conferring on a mammalian carcinoma cell an ability to undergo cell migration. The antisense oligonucleotide may hybridize to an isolated nucleic acid molecule that codes for a mammalian migration inducting gene (e.g., Mig-7), has a nucleotide sequence of SEQ ID NO:1 or SEQ ID NO:2, has a nucleotide sequence that is 99 percent homologous to SEQ ID NO:1 or SEQ ID NO:2, or has a nucleotide sequence of at least 18 contiguous nucleic acid residues that hybridize to SEQ ID NO:1 or SEQ ID NO:2 under the following stringent conditions: (a) 6×SSC at 68° C.; (b) 5×SSC and 50% formamide 37° C.; or (c) 2×SSC and 40% formamide at 40° C. In one embodiment, the antisense oligonucleotide may hybridize to nucleotides 275 to 292 or nucleotides 324 to 343 of SEQ ID NO:1, or to nucleotides 760 to 777 or nucleotides 809 to 828 of SEQ ID NO:2.

The present invention also relates to a method for inhibiting expression, in a subject, of a nucleic acid molecule conferring on a human carcinoma cell an ability to undergo cell migration. This method involves administering to the subject an inhibitor capable of blocking binding of a growth factor to at least one receptor for the growth factor under conditions effective to inhibit the expression of the nucleic acid molecule. The growth factor may be HGF, ILGF, and EGF, and the receptor can be c-Met, IR, ILGFR, EGFR, PDGFR, integrin αvβ5, or integrin αvβ3. Alternatively, the inhibitor binds to the growth factor, or to the receptor.

The present invention also relates to a protein or polypeptide encoded by a nucleic acid molecule conferring on a mammalian carcinoma cell an ability to undergo cell migration. In one aspect, the protein or polypeptide comprises an amino acid sequence corresponding SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, or SEQ ID NO:30.

The present invention also relates to an isolated antibody or binding portion thereof raised against a protein or polypeptide encoded by a nucleic acid molecule conferring on a mammalian carcinoma cell an ability to undergo cell migration. A suitable protein or polypeptide used to prepare the antibody or portion thereof includes, but is not limited to, one comprising an amino acid sequence corresponding to SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, or SEQ ID NO:30. In one aspect, the antibody is monoclonal or polyclonal.

Suitable antibodies can be monoclonal or polyclonal.

Monoclonal antibody production may be effected by techniques which are well-known in the art. Basically, the process involves first obtaining immune cells (lymphocytes) from the spleen of a mammal (e.g., mouse) which has been previously immunized with the antigen of interest (i.e., the protein or peptide of the present invention) either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference in its entirety.

Mammalian lymphocytes are immunized by in vivo immunization of the animal (e.g., a mouse) with one of the proteins or polypeptides of the present invention. Such immunizations are repeated as necessary at intervals of up to several weeks to obtain a sufficient titer of antibodies. The virus is carried in appropriate solutions or adjuvants. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (PEG) or other fusing agents (See Milstein and Kohler, Eur. J. Immunol. 6:511 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth and to have good fusion capability. Many such cell lines are known to those skilled in the art, and others are regularly described.

Procedures for raising polyclonal antibodies are also well known. Typically, such antibodies can be raised by administering one of the proteins or polypeptides of the present invention subcutaneously to New Zealand white rabbits which have first been bled to obtain pre-immune serum. The antigens can be injected at a total volume of 100 μl 1 per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbitol 150 mg/Kg IV. This and other procedures for raising polyclonal antibodies are disclosed in E. Harlow, et. al., editors, Antibodies: A Laboratory Manual (1988), which is hereby incorporated by reference in its entirety.

In addition to utilizing whole antibodies, the processes of the present invention encompass use of binding portions of such antibodies. Such antibody fragments can be made by conventional procedures, such as proteolytic fragmentation procedures, as described in J. Goding, Monoclonal Antibodies: Principles and Practice, pp. 98-118 (N.Y. Academic press 1983), which is hereby incorporated by reference in its entirety.

The present invention also relates to a method for inhibiting production, in a subject, of a protein or polypeptide encoded by a nucleic acid molecule conferring on a carcinoma cell an ability to undergo cell migration. This method involves administering to the subject the antisense oligonucleotide of the present invention, which is complementary to a target portion of the nucleic acid molecule, under conditions effective to inhibit production of the protein or polypeptide.

The present invention also relates to a method for inhibiting metastasis of a carcinoma cell in a subject. This method involves administering to the subject the antisense oligonucleotide of the present invention, which is complementary to a target portion of a nucleic acid molecule conferring on a carcinoma cell an ability, in vivo, to undergo cell migration under conditions effective to inhibit metastasis of the carcinoma cell. This method may involve infusing or injecting the antisense oligonucleotide into the subject, as appropriate and in accordance with well known procedures in the art.

The present invention also relates to a method for inhibiting metastasis of a carcinoma cell in ahuman subject. This method involves administering to the subject an inhibitor capable of blocking the binding of a growth factor to at least one receptor for the growth factor under conditions effective to inhibit metastasis of the carcinoma cell. In one aspect of this method the growth factor is HGF, ILGF, or EGF, and the receptor is c-Met, IR, ILGFR, EGFR, PDGFR, integrin αvβ5, or integrin αᵥβ₃. Alternatively, the inhibitor binds to the hepatocyte growth factor or to the receptor.

The present invention also involves a method for inhibiting migration of a carcinoma cell in a subject. This method involves administering to the subject the antisense oligonucleotide of the present invention, which is complementary to a target portion of a nucleic acid molecule conferring on a carcinoma cell an ability, in vivo, to undergo cell migration, under conditions effective to inhibit migration of the carcinoma cell.

The present invention also relates to a method for inhibiting migration of a carcinoma cell in a subject. This method involves administering to the subject an inhibitor capable of blocking binding of a growth factor to at least one receptor for the growth factor under conditions effective to inhibit migration of the carcinoma cell. In one aspect of this method the growth factor is HGF, ILGF, or EGF, and the receptor is c-Met, IR, ILGFR, EGFR, PDGFR, integrin αvβ5, or integrin αᵥβ₃. Alternatively, the inhibitor binds to the hepatocyte growth factor or to the receptor.

The present invention also relates to a method for detecting the presence of a migrating carcinoma cell in a sample of a subject's tissue or body fluids. This method involves (1) providing a protein or polypeptide as an antigen, where the protein or polypeptide is encoded by a nucleic acid molecule conferring on a mammalian carcinoma cell an ability to undergo cell migration; (2) contacting the sample with the antigen; and (3) detecting any reaction which indicates that the migrating carcinoma cell is present in the sample using an assay system. The assay system may be an enzyme-linked immunosorbent assay, a radioimmunoassay, a gel diffusion precipitin reaction assay, an immunodiffusion assay, an agglutination assay, a fluorescent immunoassay, a protein A immunoassay, an immunoelectrophoresis assay, or other relevant detection techniques well known in the art (see de Groot et al., “Design, Synthesis, and Biological Evaluation of a Dual Tumor-Specific Motive Containing Integrin-Targeted Plasmin-Cleavable Doxorubicin Prodrug,” Molecular Cancer Therapeutics 1(11):901-911 (2002). In one embodiment of this method, an antibody to Mig-7 is linked to a doxorubicin prodrug in order to make the detection method cancer cell specific.

The present invention also relates to a second method for detecting the presence of a migrating carcinoma cell in a sample of a subject's tissue or body fluids. This method involves (1) providing an antibody or binding portion thereof raised against a protein or polypeptide encoded by a nucleic acid molecule conferring on a mammalian carcinoma cell an ability to undergo cell migration; (2) contacting the sample with the antibody or binding portion thereof; and (3) detecting any reaction which indicates that the migrating carcinoma cell is present in the sample using an assay system. The assay system may be an enzyme-linked immunosorbent assay, a radioimmunoassay, a gel diffusion precipitin reaction assay, an immunodiffusion assay, an agglutination assay, a fluorescent immunoassay, a protein A immunoassay, or an immunoelectrophoresis assay.

The present invention also relates to a third method for detecting the presence of a migrating carcinoma cell in a sample of a subject's tissue or body fluids. This method involves (1) providing a nucleotide sequence as a probe in a nucleic acid hybridization assay, where the nucleotide sequence is a nucleic acid molecule conferring on a mammalian carcinoma cell an ability to undergo cell migration; (2) contacting the sample with the probe; and (3) detecting any reaction which indicates that the migrating carcinoma cell is present in the sample.

The present invention also relates to a fourth method for detecting the presence of a migrating carcinoma cell in a sample of a subject's tissue or body fluids. This method involves (1) providing a nucleotide sequence as a probe in a gene amplification detection procedure, where the nucleotide sequence is a nucleic acid molecule conferring on a mammalian carcinoma cell an ability to undergo cell migration; (2) contacting the sample with the probe; and (3) detecting any reaction which indicates that the migrating carcinoma cell is present in the sample.

The present invention further relates to a first method of inhibiting the migration of placental cells into a blood stream of a mammalian subject. This method can be useful in treated or inhibiting the manifestation of certain autoimmune diseases in female mammalian subjects, including, without limitation, in humans. This method involves administering to the mammalian subject the subject antisense oligonucleotide complementary to a target portion of a nucleic acid molecule conferring on the placental cells an ability, in vivo, to undergo cell migration under conditions effective to inhibit migration of said placentals cells into the blood stream.

The present invention also relates to a second method of inhibiting the migration of placental cells into a blood stream of a mammalian subject. This method involves administering to the mammalian subject an inhibitor capable of blocking binding of a growth factor to at least one receptor for the growth factor under conditions effective to inhibit migration of said placental cells. In one aspect of this method the growth factor is HGF, ILGF, or EGF, and the receptor is c-Met, IR, ILGFR, EGFR, PDGFR, integrin αvβ5, or integrin αvβ3. Alternatively, the inhibitor binds to the hepatocyte growth factor or to the receptor.

The present invention also relates to using a protein or polypeptide of the present invention derived from SEQ ID NO:1 or SEQ ID NO:2, including, without limitationor, proteins or polypeptides comprising an amino acid sequence of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, or SEQ ID NO:30, to stimulate and/or activate immune cells ex-vivo or in vivo.

The present invention also relates to a method of inducing the establishment of anchoring villi and blood supply to a mammalian fetus. This method involves transducing the ectopic expression of the nucleic acid molecule of the present invention using a suitable expression vector into cytotrophoblast cells or precursors thereof, under conditions effective to induce the establishment of anchoring villi and blood supply to a mammalian fetus.

The present invention further relates to a method of transgenically expressing the nucleic acid molecule of the present invention in a mammalian cell. This method involves cloning the nucleic acid molecule of the present invention into a suitable expression vector and transfecting the vector into a mammalian cell using suitable means of transfection, under conditions effective to transgenically express the nucleic acid molecule in a mammalian cell. Suitable means of transfection include, but are not limited to, electroporation, lipophilic reagent, and calcium chloride. In one embodiment, the method involves transgenic expression of the protein or polypeptide of the present invention (i.e., inducing expression via an expression vector in cells), especially in placental cytotrophoblast cells, which may be deficient in expression of the protein or polypeptide of the present invention and where more invasion would be desired to prevent eclampsia, placental failure, or lack of adequate blood supply to the fetus.

The present invention also relates to a method for detecting the presence of fetal cytotrophoblast cells in a sample of a subject's tissue or body fluids. This method involves providing a nucleotide sequence corresponding to the nucleic acid molecule of the present invention as a probe in a detection assay, contacting the sample with the probe, and detecting any reaction which indicates that fetal cytotrophoblast cells are present in the sample. Suitable detection assays include, but are not limited to, nucleic acid hybridization assays and gene amplification detection procedures.

### EXAMPLES

### Example 1

### HGF and Ligation of αvβ5 Integrin Induce a Novel, Cancer Cell-Specific Gene Expression Required for Cell Migration

Hepatocyte growth factor (HGF), a cytokine involved in tumorigenesis and most metastases, initiates cell migration by binding to the protooncogene c-Met receptor. In epithelial carcinoma cells, c-Met activation causes the breakdown of E-cadherin cell-cell contacts leading to cell spreading. While the breakdown of E-cadherin contacts is immediate, HGF-induced migration requires transcription. To test the hypothesis that this de novo mRNA synthesis includes cancer cell-specific transcripts, subtraction hybridization was performed to isolate HGF-induced transcripts from an endometrial epithelial carcinoma cell line, RL95-2 (RL95), known to migrate but not to proliferate with HGF treatment. Mig-7 cDNA is induced by HGF in endometrial epithelial carcinoma cell lines RL95 and HEC-1A before migration ensues. Ovarian, oral squamous cell, and colon metastatic tumors but not normal tissues express Mig-7. HGF did not induce Mig-7 in normal, primary endometrial epithelial cells. In addition, blocking antibodies to αvβ5 integrin inhibited HGF induction of Mig-7 and another isolated clone in RL95 cells. Most importantly, Mig-7 specific antisense oligonucleotides inhibited migration of RL95 cells in vitro. These results are the first to demonstrate that Mig-7 expression may be used as a cancer cell-specific target to inhibit cell migration.

To investigate cancer cell specificity of genes induced by HGF, a functional genomic approach was used. A PCR-based subtraction hybridization was employed to compare cDNAs from an early time point of HGF treatment with cDNAs from untreated carcinoma cells as opposed to long term HGF treatment or established migration. The rationale was that cell specific gene transcription is more typical at early time points, rather than immediate or late, in a signal transduction pathway. The endometrial epithelial carcinoma cell line, RL95-2 (RL95), shown to express c-Met but not HGF (Moghul et al., “Modulation of c-MET Proto-Oncogene (HGF Receptor) mRNA Abundance by Cytokines and Hormones: Evidence for Rapid Decay of the 8 kb c-MET Transcript,” Oncogene 9:2045-2052 (1994), which is hereby incorporated by reference in its entirety) was used. This cell line was also shown to migrate but not to proliferate or undergo tubulogenesis after treatment with HGF in vitro (Bae-Jump et al., “Hepatocyte Growth Factor (HGF) Induces Invasion of Endometrial Carcinoma Cell Lines In Vitro,” Gynecologic Oncology 73:265-272 (1999), which is hereby incorporated by reference in its entirety). The characterization, integrin involvement, and function of HGF-induced cDNA was studied.

### Example 2

### Cell Culture and Reagents

Human cell lines were used. The endometrial carcinoma cell line, RL952 (ATCC), was cultured in DMEM:Ham's F12 (1:1) media (Gibco) supplemented with 10 mM HEPES (Sigma), 0.005 mg/ml insulin (Sigma), 2.0 g/L NaHCO₃, and 10% FCS at 37° C., 5% CO₂. Because cancer cells can lay down and modify ECM differently than do normal cells (Emoto et al., “Annexin II Overexpression Correlates with Stromal Tenascin-C Overexpression: A Prognostic Marker in Colorectal Carcinoma,” Cancer 92:1419-1426 (2001); Euer et al., “Identification of Genes Associated with Metastasis of Mammary Carcinoma in Metastatic Versus Non-Metastatic Cell Lines,” Anticancer Research 22:733-740 (2002); Matsuyama et al., “Comparison of Matrix Metalloproteinase Expression Between Primary Tumors With or Without Liver Metastasis in Pancreatic and Colorectal Carcinomas,” Journal of Surgical Oncology 80:105-110 (2002), which are hereby incorporated by reference in their entirety), the cells were plated for four days in the absence of exogenous extracellular matrix (ECM) to allow time for ECM deposition and modification. All cell lines were cultured for four days prior to serum starvation at 50-60% confluency. Before treatment with 50 ng/ml HGF (Sigma or R&D Systems), cells were cultured in serum free, phenol free media for 48-50 hours with 100 ng/ml IL-6 to stabilize c-Met expression (Moghul et al., “Modulation of c-MET Proto-Oncogene (HGF Receptor) mRNA Abundance by Cytokines and Hormones: Evidence for Rapid Decay of the 8 kb c-MET Transcript,” Oncogene 9:2045-2052 (1994), which is hereby incorporated by reference in its entirety). HEC-LA endometrial carcinoma cells (ATCC) were cultured in McCoy's Sa medium with 10% FCS at 37° C. and 5% CO₂. Integrin blocking antibodies, β1 antibody (GS6), αvβ5 antibody (P1F6), and αvβ6 antibody (10D5) were all purchased from Chemicon and used at 8 μg/ml. Cells were cultured in respective blocking antibody for 30 minutes before the addition of HGF.

### Example 3

### Primary Endometrial Epithelial Cell Isolation

Under IRB approval, normal endometrial tissue (functionalis region) was obtained from three individuals undergoing hysterectomy for reasons other than cancer. Primary endometrial epithelial cells were isolated as previously described (Classen-Linke et al., “Establishment of a Human Endometrial Cell Culture System and Characterization of its Polarized Hormone Responsive Epithelial Cells,” Cell Tissue Research 287:171-185 (1997), which is hereby incorporated by reference in its entirety). Briefly, sections of normal functionalis were immediately placed in ice-cold DMEM:F12 media with 10% FCS, minced with sterile scissors under sterile conditions and treated with 0.46% Type I collagenase A (125 U/mg, Sigma), 1% penicillin/streptomycin (Gibco BRL) and incubated for one hour at 37° C. in a shaking water bath. Stromal cells were separated from glandular epithelium by filtration with a 250 μm sterile mesh followed by a second filtration through a 36 μm sterile mesh, which captured the clumps of glandular, and luminal epithelium. These cells were then washed 2× with prewarmed medium followed by centrifugation at 75 g for 10 min. The cell pellet was resuspended in medium and cell density was determined with a hemocytometer. Cells at 5×10⁵ per individual were pooled and plated in a 10 cm plate. Cells were determined to be epithelial by their cuboidal morphology and by their expression of c-Met, which is not expressed by stromal cells (Sugawara et al., “Hepatocyte Growth Factor Stimulated Proliferation, Migration, and Lumen Formation of Human Endometrial Epithelial Cells In Vitro,” Biology of Reproduction 57:936-942 (1997), which is hereby incorporated by reference in its entirety). These cells were then treated as described previously for cell lines.

### Example 4

### Isolation of RNA, cDNA Library Synthesis, and SSH

RNA-STAT (Tel-Test) was used according to manufacturer's directions previously described (Chomczyncki et al., “Single-Step Method of RNA Isolation by Acid Guanidinium Thiocynate-Phenol-Chloroform Extraction,” Analytical Biochemistry 162:156-159 (1987), which is hereby incorporated by reference in its entirety) to isolate total RNA. Synthesis of cDNA was performed according to manufacturer's protocol using the SMART™ PCR cDNA synthesis kit (Clontech) that enriches for full-length cDNAs. For cDNA synthesis, 1 μg of total RNA each from untreated and treated (HGF 50 ng/ml for 6 hours) RL95 cells was used. For SSH, the PCR-Select™ kit (Clontech) was used according to manufacturer's instructions with all controls.

### Example 5

### Rapid Amplification of cDNA Ends (RACE)

The Mig-7 5′ transcript region was isolated by RACE using the FirstChoice RLM-RACE kit (Ambion) according to manufacturer's directions. RLM-RACE was designed to isolate full-length cDNA ends and not partial cDNA from degraded RNA (Shaefer, B., “Revolution in Rapid Amplification of cDNA Ends: New Strategies for Polymerase Chain Reaction Cloning of Full-Length cDNA Ends,” Analytical Biochemistry 227:255-273 (1995), which is hereby incorporated by reference in its entirety). Briefly, 10 μg of total RNA isolated from RL95 cells treated with HGF for twelve hours or control RNA was treated with calf intestinal phosphotase (CIP) to remove the 5′-PO₄ from degraded mRNA, rRNA, tRNA and DNA. After removal of CIP by phenol/chloroform extraction, RNA was treated with tobacco acid phosphotase (TAP) to remove the cap from full-length mRNA leaving one phosphate group at the 5′ end. An RNA adapter sequence (5′-GCU GAU GGC GAU GAA UGA ACA CUG CGU UUG CUG GCU UUG AUG AAA-3′) (SEQ ID NO:31) was ligated by using T4 RNA ligase. Quality of RNA was predetermined by formaldehyde agarose gel electrophoresis and deemed high quality based on distinct 18s and 28s ribosomal bands. cDNA was prepared from denatured CIP, TAP treated RNA using the Thermoscript™ RT System (Life Technologies) and priming with oligo dT at 58° C. Nested PCR was performed using outer primers to the adapter (5′-GCT GAT GGC GAT GAA TGA ACA CTG-3′) (SEQ ID NO:32) and to Mig-7 (5′-CCT CGG TCT GTC TTC TTG ACT TTG T-3′) (SEQ ID NO:33) followed by a second PCR reaction using 2 μl of the first PCR reaction and inner primers to the adapter (5′-CGC GGA TCC GAC ACT COT TTG CTG GCT TTG ATG-3′) (SEQ ID NO:34) and to Mig-7 (5′-CAG ATG GCC COT GAT GAA TC-3′) (SEQ ID NO:35). Products were run on a 1% agarose gel along with control products. The negative control of RL95 RNA not treated with TAP and carried through the rest of the RACE steps was negative for any product. RACE PCR products were gel purified.

### Example 6

### Clone Isolation and Sequencing

After SSH or RACE, the PCR products were ligated into the T/A Cloning vector PCRII (Invitrogen). Top 10F′ E. coli (Invitrogen) were transformed and grown, colonies were selected from ampicillin (50 μg/ml) LB agar plates. Seven colonies and eight colonies were isolated from the SSH and RACE experiments, respectively, and screened initially by PCR for inserts. Plasmid DNA was isolated and sent to the Texas Tech University Health Science Center Biotechnology Core for sequencing. Sequences were analyzed by GCG, Vector NTI, and Labonweb programs to determine sequence homologies, overlap, and motifs.

### Example 7

### Northern Blotting, Isotope Labeling of Probe and Densitometry

RNA (20 μg/sample) was elctrophoresed in 1% formaldehyde agarose gels. The separated RNA was transferred to positively charged membranes (Boehringer-Mannheim) by capillary action as previously described (Lindsey et al., “Pem: A Testosterone- and LH-Regulated Homeobox Gene Expressed in Mouse Sertoli Cells and Epididymis,” Developmental Biology 179:471-484 (1996), which is hereby incorporated by reference in its entirety). After transfer, the membrane was crosslinked by ultraviolet irradiation (Ultra-Lum) and stained with methylene blue to directly evaluate the transfer and loading of RNA in each lane. Blots were then destained followed by prehybridization and hybridization with ExpressHyb (Clontech) for 30 mins and one hour, respectively, at 68° C. Random oligomer-primed, ³²P-labeled Mig-7, clone 34 (both at 5×10⁶ cpm/ml) or actin (at 5×10⁵) specific cDNA was used as probe. After stringent washings, the membrane was exposed to film for the indicated times at −70° C.

Densitometry was performed using the Bio-Rad GS-700 densitometer and Molecular Analyst® Software (Bio-Rad Laboratories). The signal intensity of each Mig-7 specific band was normalized to the respective 18s ribosomal or actin band intensity for each sample.

### Example 8

### Relative RT-PCR

Under IRB approval, human tissue was obtained from the co-operative Human Tissue Network. Total RNA was isolated using the acid guandinium thiocyante, phenol-chloroform extraction method (Chomczyncki et al., “Single-Step Method of RNA Isolation by Acid Guanidinium Thiocynate-Phenol-Chloroform Extraction,” Analytical Biochemistry 162:156-159 (1987), which is hereby incorporated by reference in its entirety) as previously described (Lindsey et al., “Pem: A Testosterone- and LH-Regulated Homeobox Gene Expressed in Mouse Sertoli Cells and Epididymis,” Developmental Biology 179:471-484 (1996), which is hereby incorporated by reference in its entirety). After quantitation by UV spectrophotometer at 260 and 280 nm wavelengths, first strand synthesis, reverse transcription (RT) was performed using 1 μg of total RNA that had been DNased (DNA-free™, Ambion). The Thermoscript RT kit (BRL Lifetech) was used with random hexamers as primers according to manufacturer's directions at an incubation of 58° C. for 50 minutes. PCR of 1 μl of the RT reaction was performed using a thermal cycler (MJResearch, Inc.) SuperTaq Plus™ (Ambion), buffer containing 1.5 mM MgCl₂ and the following primer sets: Mig-7 forward 5′-GAC AAA GTC AAG AAG ACA GAC C-3′ (SEQ ID NO:36), Mig-7 reverse 5′-ACC CCT CTA TTT GAT ATC TCA CA-3′ (SEQ ID NO:37), c-Met forward, 5′-ATC CAG AAT GTC ATT CTA CA-3′ (SEQ ID NO:38), c-Met reverse, 5′-TGA TCT GGG AAA TAA GAA GA-3′ (SEQ ID NO:39) and 18s primer pair set (Classic II, Ambion). Samples were prepared on ice and subjected to a hot start at 94° C. for 2 minutes followed by 40 cycles (Mig-7 primers) of: 94° C. for 30 seconds, 55° C. for 30 seconds (50° C. for c-Met primers, and 57° C. for 18s primers), 68° C. for 33 seconds and a final extension for 5 minutes. Reactions with c-Met primers and 18s primers were cycled 34 and 31 times, respectively. These numbers of cycles for each primer set were determined to be mid-linear range of amplification by removing replicate PCR reactions at alternate cycles starting at cycle 15 through cycle 50, running the reactions on a 1.5% agarose gel containing ethidium bromide and performing densitometry. PCR products were confirmed by Southern blot (Mig-7 specific cDNA probe) or by subcloning and sequencing c-Met.

### Example 9

### Antisense Treatment and Migration Assay

This study was blinded in that one technician prepared the oligonucleotides and the identity of the oligonucleotides was by number only. Another technician who did not know the labeling scheme of the oligonucleotides counted the cell migration assay and migrated cells. RL95 cells were plated in six well plates at 10% confluency. After culturing for four days, cells were treated with serum-free, phenol-free DMEM:F12 for 48 hours after which a wounded area was created in each well with a sterile pipette tip. Each oligo in FuGene (as directed by Roche) was used at lug/ml of serum- and phenol-free media. After 15 minutes, 50 ng/mil of HGF was added. Migrated cells were counted 24 hours later. HGF treated RL95 cells without any oligonucleotides were used as positive migration control. The Mig-7 specific antisense oligonucleotide sequences are: 5′-GCA CTA TGG GCT TAT GGG-3′ (SEQ ID NO:40) (antisense to nucleotides 275-292 of SEQ ID NO:1 or antisense to nucleotides 760-777 of SEQ ID NO:2), and 5′-GCA TCT ACT TGC TGC CAT GG-3′ (SEQ ID NO:41) (antisense to nucleotides 324-343 of SEQ ID NO:1 or antisense to nucleotides 809-828 of SEQ ID NO:2). The irrelevant oligonucleotide was 5′-GGG TAT TCG GGT ATT ACG-3′ (SEQ ID NO:42). This experiment was performed in triplicate wells for each treatment. Three fields of view were counted in the scraped “wound” area per well then averaged for that well. The statistical analyses were performed using the student's t-test and Microsoft Excel software program.

### Example 10

### Isolation of Mig-7 cDNA and Sequence Analyses

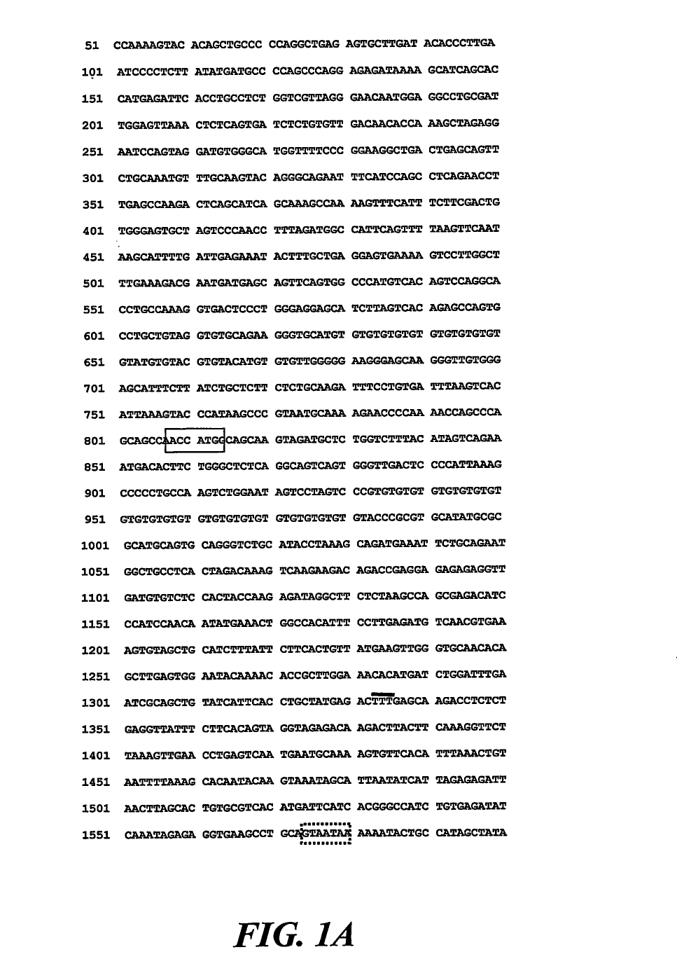

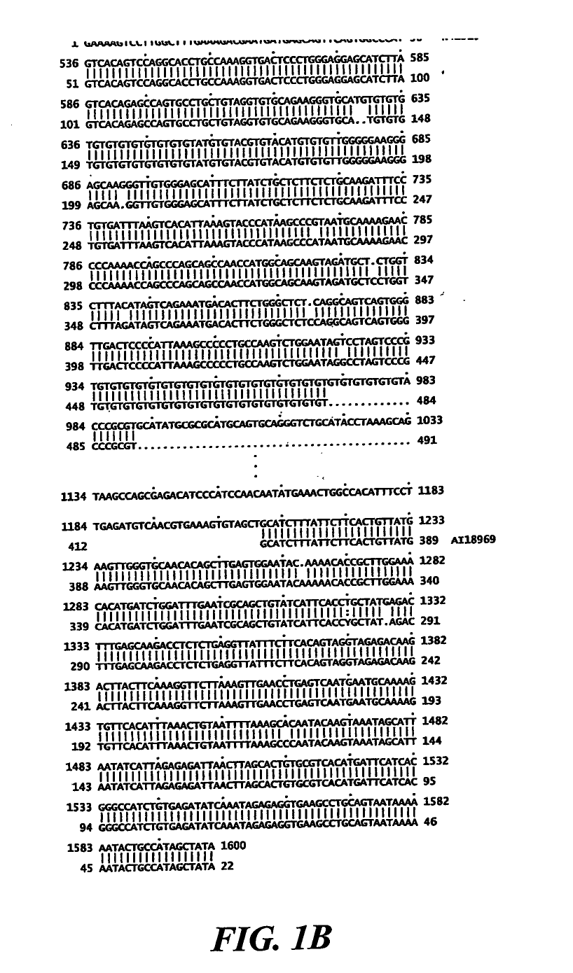

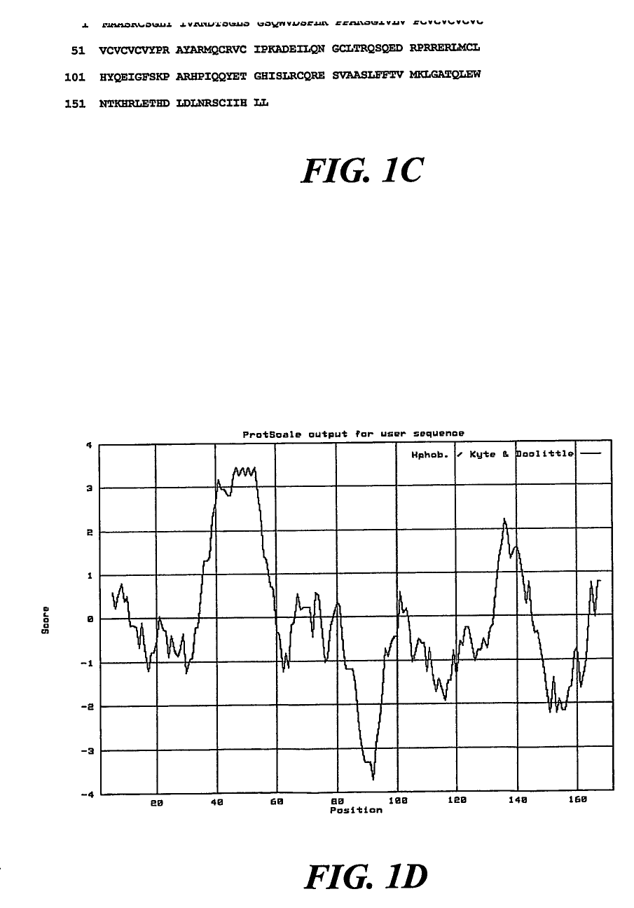

SSH is a PCR-based and highly sensitive method of subtraction hybridization used to isolate lowly expressed genes (Diatchenko et al., “Suppression Subtractive Hybridization: A Method for Generating Differentially Regulated or Tissue-Specific cDNA Probes and Libraries,” Proceedings of the National Academy of Science 93:6025-6030 (1996), which is hereby incorporated by reference in its entirety) such as early genes. RL95 cells were specifically treated for 2.5 hours before isolating total RNA for cDNA synthesis. cDNA from untreated cells was used to subtract out non-induced transcripts. This time point was chosen for study, because HGF-induced genes had not been determined before migration ensued. After performing rapid identification of cDNA ends (RACE) and database analyses, the sequence of one clone we call Mig-7 (FIG. 1A) was identified homologous to two existing ESTs, N41315 to Mig-7 5′ and AI18969 to Mig-7 3′ (FIG. 1B) but not to any sequences of known function. Of note, these two ESTs were isolated from 8-9 weeks of gestation human placenta, a tissue consistent with an invasive phenotype (Dokras et al., “Regulation of Human Cytotrophoblast Morphogenesis Hepatocyte Growth Factor/Scatter Factor,” Biology of Reproduction 65:1278-1288 (2001), which is hereby incorporated by reference in its entirety) and dependent on HGF for its development (Uehara et al., “Placental Defect and Embryonic Lethality in Mice Lacking Hepatocyte Growth Factor/Scatter Factor,” Nature 373:702-705 (1995), which is hereby incorporated by reference in its entirety). In addition, Mig-7 sequences were found to be homologous to regions of chromosome 1 (accession numbers AL512488 and AX261960).

It is proposed that the Mig-7 transcript is translated because it is polyadenylated and because it encodes a translation start site within the context of a KOZAK consensus sequence (FIG. 1A) (Kozak, M., “The Scanning Model for Translation: An Update,” Journal of Cell Biology 108:229-241 (1989), which is hereby incorporated by reference in its entirety). Mig-7 protein (FIG. 1C) homology searches show no significant homology to any banked sequences. There is a repeat of thymidines and guanosines that encodes an eight valine-cysteine dipeptide repeat region. Only one protein, Q300 (X52164), in the database had a similar seven valine-cysteine dipeptide repeat. Q300 is induced by the SV40 T antigen and is predicted to be a membrane protein in the dipeptide repeat region as is also predicted by the Mig-7 hydrophobicity plot (FIG. 1D). Therefore, these results strongly suggest that a novel, HGF-induced gene has been isolated.

### Example 11

### Confirmation of HGF-Induction in RL95 and HEC-1A Cell Lines

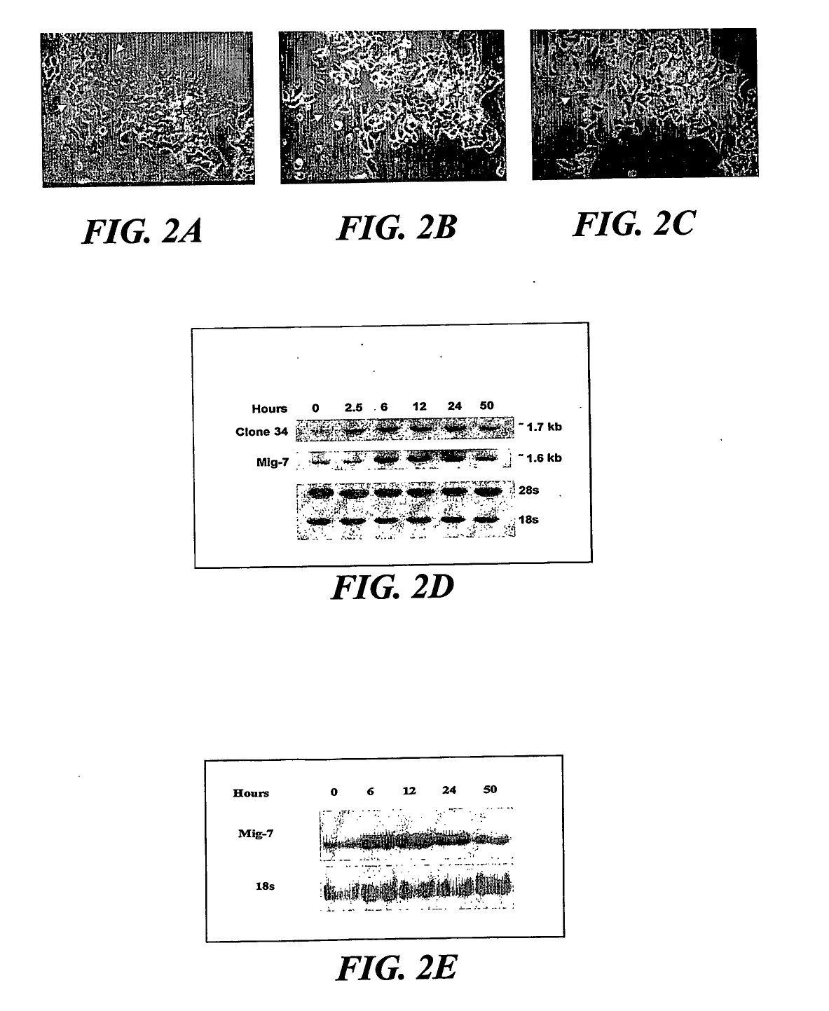

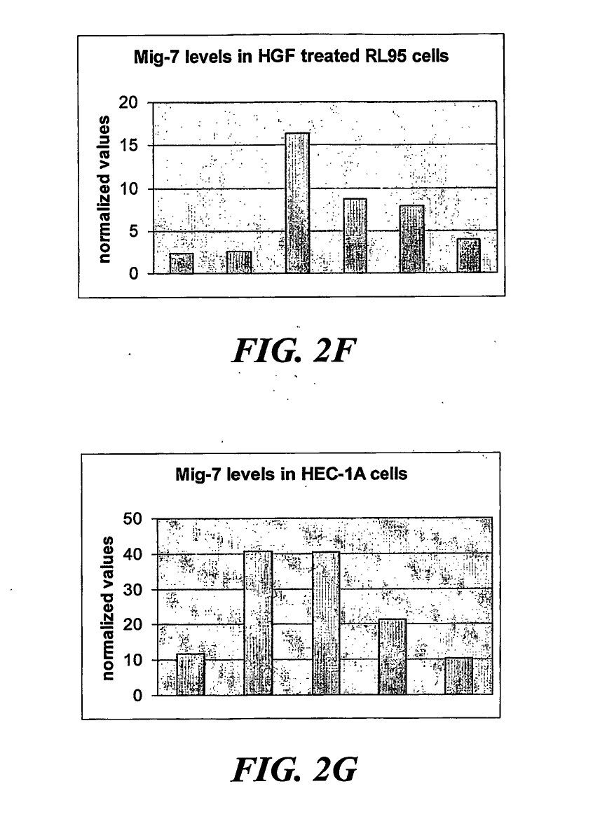

HGF reproducibly induced Mig-7 mRNA (middle tier of Northern blot FIGS. 2A-2C and 2F) by seven fold in RL95 cells and by four fold in another endometrial carcinoma cell line, HEC1A (FIGS. 2D and 2G), subjected to serum- and phenol-free media for two days prior to SF treatment. A time course revealed that the highest levels of Mig-7 expression occurred in RL95 cells 6 hours after a single dose (50 ng/ml) of SF (FIGS. 2A-2C). Mig-7 expression levels rapidly decreased returning to near basal levels by 50 hours. Low levels of Mig-7 expression were detected at 2.5 hours after treatment but are likely due to culturing cells for only 48 hours in the absence of serum since serum contains HGF and can trigger Mig-7 expression. The higher basal level of Mig-7 expression in HEC-LA correlates to the more invasive ability of this cell line as compared to RL95 cells (Bae-Jump et al., “Hepatocyte Growth Factor (HGF) Induces Invasion of Endometrial Carcinoma Cell Lines In Vitro,” Gynecologic Oncology 73:265-272 (1999), which is hereby incorporated by reference in its entirety).

For comparison, another clone, 34, isolated by SSH at the same time as Mig-7, was also shown to be reproducibly induced by HGF and is induced at 2.5 hours of HGF treatment as compared to six hours for Mig-7 (FIGS. 2A-2C). These experiments have been repeated several times with different lots of RL95 cells and different lots of recombinant HGF from different suppliers (R&D Systems and Sigma). Taken together, this data indicates that Mig-7 is consistently upregulated by SF treatment in the carcinoma cell lines, RL95 and HEC1A. In addition, these results show that the method of isolation of Mig-7 by SSH was validated.

### Example 12

### Mig-7 is Expressed in Metastatic Tumors

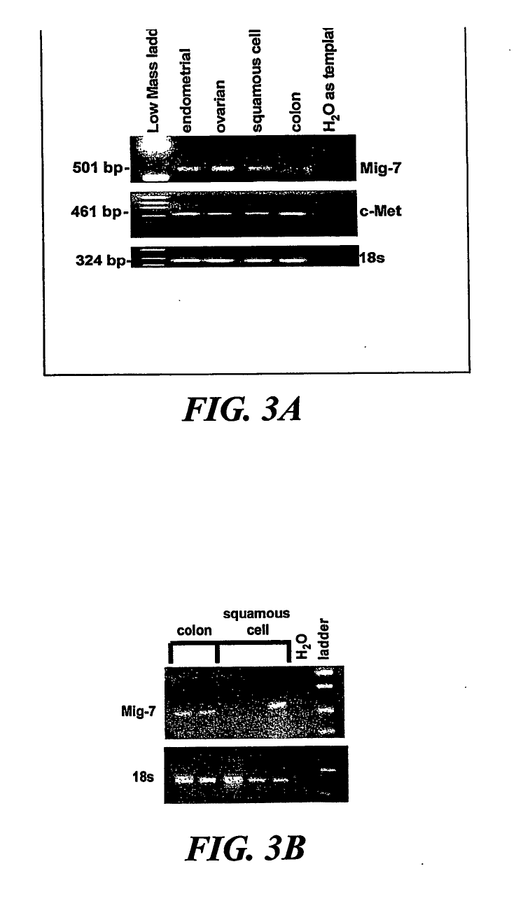

To investigate in vivo expression of Mig-7, RT-PCR was performed using Mig-7-, c-Met-, and 18s-specific primers. Mig-7 amplified products (expected size 501 bp) were detected in 100% of the metastatic tumors tested (endometrial, ovarian, lung squamous cell, and colon). In each case, the expected amplified c-Met product (450 bp) was also present (FIG. 3A). After examining colon and squamous cell metastatic tumors from additional individuals (FIG. 3B), it was demonstrated that all together, 100% of the colon carcinomas and 50% of the squamous cell carcinomas were positive for Mig-7 expression (FIGS. 3A & 3B). In this regard, it is interesting that HGF has been shown to cause the migration of, and invasion by, squamous carcinoma cells and that it enhances the adhesion of metastatic colon cancer cells to vascular endothelium (Fujisaki et al., “CD44 Stimulation Induces Integrin-Mediated Adhesion of Colon Cancer Cell Lines to Endothelial Cells by Up-Regulation of Integrins, c-Met and Activation of Integrins,” Cancer Research 59:4427-4434 (1999), which is hereby incorporated by reference in its entirety).

It is hypothesized that HGF may not be available to all of the cells, and, thus, Mig-7 expression may only be localized to the invasive edge of the tumor, as is Met and HGF expression (Vande Woude et al., “Met-HGF/SF: Tumorigenesis, Invasion and Metastasis,” Ciba Foundation Symposium 212:119-130 (1997); To et al., “The Roles of Hepatocyte Growth Factor/Scatter Factor and Met Receptor in Human Cancers,” Oncology Reports 5:1013-1024 (1998); Wagatsuma et al., “Tumor Angiogenesis, Hepatocyte Growth Factor, and c-Met Expression in Endometrial Carcinoma,” Cancer 82:520-530 (1998), which are hereby incorporated by reference in their entirety). The endometrial carcinoma expression is consistent with isolation from and expression of Mig-7 in the RL95 endometrial carcinoma cell line. The expression of Mig-7 in metastatic ovarian, oral squamous cell, and colon metastatic tumors is consistent with research showing a correlation of HGF and c-Met expression and invasiveness in these types of cancers (Di Renzo et al., “Overexpression of the Met/HGF Receptor in Ovarian Cancer,” International Journal of Cancer 58:658-662 (1994); Morello et al., “Met Receptor is Overexpressed but Not Mutated in Oral Squamous Cell Carcinomas,” Journal of Cellular Physiology 189:285-290 (2001); Fazekas et al., “Experimental and Clinicopathologic Studies on the Function of the HGF Receptor in Human Colon Cancer Metastasis,” Clinical & Experimental Metastasis 18:639-649 (2000), which are hereby incorporated by reference in their entirety). These results show that Mig-7 mRNA is not an artifact of immortalized cell lines since human metastatic tumors also express Mig-7 transcripts.

### Example 13

### Mig-7 mRNA is Specific to Carcinoma Cells and cannot be Induced in Primary Endometrial Epithelial Cells

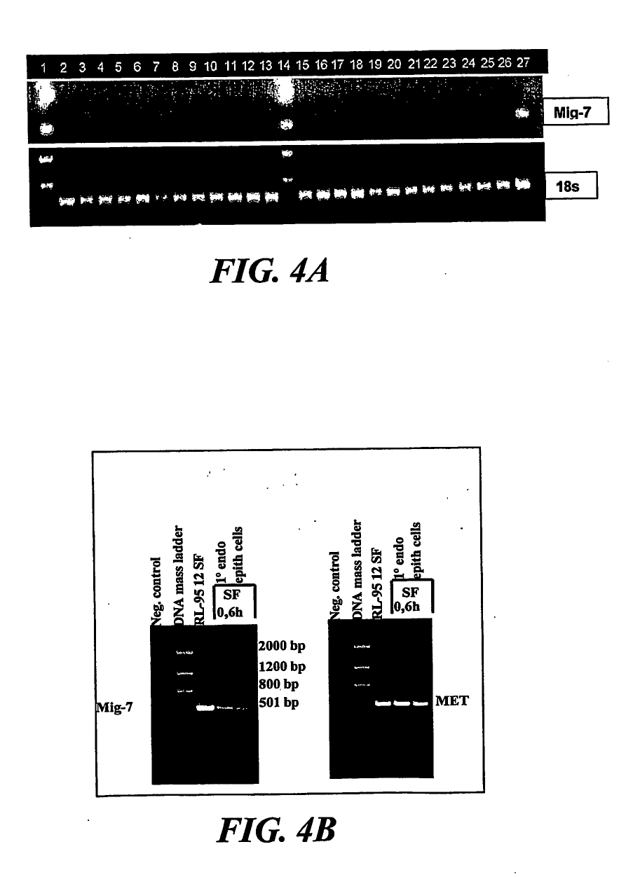

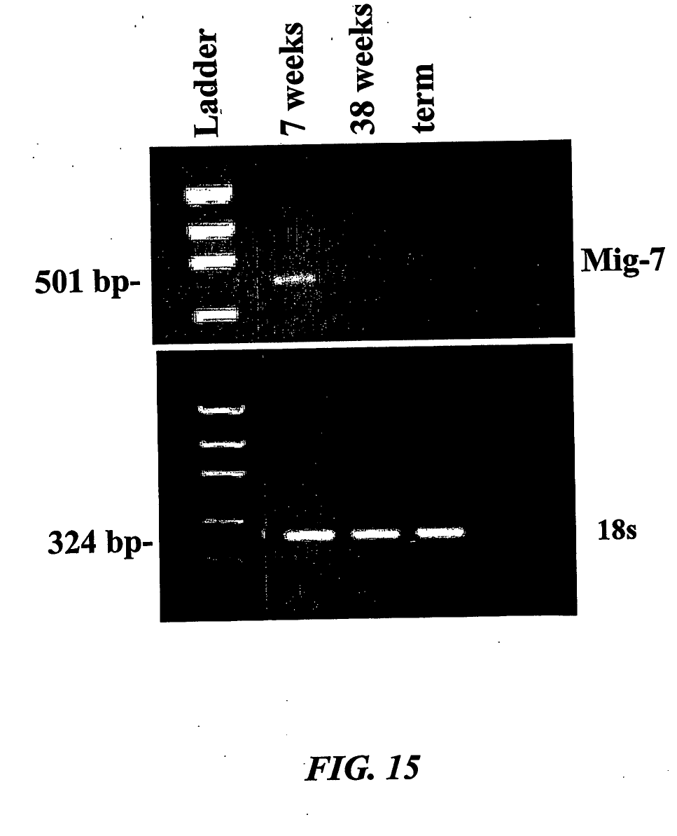

To test the hypothesis that Mig-7 expression is carcinoma cell specific, normal, non-cancerous human tissues were analyzed by poly A+ Northern blot and by RT-PCR. Mig-7 was not detectable by Northern blot of Poly A+ RNA from twelve different human tissues. As a result, the more sensitive method of RT-PCR was used to analyze Mig-7 expression in 24 different human tissues (FIG. 4A). Tissues, such as placenta, spleen, liver, small intestine, fetal liver, bone marrow, testis, ovary, and uterus, have been shown to express both HGF and c-Met (Fuller et al., “The Effect of Hepatocyte Growth Factor on the Behaviour of Osteoclasts,” Biochem. Biophys. Res. Commun. 212:334-340 (1995); Grano et al., “Hepatocyte Growth Factor is a Coupling Factor for Osteoclasts and Osteoblasts In Vitro,” Proceedings of the National Academy of Science 93:7644-7648 (1996); Sugawara et al., “Hepatocyte Growth Factor Stimulated Proliferation, Migration, and Lumen Formation of Human Endometrial Epithelial Cells In Vitro,” Biology of Reproduction 57:936-942 (1997); Uehara et al., “Placental Defect and Embryonic Lethality in Mice Lacking Hepatocyte Growth Factor/Scatter Factor,” Nature 373:702-705 (1995); Clark et al., “Hepatocyte Growth Factor/Scatter Factor and its Receptor c-met: Localisation and Expression in the Human Placenta Throughout Pregnancy,” Journal of Endocrinology 151:459467 (1996); Lail-Trecker et al., “A Role for Hepatocyte Growth Factor/Scatter Factor in Regulating Normal and Neoplastic Cells of Reproductive Tissues,” Journal of the Society of Gynecological Investigations 5:114-121 (1998); Lindsey et al., “Novel Hepatocyte Growth Factor/Scatter Factor Isoform Transcripts in the Macaque Endometrium and Placenta,” Molecular Human Reproduction 8:81-87 (2002); Matsumoto et al., “Hepatocyte Growth Factor (HGF) as a Tissue Organizer for Organogenesis and Regeneration,” Biochem Biophys Res Commun 239:639-644 (1997); Moriyama et al., “Up-Regulation of Vascular Endothelial Growth Factor Induced by Hepatocyte Growth Factor/Scatter Factor Stimulation in Human Glioma Cells,” Biochemistry and Biophysiology Research Communications 249:73-77 (1998); Parrott et al., “Developmental and Hormonal Regulation of Hepatocyte Growth Factor Expression and Action in the Bovine Ovarian Follicle,” Biology of Reproduction 59:553-560 (1998); Weimar et al., “Hepatocyte Growth Factor/Scatter Factor (HGF/SF) is Produced by Human Bone Marrow Stromal Cells and Promotes Proliferation, Adhesion and Survival of Human Hematopoietic Progenitor Cells (CD34+),” Experimental Hematology 26:885-894 (1998); Zachow et al., “Hepatocyte Growth Factor Regulates Ovarian Theca-Interstitial Cell Differentiation and Androgen Production,” Endocrinology 138:691-697 (1997), which are hereby incorporated by reference in their entirety), yet these tissues do not express detectable levels of Mig-7 transcripts even by RT-PCR. Since these pooled placenta samples in these assays were from term placentas, these results are consistent with first and second trimester placenta cytotrophoblasts cells being the most invasive in response to HGF as compared to third trimester placenta which is growth responsive to HGF (Dokras et al., “Regulation of Human Cytotrophoblast Morphogenesis Hepatocyte Growth Factor/Scatter Factor,” Biology of Reproduction 65:1278-1288 (2001), which is hereby incorporated by reference in its entirety). Mig-7 transcripts have been detected in early placental samples (prior to 22 weeks of gestation) (FIG. 15).

HGF has different effects on isolated primary endometrial epithelial cells (EEC) in vitro. Sugarwa et al. have shown that primary EEC undergo migration, tubule formation and mitosis in vitro (Sugawara et al., “Hepatocyte Growth Factor Stimulated Proliferation, Migration, and Lumen Formation of Human Endometrial Epithelial Cells In Vitro,” Biology of Reproduction 57:936-942 (1997), which is hereby incorporated by reference in its entirety). In contrast, Bae-Jump et al. have shown that carcinoma EEC (RL95 and HEC-1A) strictly migrate and invade under HGF stimulation (Bae-Jump et al., “Hepatocyte Growth Factor (HGF) Induces Invasion of Endometrial Carcinoma Cell Lines In Vitro,” Gynecologic Oncology 73:265-272 (1999), which is hereby incorporated by reference in its entirety). Taken together, these two studies imply that different genes are HGF-regulated in primary as compared to carcinoma EEC. To further test the hypothesis that Mig-7 expression is carcinoma cell- and migration-specific, primary EEC was isolated and cultured with or without HGF and assayed by relative RT-PCR for Mig-7 and c-Met expression. Mig-7 is induced in the control RL95 cells but not in primary EEC isolated from normal human endometrium (FIG. 4B). Primary EEC express c-Met mRNA (FIG. 4B) suggesting that they are capable of responding to HGF. These results are consistent with Mig-7 expression being carcinoma cell specific and with HGF causing different effects in primary as compared to carcinoma EEC.

### Example 14

### Blocking Antibody to αvβ5 Inhibits HGF Induction of Mig-7 Expression

It was hypothesized that integrin expression and activation play an important role in Mig-7 expression. This hypothesis is based on the fact that HGF has been shown to cause the migration of and invasion of squamous carcinoma cells and to activate focal adhesion kinase (FAK), a protein involved in integrin signaling (Beviglia et al., “HGF Induces FAK Activation and Integrin-Mediated Adhesion in MTLn3 Breast Carcinoma Cells,” International Journal of Cancer 83:640-649 (1999); Trusolino et al., “Growth Factor-Dependent Activation of avb3 Integrin in Normal Epithelial Cells: Implications for Tumor Invasion,” Journal of Cell Biology 142:1145-1156 (1998); Matsumoto et al., “Hepatocyte Growth Factor/Scatter Factor Induces Tyrosine Phosphorylation of Focal Adhesion Kinase (p125^{FAK}) and Promotes Migration and Invasion by Oral Squamous Cell Carcinoma Cells,” Journal of Biological Chemistry 269:31807-31820 (1994), which are hereby incorporated by reference in their entirety). Furthermore, it has been shown that HGF does not induce Mig-7 expression in normal endometrial epithelial cells (FIG. 4B) and previous work shows that that primary EEC respond differently than do carcinoma EEC (Bae-Jump et al., “Hepatocyte Growth Factor (HGF) Induces Invasion of Endometrial Carcinoma Cell Lines In Vitro,” Gynecologic Oncology 73:265-272 (1999); Sugawara et al., “Hepatocyte Growth Factor Stimulated Proliferation, Migration, and Lumen Formation of Human Endometrial Epithelial Cells In Vitro,” Biology of Reproduction 57:936-942 (1997), which are hereby incorporated by reference in their entirety). These data taken together with previous reports that the pleiotropic effects of HGF and integrins are dependent upon and regulated by the differentiation state of the cell (Birchmeier et al., “Role of HGF/SF and c-Met in Morphogenesis and Metastasis of Epithelial Cells,” Ciba Foundation Symposium 212:230-246 (1997); Fehlner-Gardiner et al., “Characterization of a Functional Relationship Between Hepatocyte Growth Factor and Mouse Bone Marrow-Derived Mast Cells,” Differentiation 65:27-42 (1999); Byers et al., “Breast Carcinoma: A Collective Disorder,” Breast Cancer Research & Treatment 31:203-215 (1994), which are hereby incorporated by reference in their entirety) form the basis of testing integrin signaling with respect to Mig-7 expression.

The effects of integrin blocking antibodies specific to integrins involved in cell migration, including β1 subunit, αvβ5, and αvβ6, have been tested. Integrins with β1 subunit have been shown to be important for adhesion and motility of MTLn3 breast carcinoma cells (Beviglia et al., “HGF Induces FAK Activation and Integrin-Mediated Adhesion in MTLn3 Breast Carcinoma Cells,” International Journal of Cancer 83:640-649 (1999), which is hereby incorporated by reference in its entirety). In addition, cohort migration of melanoma cells relies on β1 integrin function (Hegerfeldt et al., “Collective Cell Movement in Primary Melanoma Explants: Plasticity of Cell-Cell Interaction, b1 Integrin Function, and Migration Strategies,” Cancer Research 62:2125-2130 (2002), which is hereby incorporated by reference in its entirety). αvβ5 has been implicated in glioma and squamous cell carcinoma invasion in vivo (Jones et al., “Changes in the Expression of Alpha v Integrins in Oral Squamous Cell Carcinomas,” Journal of Oral Pathology & Medicine 26:63-68 (1997), which is hereby incorporated by reference in its entirety) and to be required for tyrosine kinase receptor induced invasion in pancreatic carcinoma, FG, cells in vitro (Klemke et al., “Receptor Tyrosine Kinase Signaling Required for Integrin Alpha v Beta 5-Directed Cell Motility but Not Adhesion on Vitronectin,” Journal of Cell Biology 127:859-866 (1994), which is hereby incorporated by reference in its entirety). The αvβ6 integrin has been detected and shown to be activated in several types of carcinoma cells (Breuss et al., “Expression of the b6 Integrin Subunit in Development, Neoplasia and Tissue Repair Suggests a Role in Epithelial Remodeling,” Journal of Cell Science 108:2241-2251 (1995), which is hereby incorporated by reference in its entirety). All three integrin blocking antibodies, β1 antibody (GS6), αvβ5 antibody (P1F6), and αvβ6 antibody (10D5) have been shown to block binding and activation of their respective integrins (Munger et al., “The Integrin Alpha v Beta 6 Binds and Activates Latent TGF Beta 1: A Mechanism for Regulating Pulmonary Inflammation and Firosis,” Cell 96:319-328 (1999); Gao et al., “Migration of Human Polymorphonuclear Leukocytes Through a Synovial Fibroblast Barrier is Mediated by Both Beta 2 (CD11/CD18) Integrins and the Beta 1 (CD29) Integrins VLA-5 and VLA-6,” Cellular Immunology 163:178-186 (1995); Wayner et al., “Integrins Alpha v Beta 3 and Alpha v Beta 5 Contribute to Cell Attachment to Vitronectin but Differentially Distribute on the Cell Surface,” Journal of Cell Biology 113:919-929 (1991), which are hereby incorporated by reference in their entirety).

Most studies using integrin blocking antibodies remove the cells from the tissue culture plate, treat the cells with integrin blocking antibody and then replate the cells to determine adhesion and migration. Because cancer cells can lay down and modify ECM differently than do normal cells (Emoto et al., “Annexin II Overexpression Correlates with Stromal Tenascin-C Overexpression: A Prognostic Marker in Colorectal Carcinoma,” Cancer 92:1419-1426 (2001); Euer et al., “Identification of Genes Associated with Metastasis of Mammary Carcinoma in Metastatic Versus Non-Metastatic Cell Lines,” Anticancer Research 22:733-740 (2002); Matsuyama et al., “Comparison of Matrix Metalloproteinase Expression Between Primary Tumors With or Without Liver Metastasis in Pancreatic and Colorectal Carcinomas,” Journal of Surgical Oncology 80:105-110 (2002), which are hereby incorporated by reference in their entirety), the cells were plated for four days in the absence of exogenous extracellular matrix (ECM) to allow time for ECM deposition and modification. The cells were cultured under the same conditions used to isolate Mig-7.

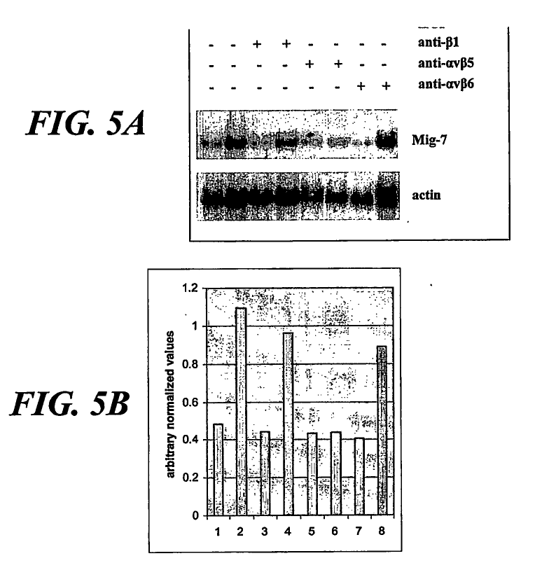

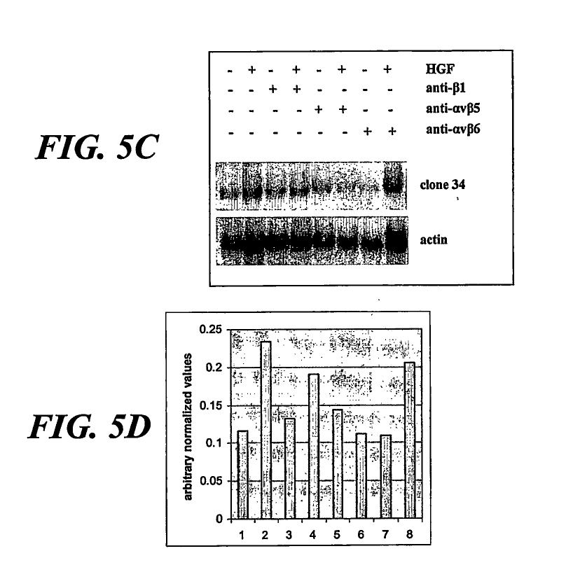

FIGS. 5A and 5B demonstrate that blocking antibody to αvβ5 (P1F6) prevented the normal six-hour HGF induction of Mig-7. This antibody also blocked expression of another clone, 34 (FIGS. 5C and 5D), that was isolated at the same time as Mig-7. In contrast to αvβ5 blocking antibody, treatment with blocking antibodies to β1 or to αvβ6 integrins did not prevent HGF induction of Mig-7 (FIGS. 5A and 5B) even though both αvβ6 and αvβ5 integrins bind to vitronectin (Huang et al., “The Integrin αvβ5 is Critical for Keratinocyte Migration of Both its Known Ligand, Fibronectin, and on Vitronectin,” Journal of Cell Science 111:2189-2195 (1998), which is hereby incorporated by reference in its entirety). Whereas, β1 blocking antibody had a less than 2 fold reduction of clone 34 expression (FIGS. 5C and 5D). This experiment has been repeated and results show the same inhibition of Mig-7 induction using blocking antibody (P1F6) to αvβ5. These data suggest that Mig-7 expression requires αvβ5 binding. Whether or not this binding is to a known or unknown αvβ5 ligand remains to be determined.

Accumulating evidence suggests that αvβ5 interacts with the HGF/Met/Mig-7 pathway. αvβ5 integrins were found on 17 oral squamous cell carcinomas (Jones et al., “Changes in the Expression of Alpha v Integrins in Oral Squamous Cell Carcinomas,” Journal of Oral Pathology & Medicine 26:63-68 (1997), which is hereby incorporated by reference in its entirety), which is the same type of metastatic cancer that was found expressed Mig-7 (FIGS. 3A,3B). Blocking antibodies to αvβ5 inhibited invasion of human gliomas into rat brain aggregates (Tonn et al., “Invasive Behaviour of Human Gliomas is Mediated by Interindividually Different Integrin Patterns,” Anticancer Research 18:2599-2605 (1998), which is hereby incorporated by reference in its entirety). More importantly, αvβ5 has been reported to play a role in tyrosine kinase receptor activation-dependent cell migration (Klemke et al., “Receptor Tyrosine Kinase Signaling Required for Integrin Alpha v Beta 5-Directed Cell Motility but Not Adhesion on Vitronectin,” Journal of Cell Biology 127:859-866 (1994), which is hereby incorporated by reference in its entirety). In CS-1 melanoma, MCF-7PB breast carcinoma, and FG pancreatic carcinoma cells that express αvβ5 but not αvβ3, binding of both insulin-like growth factor receptor and αvβ5 is required for spontaneous pulmonary metastasis but is not required for primary tumor growth (Brooks et al., “Insulin-Like Growth Factor Receptor Cooperates with Integrin Alpha v Beta 5 to Promote Tumor Cell Dissemination In Vivo,” Journal of Clinical Investigation 99:1390-1398 (1997), which is hereby incorporated by reference in its entirety).

The observation that HGF does not induce Mig-7 expression in primary endometrial epithelial cells even though these cells express the HGF receptor (FIG. 4B), suggests that the Met signaling pathway bifurcates. In carcinoma cells, the Mig-7 induction pathway is activated, while in normal cells, this pathway is suppressed. It is thus likely that cross-talk between the HGF/Met pathway and integrin signal transduction pathways determine whether or not Mig-7 induction occurs. Integrin expression and signal transduction pathways regulate the state of cell differentiation (Bokel et al., “Integrins in Development: Moving On, Responding To, and Sticking to the Extracellular Matrix,” Developmental Cell 3:311-321 (2002); van der Flier et al., “Function and Interactions of Integrins,” Cell & Tissue Research 305:285-298 (2001), which are hereby incorporated by reference in their entirety). Since carcinoma cells are typically in a less differentiated state than normal cells, it is not surprising that marked differences exist in integrin expression and signaling between normal and cancer cells (Giancotti et al., “Integrin Signaling,” Science 285:1028-1032 (1999), which is hereby incorporated by reference in its entirety). These results suggest specific cross talk exists between HGF/Met and αvβ5 signal transduction pathways. Collectively, these data suggest that αvβ5 integrin expression is required for HGF-induced Mig-7 expression that may explain why primary endometrial epithelial cells cannot be induced by HGF to express Mig-7. Previous work shows a lack of αvβ5 expression in normal endometrial epithelial cells (Lessey et al., “Luminal and Glandular Endometrial Epithelium Express Integrins Differentially Throughout the Menstrual Cycle: Implications for Implantation, Contraception, and Infertility,” American Journal of Reproductive Immunology 35:195-204 (1996), which is hereby incorporated by reference in its entirety). However, migration of cytotrophoblasts in early placenta require αvβ5 for migration and invasion of the maternal blood supply (Zhou et al., “Human Cytotrophoblasts Adopt a Vascular Phenotype As They Differentiate,” Journal of Clinical Investigation 99(9):2139-2151 (1997), which is hereby incorporated by reference in its entirety). These results suggest that Mig-7 expression is a result of both cytokine and adhesion receptor ligation. Studies to further elucidate the specific point of convergence of these signaling pathways are currently being conducted.

### Example 15

### Mig-7 Antisense Inhibits Carcinoma Cell Migration In Vitro

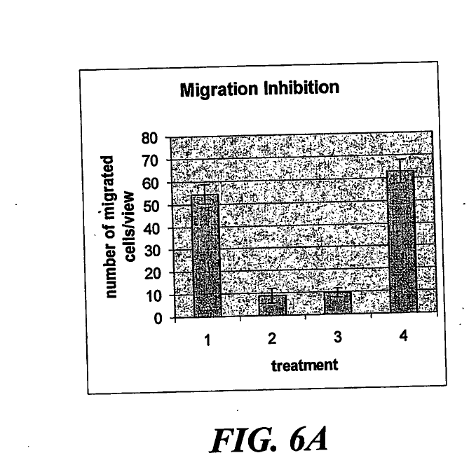

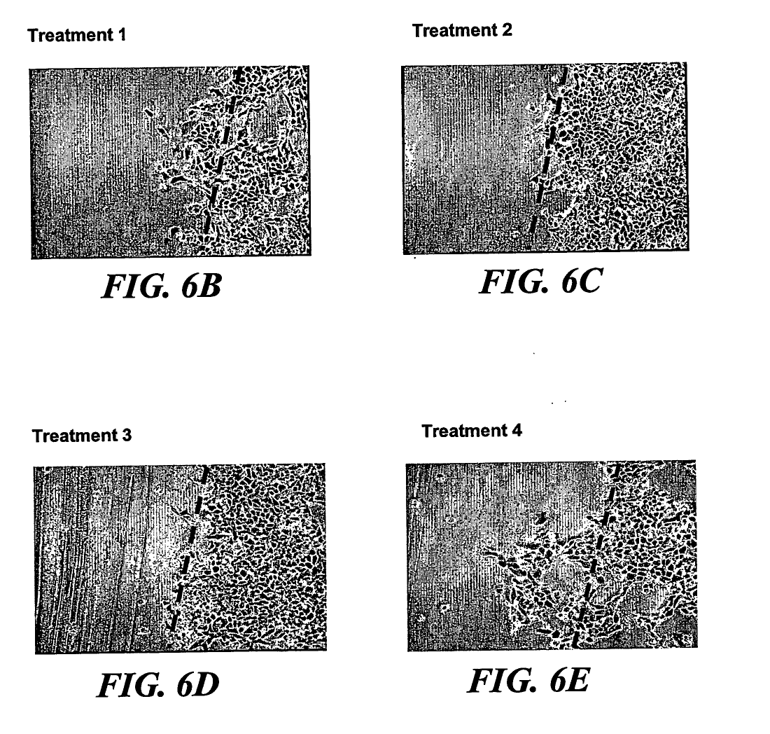

A time course of RL95 cells showed that migration of these cells does not commence until after six hours of HGF treatment (FIGS. 2A-2C). Since Mig-7 is expressed prior to that time (FIGS. 2D, 2E), its expression may regulate migration. To test that hypothesis, antisense oligonucleotides (ODNs) specific to the Mig-7 were used to treat RL95 cells that had been stimulated to migrate with HGF. Migration after 24 hours was documented and quantified. Treatment of RL95 cells with the antisense ODNs, treatment 2 and 3, surrounding the Mig-7 Kozak consensus sequence (FIG. 1A) and 5′ of that region inhibited HGF-induced migration by 83.50±2.77% and 82.21±3.18%, respectively (p<0.05) when compared to treatment with an irrelevant ODN comprised of the inverted sequence to the 5′ Mig-7 ODN (FIG. 6A). Images of the resultant migration for each treatment can been seen in FIGS. 6B-6E. This is the first evidence demonstrating that the use of antisense ODNs to an HGF-induced transcript can inhibit carcinoma cell migration in vitro.

HGF causes an epithelial to mesenchyme transition in cellular morphology (Birchmeier et al., “Role of HGF/SF and c-Met in Morphogenesis and Metastasis of Epithelial Cells,” Ciba Foundation Symposium 212:230-246 (1997); Fournier et al., “Cbl-Transforming Variants Trigger a Cascade of Molecular Alterations that Lead to Epithelial Mesenchymal Conversion,” Molecular Biology of the Cell 11:3397-3410 (2000); Boyer et al., “Induction and Regulation of Epithelial-Mesenchymal Transitions,” Biochemical Pharmacology 60:1091-1099 (2000), which are hereby incorporated by reference in their entirety). This transition along with HGF-induced migration is associated with a resistance to apoptosis (Matsumoto et al., “Hepatocyte Growth Factor (HGF) as a Tissue Organizer for Organogenesis and Regeneration,” Biochem Biophys Res Commun 239:639-644 (1997), which is hereby incorporated by reference in its entirety). Mig-7 may be involved in this anti-apoptotic pathway as well since migration is coordinately regulated with survival through activation and molecular coupling of p130 Crk-associated substrate (CAS) and c-CrkII (Cho et al., “Extracellular-Regulated Kinase Activation and CAS/Crk Coupling Regulate Cell Migration and Suppress Apoptosis During Invasion of the Extracellular Matrix,” Journal of Cell Biology 149:223-236 (2000), which is hereby incorporated by reference in its entirety) and CrkII expression is required for HGF-mediated E-cadherin breakdown (Lamorte et al., “Crk Adapter Proteins Promote an Epithelial-Mesenchymal-Like Transition and are Required for HGF-Mediated Cell Spreading and Breakdown of Epithelial Adherens Junctions,” Molecular Biology of the Cell 13:1449-1461 (2002), which is hereby incorporated by reference in its entirety) prior to migration. In addition, HGF has been shown to inhibit apoptosis through the activation of c-Met and increased expression of the anti-apoptotic protein bcl-w (Kitamura et al., “Met/HGF Receptor Modulates bcl-w Expression and Inhibits Apoptosis in Human Colorectal Cancers,” British Journal of Cancer 83:668-673 (2000), which is hereby incorporated by reference in its entirety). Whether or not Mig-7 plays a role in this anti-apoptotic state during migration is to be determined.

In conclusion, it has been demonstrated for the first time that HGF treatment and binding of αvβ5 integrin induces a novel, cancer-cell specific transcript that is required for carcinoma cell migration. Because expression of HGF and activation of its receptor c-Met leads to invasion and metastasis of cancer cells (Vande Woude et al., “Met-HGF/SF: Tumorigenesis, Invasion and Metastasis,” Ciba Foundation Symposium 212:119-130 (1997); To et al., “The Roles of Hepatocyte Growth Factor/Scatter Factor and Met Receptor in Human Cancers,” Oncology Reports 5:1013-1024 (1998); Tamagnone et al., “Control of Invasive Growth by Hepatocyte Growth Factor and Related Scatter Factors,” Cytokine and Growth Factor Reviews 8:129-142 (1997), which are hereby incorporated by reference in their entirety), αvβ5 integrin cooperates with tyrosine kinase receptor activated invasion and metastasis (Klemke et al., “Receptor Tyrosine Kinase Signaling Required for Integrin Alpha v Beta 5-Directed Cell Motility but Not Adhesion on Vitronectin,” Journal of Cell Biology 127:859-866 (1994), which is hereby incorporated by reference in its entirety) and Mig-7 is induced by HGF with αvβ5 signaling, Mig-7 expression may play a role in metastasis in vivo. Because Mig-7 specific antisense olignucleotides inhibit RL95 carcinoma cell migration in vitro, Mig-7 may be a cancer cell-specific target in vivo.

### Example 16

### RL95 Cell Invasiveness In Vivo

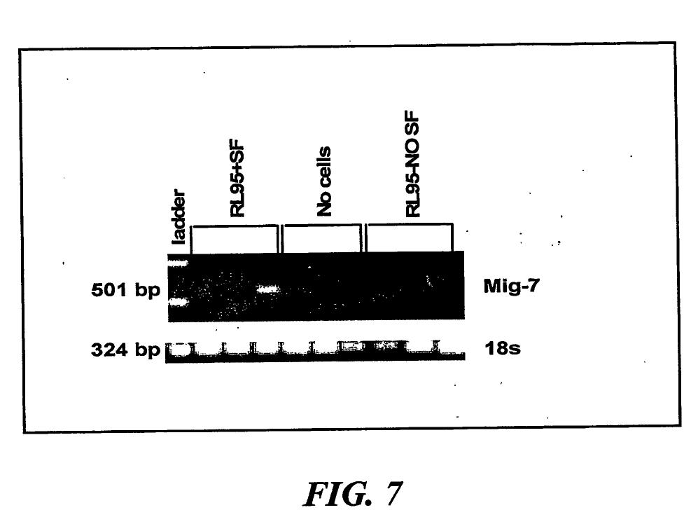

A nude mouse model has been used to examine the invasiveness of Mig-7 expressing RL95 cells. First, RL95 cells were treated with SF as previously described. Then, 1×10⁵ SF-treated cells were combined with Matrigel™ low growth factor reagent (500 μl) and injected it subcutaneously into nude mice under IACUC approval. Negative controls included Matrigel alone (i.e. no cells), or Matrigel plus serum starved cells not treated with SF. Primary tumors were allowed to reach one cm³ before the mice were euthanized for blood and tissue collection. By RT-PCR, Mig-7 was detected in the blood from a nude mouse injected with SF treated RL95 cells but not from a mouse injected with Matrigel alone (FIG. 7); Mig-7 was also not detected in the Matrigel plus serum starved cells not treated with SF (FIG. 7). Other tissue samples from these mice are being tested. There are plans to also test the invasiveness of RL95 cells and other Mig-7 expressing cell lines (HEC1A and FG) after orthotopic injections since the delivery route may have an effect on the invasiveness of the respective cell line (Killion et al., “Orthotopic models are necessary to predict therapy of transplantable tumors in mice,” Cancer Metastasis Review 17:279-284 (1999), which is incorporated by reference in its entirety). Although these are single mouse results, this preliminary data is very exciting and suggests that RL95 cells can invade the vascular system.

### Example 17

### Isolation of MIG-7 cDNA

Using a highly sensitive PCR-based method of suppression subtraction hybridization (SSH), Mig-7 cDNA has been isolated. The method used, suppressive subtraction hybridization (Diatchenko et al., “Suppression Subtractive Hybridization: A Method for Generating Differentially Regulated or Tissue-Specific cDNA Probes and Libraries,” Proceedings of the National Academy of Science 93:6025-6030 (1996), which is hereby incorporated by reference in its entirety), is particularly useful in isolation of low-abundance sequences; a characteristic typical of transiently expressed, tissue-specific mRNAs. Genes induced rather than downregulated by SF were specifically targeted for isolation. The six-hour SF induction period was focused on because migration of RL-95 cells starts at 12 hours and many cell-specific genes, such as transcription factors, are expressed during that time. The RL-95 human endometrial epithelial carcinoma cell line derived from a Grade 2 moderately differentiated adenosquamous carcinoma of the endometrium (Way et al., “Characterization of a New Human Endometrial Carcinoma (RL95-2) Established in Tissue Culture,” In Vitro 19:147-158 (1983), which is hereby incorporated by reference in its entirety) was used This cell line was chosen based on the expression of c-Met but not SF as verified by literature searches, immunohistochemistry for c-Met, and RNase protection analysis for SF transcripts (Moghul et al., “Modulation of c-MET Proto-Oncogene (HGF Receptor) mRNA Abundance by Cytokines and Hormones: Evidence for Rapid Decay of the 8 kb c-MET Transcript,” Oncogene 9:2045-2052 (1994), which is hereby incorporated by reference in its entirety). It was also chosen because it is a carcinoma cell line and migrates with SF treatment (Bae-Jump et al., “Hepatocyte Growth Factor (HGF) Induces Invasion of Endometrial Carcinoma Cell Lines In Vitro,” Gynecologic Oncology 73:265-272 (1999), which is hereby incorporated by reference in its entirety). In addition, because SF and c-Met hormonal regulation and localization in normal endometrium has been studied, experience with the normal histology of this tissue and isolating normal endometrial epithelium in order to evaluate the cancer cell specificity of Mig-7 was available.

Because this cell line expresses the estrogen receptor (ER) (Way et al., “Characterization of a New Human Endometrial Carcinoma (RL95-2) Established in Tissue Culture,” In Vitro 19:147-158 (1983), which is hereby incorporated by reference in its entirety) and phenol red has been shown to affect this receptor, cells were cultured in DMEM/F12 media without serum or phenol red for two days before SF treatment (40 ng/mL media). Cells were at approximately 70% confluency. No additional extracellular matrix was added to the plates before plating the cells. Since SSH only isolates segments of cDNAs, rapid amplification of cDNA ends (RACE) was performed using Mig-7 specific nested 3′ reverse primers and RLM-RACE™ (Ambion) to isolate the 5′ end. The 3′ end had been originally isolated based on the polyadenylation signal sequence (underlined in FIG. 1A) and a string of adenosines (not shown) in the SSH isolated fragment. After successful amplification, the PCR products were cloned into the pCRII vector (Invitrogen, Carlsbad, Calif.), screened for inserts, and sequenced. Eight clones of Mig-7 were sequenced in both directions and a concensus sequence was determined by analysis with the Vector NTI™ program (FIG. 1).

Mig-7 homologies of 99% identity were found with four human ESTs. Three of these ESTs were isolated at the National Cancer Institute in the Cancer Genome Anatomy Project (alignment data not shown due to the page limitation of this application). The fourth was from the Washington University-Merck EST Project. Only one EST (accession number N41315) was homologous to the 5′ region of Mig-7. Two of the ESTs (N41315 and AI18969) were isolated from early (weeks 8 to 9) placenta. Whereas the other two (ESTs BE644624 and AA971972) were isolated from pooled libraries from carcinomas. Protein homology searches using translations in all six reading frames (forward and reverse) show no significant homology to any database sequence. There is a repeat of thymidines and guanosines which should encode a cystein-rich region. Also, no full-length cDNA homologies were found in the databases. In the SAGE database, 3′ Mig-7 homology was found in the ovarian carcinoma cell line OV1063 library but not in the twelve normal cell SAGE libraries. Therefore, these results suggest that a novel, SF-induced gene has been isolated.

### Example 18

### SF Induces Mig-7 in Multiple Cancer Cell Types

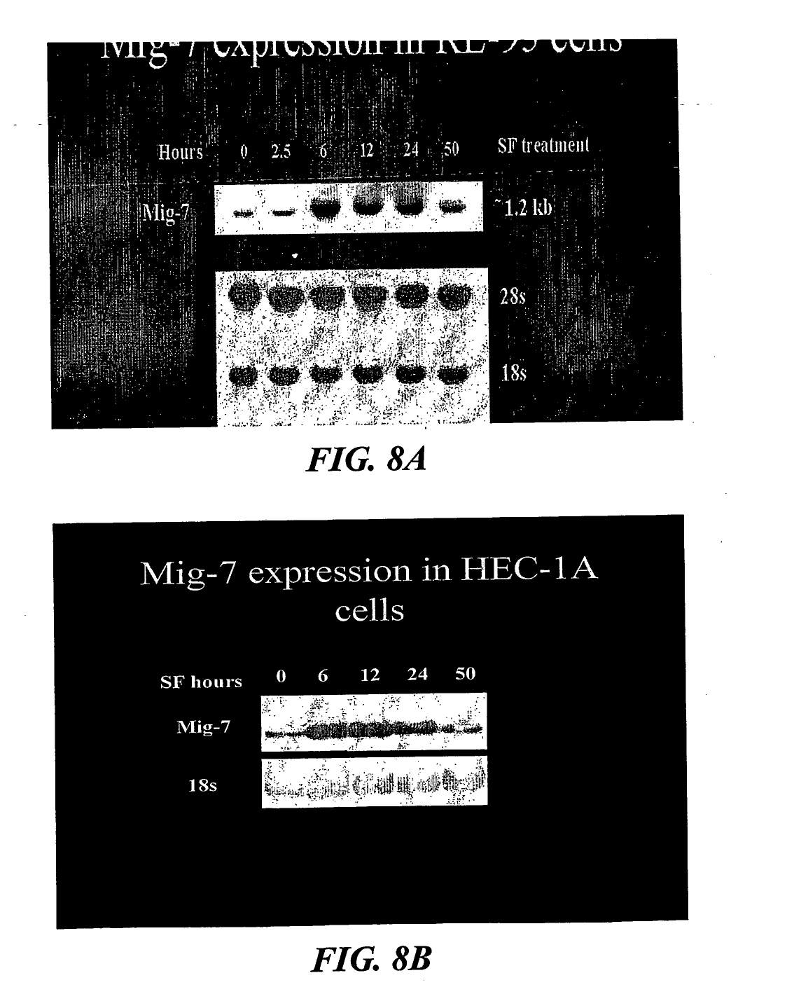

Northern blot analyses show that SF dramatically and reproducibly induced Mig-7 in RL-95 and HEC1A cells (both obtained from ATCC). The same culture conditions were used as for isolation of Mig-7 as described above. Over the time course of SF treatment of these cells, transcripts for this novel gene were highest at 6 hours of treatment (FIGS. 8A and 8B). The Nothern blots were stained with methylene blue to detect ribosomal RNA and show equal levels of RNA loaded in each lane. This experiment has been repeated at least three times with different lots of recombinant SF from different suppliers (Genentech and Sigma). Therefore, Mig-7 is upregulated by SF treatment in the endometrial carcinoma cell lines, RL-95 and HEC-1A.

### Example 19

### Mig-7 Expression in Normal Isolated Endometrial Cells In Vitro

Normal endometrial epithelial cells migrate to regenerate the functionalis region in vivo on days 3-5 of the cycle (Ferenczy et al., “Studies on the Cytodynamics of Human Endometrial Regeneration. III. In Vitro Short-Term Incubation Historadioautography,” American Journal of Obstetrics and Gynecology 134:297-304 (1979), which is hereby incorporated by reference in its entirety). This part of the cycle was reproduced in vitro by isolating primary human endometrial epithelial cells from three different hysterectomy patients with normal uteri (IRB approval 1678), pooling the cells and culturing them with or without SF. A previously documented cell isolation technique by Classen-Linke et al (Classen-Linke et al., “Establishment of a Human Endometrial Cell Culture System and Characterization of its Polarized Hormone Responsive Epithelial Cells,” Cell Tissue Research 287:171-185 (1997), which is hereby incorporated by reference in its entirety) was used. Epithelial cells were obtained as defined by their cuboidal morphology as opposed to the fibroblast morphology of stromal cells. As shown in FIG. 4B, Mig-7 was not induced by SF over basal levels as determined by RT-PCR analysis even after 40 cycles as compared to RL-95 carcinoma cells Mig-7 expression. This resulting amplified DNA was confirmed to be Mig-7 specific by transferring the amplified DNA to a membrane and performing Southern analysis using the random primed ³²P-labeled Mig-7 cDNA as a template.

PCR of SF receptor, c-Met, after 35 cycles shows relative equal expression between the carcinoma cells and the primary epithelial cells. Suggesting that the lack of induction is not due to a lack of SF receptor. The lack of Mig-7 induction in the primary endometrial epithelial cells does point to a possibility of “cross-talk” between the signaling of Met and other signaling pathways. One possibility is the signal transduction caused by integrin activation (Giancotti et al., “Integrin Signaling,” Science 285:1028-1032 (1999), which is hereby incorporated by reference in its entirety). Nevertheless, SF induces Mig-7 expression in RL-95 endometrial epithelial carcinoma cells but not in primary endometrial epithelial cells, suggesting that this expression is carcinoma cell specific.

### Example 20

### Mig-7 Expression is not Detected in Normal Tissue

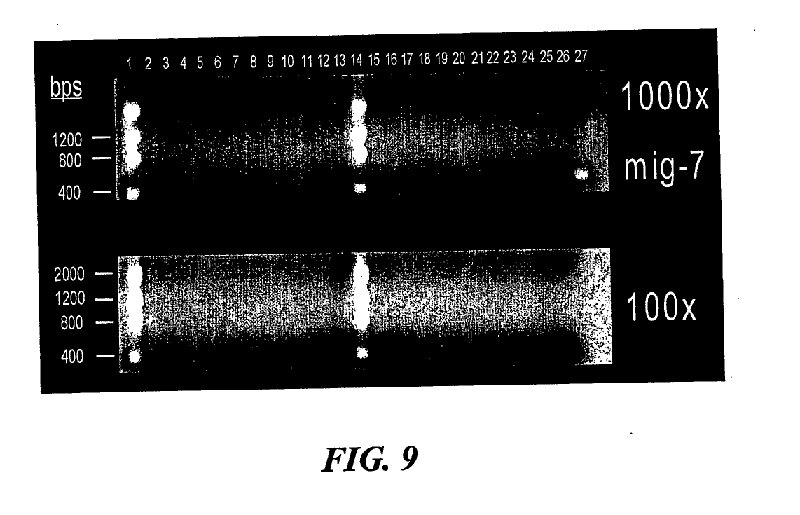

The expression of Mig-7 in normal human tissues has been examined both by Northern blot of Poly A+ RNA which is sensitive enough to detect lowly expressed genes and by RT-PCR (FIG. 9). The RT-PCR of the 24 human tissues (Rapid-Scan, OriGene) was repeated twice including a positive control (cDNA from 24 hour SF treated RL-95 cells). Both the blot and the Rapid-Scan contained pooled samples from several individuals and normalized to actin RNA or cDNA levels, respectively. The Rapid-Scan came in a 96-well format containing four different concentrations of the same cDNAs (1000×, 100×, 10×, 1×), with 1×at 1 pg per cDNA sample. These cDNAs were tested by OriGene for the presence of the lowly expressed genes transferrin receptor and ataxia telangiectasia Mig-7 expression was not detectably expressed in placenta, prostate, muscle, spleen, uterus, liver, lung, maxillary gland (FIG. 9) organs that have been shown to express SF/Met. Mig-7 expression was not detected in any normal adult tissue nor in fetal lung or fetal brain. Based on the fact that the EST homologies were from early placenta cDNA libraries previously mentioned, it was checked and determined that these were from term, not early, placentas. The expected size (501 bp) was not detected for the Mig-7 positive control (RL-95 SF-treated cells RNA). These data suggest that Mig-7 expression is carcinoma cell-specific.

### Example 21

### Method to Detect Cancer Cells

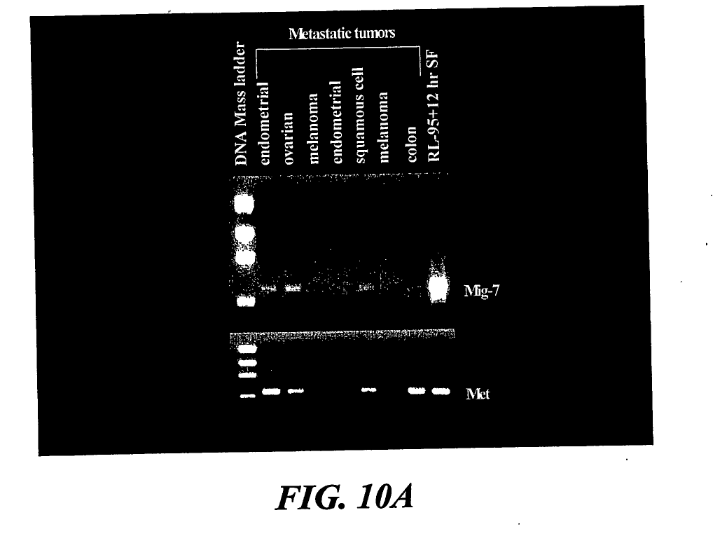

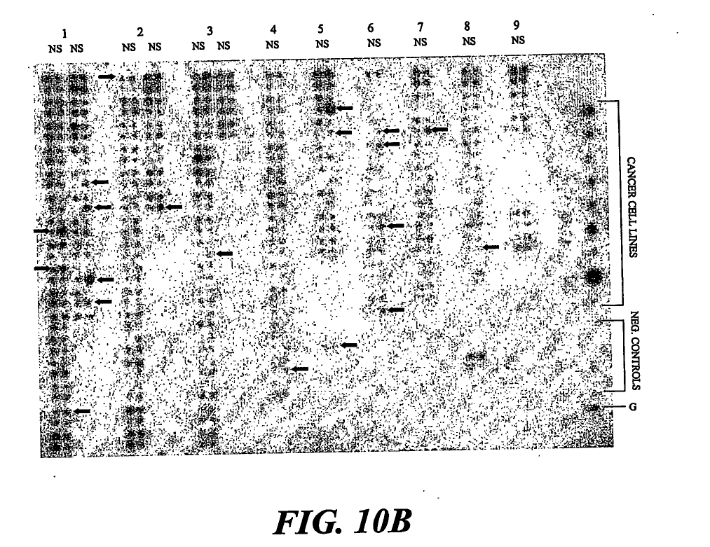

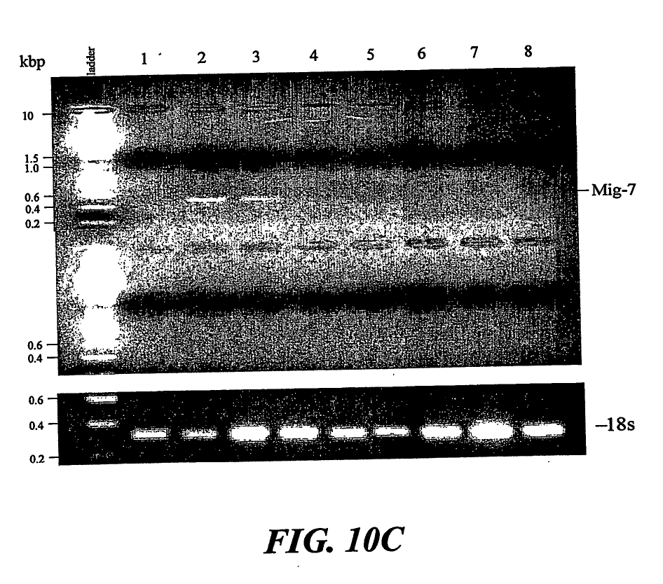

It has been hypothesized that Mig-7 was expressed in metastatic cancer tumors. In cooperation with the Cooperative Human Tissue Network (IRB approval 1678), tissue samples of tumors of metastatic cancers and isolated total RNA were obtained. The integrity of this RNA was determined by electrophoresis in an ethidium bromide stained, formaldehyde, 1% agarose gel. All RNA samples were intact. By RT-PCR using Mig-7 specific primers, human endometrial, ovarian, squamous cell and colon metastatic tumors were shown to express Mig-7 mRNA (FIGS. 10A-10C). It is interesting to note that SF has been shown to cause the migration of and invasion by squamous carcinoma cells and to activate FAK (Matsumoto et al., “Hepatocyte Growth Factor/Scatter Factor Induces Tyrosine Phosphorylation of Focal Adhesion Kinase (p125^{FAK}) and Promotes Migration and Invasion by Oral Squamous Cell Carcinoma Cells,” Journal of Biological Chemistry 269:31807-31820 (1994), which is hereby incorporated by reference in its entirety) a protein in the integrin signaling pathway. SF also enhances the adhesion of colon cancer cells to vascular endothelium (Fujisali et al., “CD44 Stimulation Induces Integrin-Mediated Adhesion of Colon Cancer Cell Lines to Endothelial Cells by Up-Regulation of Integrins, c-Met and Activation of Integrins,” Cancer Research 59:4427-4434 (1999), which is hereby incorporated by reference in its entirety).

These data imply that Mig-7 may be expressed by a very small population of tumor cells such as the ones on the outer periphery of the tumor that would be exposed to SF secreted by adjacent stromal cells. This is consistent with SF and c-Met expression localized to the invading edge of tumors (Vande Woude et al., “Met-HGF/SF: Tumorigenesis, Invasion and Metastasis,” Ciba Foundation Symposium 212:119-130 (1997), which is hereby incorporated by reference in its entirety). This data shows that detectable Mig-7 mRNA expression corresponds to Met mRNA expression in these samples (FIGS. 10A-10C) which supports the idea that Mig-7 mRNA is a result of Met activation. Consequently, preliminary evidence shows that Mig-7 is expressed in human metastatic cancer tissue and is not just specific to endometrial carcinoma cells. Other methods include standard detection methods of in situ hybridization and immunohistochemistry using Mig-7 specific probes and antibodies.

### Example 22

### Method of Detecting Migrating Cancer Cells in Normal Tissue

Using a cancer profiling array from Clontech, Mig-7 mRNA has been detected in tumors and in tissue surrounding tumors from 241 individual patients even though these tissues surrounding the tumor were deemed “normal” by pathologists' evaluations. Mig-7 mRNA was not detected in the negative controls but was highly expressed in the cancer cell lines. Highest Mig-7 expression was seen in cancer cell lines colorectal adenocarcinoma, SW480, lung carcinoma, A549, and cervical Hela (FIG. 10B). As can be seen in FIG. 10B marked with arrows, many samples were more positive for Mig-7 in the tumor than in surrounding tissue (Table 1) indicating that more cells in the tumor were preparing to migrate. While in other samples, the surrounding tissue displayed more Mig-7 expression (Table 2), suggesting that most of the tumor cells stimulated to migrate had already migrated into the surrounding tissue. However, in almost all patient samples the tissue surrounding the tumor was positive for Mig-7 expression (FIG. 10B) indicating that migrating cancer cells were present. This array is normalized so that each sample contains the same amount of cDNA made from the RNA for each sample. When probed with a housekeeping gene, no such increase in signal of tumor over surrounding or vice versa is seen. These data taken together with the absence of Mig-7 expression in normal tissues (Northern data and RT-PCR, FIG. 9), show that Mig-7 can be used as a marker to detect migrating cancer cells in otherwise normal tissue. SF/cMet is known to cause an epithelial to mesenchyme morphology transition (Vande Woude et al., “Met-HGF/SF: Tumorigenesis, Invasion and Metastasis,” Ciba Foundation Symposium 212:119-130 (1997); Fafeur et al., “The ETS1 Transcription Factor is Expressed During Epithelial-Mesenchymal Transitions in the Chick Embryo and is Activated in Scatter Factor-Stimulated MDCK Epithelial Cells,” Cell Growth and Differentiation 8:655-665 (1997), which are hereby incorporated by reference in their entirety) therefore, it is difficult for pathologists to detect migrating transitioned cancer cells in the stroma surrounding the tumor. Detection of Mig-7 mRNA or protein can be used to detect these migrating cells in order to facilitate removal of cancer cells outside of the tumor during surgery. Other methods that can be used to detect Mig-7 expression includes, but is not limited to, in situ hybridization, RT-PCR, and immunohistochemistry.

TABLE 1
List of known information on samples of array in FIG. 10B (arrows) in which
Mig-7 expression was higher in the tumor than in the surrounding tissue.

| | Type of | | | Tumor | ICD | ICD |
|---|---|---|---|---|---|---|
| Sample | Cancer | Stage | Metastases | Size | Class | Morphology |
| N2 | Breast- | N/A | Lymph nodes | 2.5 cm | 174.9 | M8500/3 |
| | infiltrating | | | | | |
| | ductal | | | | | |
| Q2 | Breast- | 1 | N/A | N/A | N/A | N/A |
| | infiltrating | | | | | |
| | ductal | | | | | |
| CC2 | Breast- | N/A | N/A | N/A | N/A | N/A |
| | infiltrating | | | | | |
| | lobular | | | | | |
| L4 | Breast- | 2B | Yes | N/A | 174.9 | M8500/3 |
| | infiltrating | | | | | |
| | ductal | | | | | |
| J4 | Breast- | 3A | Yes | N/A | 174.9 | M8500/3 |
| | infiltrating | | | | | |
| | ductal | | | | | |
| R4 | Breast- | N/A | Lymph nodes | N/A | 174.9 | M8500/3 |
| | infiltrating | | | | | |
| | ductal | | | | | |
| T4 | Breast- | N/A | Adipose | N/A | 174.9 | M8480/3 |
| | mucinous | | | | | |
| | adenocarcinoma | | | | | |
| A8 | Uterus- | N/A | None seen | 5 × 0.5 cm | 182.0 | M8140/3 |
| | NOS | | | | | |
| | adenocarcinoma | | | | | |
| Y8 | Uterus- | 1B | N/A | N/A | 182.0 | M8140/3 |
| | NOS | | | | | |
| | adenocarcinoma | | | | | |
| F10 | Uterus- | N/A | None seen | N/A | 180.9 | M8071/3 |
| | keratinizing | | | | | |
| | squamous | | | | | |
| | cell | | | | | |
| L10 | Uterus- | N/A | None seen | N/A | 180.9 | M8070/3 |
| | NOS | | | | | |
| | squamous | | | | | |
| | cell | | | | | |
| A14 | Colon- | N/A | None seen | N/A | 154.1 | M8140/3 |
| | NOS | | | | | |
| | adenocarcinoma | | | | | |
| Z19 | Stomach- | N/A | N/A | N/A | 151.9 | M8140/3 |
| | NOS | | | | | |
| | adenocarcinoma | | | | | |
| D24 | Ovary- | 1 | None seen | N/A | 183.0 | M8980/3 |
| | NOS | | | | | |
| F28 | Lung- | 1 | N/A | N/A | 182.0 | M8240/3 |
| | malignant | | | | | |
| | carcinoid | | | | | |
| G28 | Lung- | N/A | N/A | N/A | N/A | N/A |
| | NOS | | | | | |
| | squamous | | | | | |
| | cell | | | | | |
| N28 | Lung- | 1 | N/A | N/A | 162.9 | M8140/3 |
| | NOS | | | | | |
| | adenocarcinoma | | | | | |
| U28 | Lung- | N/A | None seen | N/A | 162.9 | M8070/3 |
| | NOS | | | | | |
| | squamous | | | | | |
| | cell | | | | | |
| F32 | Kidney- | N/A | N/A | N/A | N/A | N/A |
| | transitional | | | | | |
| | cell | | | | | |
| P36 | Rectum- | N/A | None seen | N/A | 154.1 | M8140/3 |
| | NOS | | | | | |
| | adenocarcinoma | | | | | |
| Z19 | | | | | | |

TABLE 2
Samples in which Mig-7 expression was higher in surrounding tissue
than in tumor. Note that two out of four are advanced cancers (stage 3).

| | Cancer | | Meta- | Size of | ICD | ICD Mor- |
|---|---|---|---|---|---|---|
| Sample | Type | Stage | stases | Tumor | Class | phology |
| S3 | Breast- | N/A | Lymph | N/A | 174.9 | M8500/3 |
| | infiltrating | | nodes | | | |
| | ductal | | | | | |
| H13 | Colon- | 3 | N/A | N/A | N/A | N/A |
| | adenocarcinoma | | | | | |
| I13 | Colon- | 3 | N/A | N/A | N/A | N/A |
| | Colloid | | | | | |
| B9 | Uterus-NOS | 1B | N/A | N/A | 182.0 | M8140/3 |
| | Adenocarcinoma | | | | | |

### Example 23

### Method of Detecting Cancer Cells in the Blood of Human Patients

Following Internal Review Board approval for collection of human blood samples (#1869), RNA was isolated using BD TriReagent (Sigma), DNAsed the samples to eliminate contaminating genomic DNA, and RT-PCR was performed using the same primers and cycling parameters as described in FIG. 4B. Blood RNA was analyzed from five cancer patients and four normal individuals not diagnosed with cancer. As shown in FIG. 10C, two (one endometrial and one breast) out of five cancer patients (40%) were positive for Mig-7 expression, while none of the normal individuals were positive (FIG. 10C). The two Mig-7 positive patients had not had any radiation or chemotherapy only patient 5 fit that criteria and was not positive. The patients' samples in lanes 1 and 4 had both surgery and some follow up radiation or chemotherapy before the blood draw. As a result, Mig-7 expression can be used as a marker for routine physicals, initial cancer diagnoses, or determination of therapy efficacy by analyses of the patient's blood for presence of cancer cells. Compared to other blood assays for cancer cells using markers such as cytokeratin and mammaglobin, Mig-7 as a marker has a higher detection success rate (40% as compared to 8% for mammaglobin (Gurnewald et al., “Mammaglobin Gene Expression: A Superior Marker of Breast Cancer Cells in Peripheral Blood in Comparison to Epidermal-Growth-Factor Receptor and Cytokeratin-19,” Laboratory Investigation 80:1071-1077 (2000), which is hereby incorporated by reference in its entirety) and is far more specific than cytokeratin markers which are also found in normal epithelial cells (Burchill et al., “Detection of Epithelial Cancer Cells in Peripheral Blood by Reverse Transcriptase-Polymerase Chain Reaction,” British Journal of Cancer 71:278-281(1995), which is hereby incorporated by reference in its entirety). Method for Mig-7 expression in human blood samples includes Mig-7 specific antibodies as well as other activity, protein and RNA detection methods specific to Mig-7.

### Example 24

### Method of Mig-7 Regulation by the αvβ5 Integrin

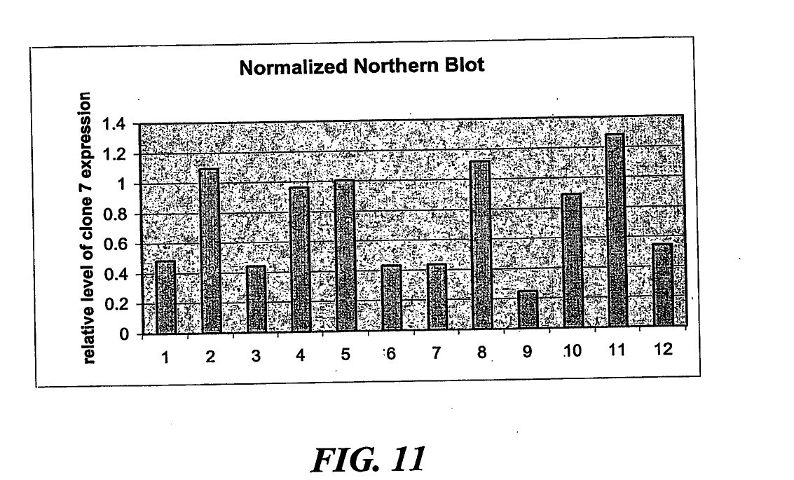

It was hypothesized that integrin expression and activation may also play a role in Mig-7 expression. This hypothesis was based on the presence of an RGD site in SF, a lack of SF-induction of Mig-7 expression in primary endometrial epithelial cells, previous reports that SF affects cells differently because of the differentiation state of the cell (Birchmeier et al., “Role of HGF/SF and c-Met in Morphogenesis and Metastasis of Epithelial Cells,” Ciba Foundation Symposium 212:230-246 (1997), which is hereby incorporated by reference in its entirety) and recent reports of direct interaction of non extracellular matrix proteins with integrins (Munger et al., “Interactions Between Growth Factors and Integrins: Latent Forms of Transforming Growth Factor-b are Ligands for the Integrin avb1,” Molecular Biology of the Cell 9:2627-2638 (1998); Andersen et al., “Bovine PAS-6/7 Binds Alpha v Beta 5 Integrins and Anionic Phospholipids Through Two Domains,” Biochemistry, 36:5441-5446 (1997); von Schlippe et al., “Functional Interaction Between E-Cadherin and av-Containing Integrins in Carcinoma Cells,” Journal of Cell Science 113:425-437 (2000), which are hereby incorporated by reference in their entirety). In addition, SF-induced migration in concert with integrin activation has been described before (Weimar et al., “Hepatocyte Growth Factor/Scatter Factor Promotes Adhesion of Lymphoma Cells to Extracellular Matrix Molecules Via Alpha 4 Beta 1 and Alpha 5 Beta 1 Integrins,” Blood 89:990-1000 (1997); Witzenbichler et al., “1999) Regulation of Smooth Muscle Cell Migration and Integrin Expression by the Gax Transcription Factor,” Journal of Clinical Investigation 104:1469-1480 (1999); Trusolino et al., “Growth Factor-Dependent Activation of avb3 Integrin in Normal Epithelial Cells: Implications for Tumor Invasion,” Journal of Cell Biology 142:1145-1156 (1998), which are hereby incorporated by reference in their entirety). Blocking antibodies to various integrins previously reported to be involved in cell migration have been chosen for use. The alphav beta5 integrin has been shown to be involved with glioma and squamous cell carcinoma invasion (Jones et al., “Changes in the Expression of Alpha v Integrins in Oral Squamous Cell Carcinomas,” Journal of Oral Pathology & Medicine 26:63-68 (1997); Tonn et al., “Invasive Behaviour of Human Gliomas is Mediated by Interindividually Different Integrin Patterns,” Anticancer Research 18:2599-2605 (1998), which are hereby incorporated by reference in their entirety). While alpha v beta 6 has been detected and shown to be activated in several types of carcinoma cells Breuss et al., “Expression of the b6 Integrin Subunit in Development, Neoplasia and Tissue Repair Suggests a Role in Epithelial Remodeling,” Journal of Cell Science 108:2241-2251 (1995), which is hereby incorporated by reference in its entirety). Integrin blocking antibodies were used after the cells had been plated and grown to 70% confluency, washed, incubated in serum- and phenol-free medium for 24 hours, then directly treated with the respective blocking antibody for 30 minutes at 37° C. incubation (5% CO₂). SF, at the indicated concentrations, was then added to the treated cultures for six hours. Total RNA was isolated and analyzed by Northern blot and densitometry (Bio-Rad Molecular Imager). The Northern blot was probed with a ³²P-labeled Mig-7 specific probe and then stripped and reprobed with actin. As can be seen in FIG. 11, blocking antibodies to αvβ5 integrins also blocked the normal six-hour SF induction of Mig-7 (compare lane 6 with lane 7). Adding additional SF rescued Mig-7 expression (lane 8). In contrast, treatment with blocking antibodies to β1 or to αvβ6 integrins did not block the six hour SF induction of Mig-7 even though αvβ6 integrins bind to vitronectin as do alphav beta5 integrins (Huang et al., “The Integrin αvβ5 is Critical for Keratinocyte Migration of Both its Known Ligand, Fibronectin, and on Vitronectin,” Journal of Cell Science 111:2189-2195 (1998), which is hereby incorporated by reference in its entirety). Alphav beta5 integrins are found on 17 oral squamous cell carcinomas (Jones et al., “Changes in the Expression of Alpha v Integrins in Oral Squamous Cell Carcinomas,” Journal of Oral Pathology & Medicine 26:63-68 (1997), which is hereby incorporated by reference in its entirety), a type of metastatic cancer that we found to express Mig-7 (FIGS. 10A-10C). Also, blocking antibodies to alphav beta5 integrin inhibits invasion of human gliomas into rat brain aggregates (Tonn et al., “Invasive Behaviour of Human Gliomas is Mediated by Interindividually Different Integrin Patterns,” Anticancer Research 18:2599-2605 (1998), which is hereby incorporated by reference in its entirety). These results imply specific cross talk between c-Met and αvβ5 integrins signal transduction. In addition, these data imply that specific integrin expression is required for Mig-7 expression and may be required for migration of RL-95 cells.

### Example 25

### Method for Inhibiting Cancer Cell Migration

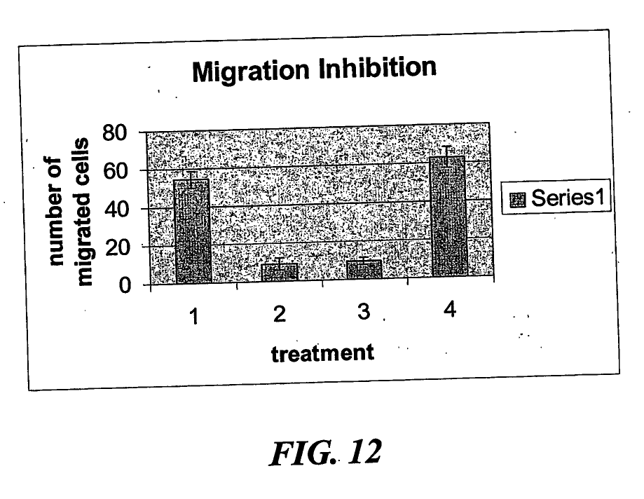

Using antisense oligonucleotides designed to the region surrounding the Mig-7 Kozak sequence, RL-95 cell migration was inhibited in a wound healing assay in vitro. The antisense olignucleotide sequences are: 5′ GCA CTA TGG GCT TAT GGG 3′ (SEQ ID NO:40) (antisense to nucleotides 275-292 of SEQ ID NO:1 or antisense to nucleotides 760-777 of SEQ ID NO:2), and 5′GCA TCT ACT TGC TGC CAT GG 3′ (SEQ ID NO:41) (antisense to nucleotides 324-343 of SEQ ID NO:1 or antisense to nucleotides 809-828 of SEQ ID NO:2). This inhibition was not seen using an irrelevant oligonucleotide (sequence 5′GGG TAT TCG GGT ATT ACG 3′ (SEQ ID NO:42)). This experiment was performed in triplicate wells. All wells were treated with SF. Three fields of view were counted in the scraped “wound” area per well then averaged. The results are shown in FIG. 12. As shown, only the antisense oligos (2 and 3) and not the irrelevant oligo (4) inhibited migration six-fold when compared to cells treated with no oligo (1). These data suggest that Mig-7 plays a role in regulating migration of RL-95 endometrial carcinoma cells. This experiment has been repeated twice showing the same inhibition in migration. Other methods for inhibiting cancer cell migration using Mig-7 expression as a target include but are not limited to ribozymes, small molecules that block Mig-7 expression, proteins that enhance Mig-7 mRNA secondary structure that inhibit translation, blocking antibodies, and others.

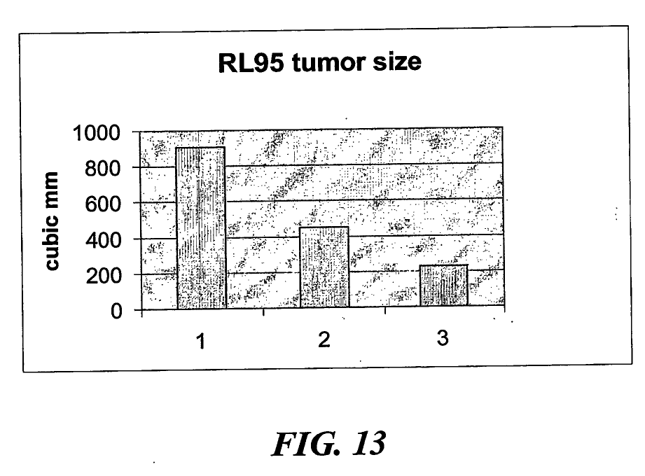

Mig-7 specific antisense oligonucleotide inhibition of RL-95 cells was also observed in vivo. Cells were treated in vitro as before in serum free phenol free media for two days then treated with respective oligonucleotides followed by trypsinization to remove from the culture plate and SF treatment. One hundred thousand cells were added to 500 μl of Matrigel (low growth factor form, BD Biosciences) and then injected subcutaneously at the dorsal neck of nude mice. As shown in FIG. 13, the size of primary tumor was greater in the Mig-7 antisense oligonucleotide due to a lack of migration. The tumor size of control oligo and of no oligo treated animal tumors were 2- and 4-fold less respectively than Mig-7 antisense treated tumors showing that more migration from the site of injection occurred with the control and no oligo treated cells. Other means of treatment may include periodic infusion of antisense or other Mig-7 specific reagents mentioned previously either at the site of primary tumor, systemically, or localized.

### Example 26

### Insulin Like Growth Factor and Epidermal Growth Factor Upregulate Mig-7 in αvβ5 Positive Pancreatic Carcinoma Cells

Accumulating evidence suggests that αvβ5 interacts with the SF/Met/Mig-7 pathway. αvβ5 integrins were found on 17 oral squamous cell carcinomas (Jones et al., “Changes In the Expression of Alpha v Integrins in Oral Squamous Cell Carcinomas,” Journal of Oral Pathology & Medicine 26:63-68 (1997), which is hereby incorporated by reference in its entirety), which is the same type of metastatic cancer that was found to express Mig-7 (FIGS. 3A & 3B). Blocking antibodies to αvβ5 inhibited invasion of human gliomas into rat brain aggregates (Tonn et al., “Invasive Behaviour of Human Gliomas Is Mediated by Interindividually Different Integrin Patterns,” Anticancer Research 18:2599-2605 (1998), which is hereby incorporated by reference in its entirety). More importantly, αvβ5 has been reported to play a role in activation-dependent cell migration (Klemke et al., “Receptor Tyrosine Kinase Signaling Required for Integrin Alpha v Beta 5-Directed Cell Motility But Not Adhesion on Vitronectin,” Journal of Cell Biology 127:859-866 (1994), which is hereby incorporated by reference in its entirety). In CS-1 melanoma, MCF-7PB breast carcinoma, and FG pancreatic carcinoma cells that express αvβ5 but not αvβ3, binding of both insulin-like growth factor receptor and αvβ5 is required for spontaneous pulmonary metastasis but is not required for primary tumor growth (Brooks et al., “Insulin-Like Growth Factor Receptor Cooperates With Integrin Alpha v Beta 5 to Promote Tumor Cell Dissemination In Vivo,” Journal of Clinical Investigation 99:1390-1398 (1997), which is hereby incorporated by reference in its entirety). Indeed, Mig-7 is induced in αvβ5 positive FG pancreatic cells (generously provided by Dr. David Cheresh, The Scripps Institute) using the same culture conditions as previously described for SF-induced Mig-7 expression in RL95 and HEC1A cells. Klemke et al. have provided evidence that the EGF receptor EGFR) when bound by its ligand induces αvβ5-dependent cell migration on vitronectin (Klemke et al., “Receptor Tyrosine Kinase Signaling Required for Integrin Alpha v Beta 5-Directed Cell Motility But Not Adhesion on Vitronectin,” Journal of Cell Biology 127:859-866 (1994), which is hereby incorporated by reference in its entirety). The ILGFR has also been shown to cooperate with the αvβ5 integrin to promote tumor metastasis (Brooks et al., “Insulin-Like Growth Factor Receptor Cooperates With Integrin Alpha v Beta 5 to Promote Tumor Cell Dissemination In Vivo,” Journal of Clinical Investigation 99:1390-1398 (1997), which is hereby incorporated by reference in its entirety). However, the genes expressed during this crosstalk have not been determined until the present invention.

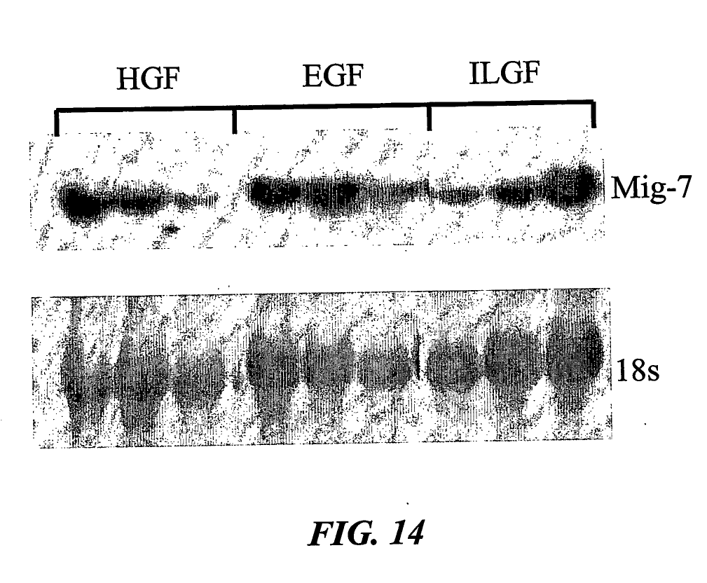

FG cells were treated with 20 ng/ml ILGF or 100 ng/ml EGF after culturing in RPMI-1640 supplemented with 10% fetal bovine serum, 2 mM L-glutanine, and 50 g/ml gentamicin for four days and serum starvation for 48 hours as described previously. Mig-7 expression in this FG cell line as a result of HGF, EGF, or ILGF treatment (FIG. 14) suggests that Mig-7 is involved in the same tyrosine kinase receptor signaling cascade that requires αvβ5 signaling as described by Klemke et al. (Klemke et al., “Receptor Tyrosine Kinase Signaling Required for Integrin Alpha v Beta 5-Directed Cell Motility But Not Adhesion on Vitronectin,” Journal of Cell Biology 127:859-866 (1994), which is hereby incorporated by reference in its entirety). Thus, Mig-7 is the first gene expression identified during this signaling.

The observation that HGF does not induce Mig-7 expression in primary endometrial epithelial cells even though these cells express the HGF receptor (FIG. 4B), suggests that the Met signaling pathway bifurcates. In carcinoma cells, the Mig-7 induction pathway is activated, while in normal cells, this pathway is suppressed. It is thus likely that cross-talk between the HGF/Met and other ligands of tyrosine kinase receptors, including EGF and ILGF pathways determine whether or not Mig-7 induction occurs. Integrin signal transduction pathways are regulated by the state of cell differentiation. Since carcinoma cells are typically in a less differentiated state than normal cells, it is not surprising that marked differences exist in integrin signaling between normal and cancer cells (Giancotti et al., “Integrin Signaling,” Science 285:1028-1032 (1999), which is hereby incorporated by reference in its entirety). These results suggest specific cross talk exists between tyrosine kinase receptors and αvβ5 signal transduction pathways. In addition, these data suggest that αvβ5 integrin expression is required for SF-HGF- or ILGF-induced Mig-7 expression.

### Example 27

### Expression of Mig-7 in Early Placenta

Mig-7 has been shown to express in early placenta (FIG. 15). HGF is required for placental formation (Uehara et al., “Placental Defect and Embryonic Lethality in Mice Lacking Hepatocyte Growth Factor/Scatter Factor, Nature 373:702-705 (1995), which is hereby incorporated by reference in its entirety). Cytotrophoblasts that establish placental anchoring villi require HGF for migration (Dokras et al., “Regulation of Human Cytotrophoblast Morphogenesis Hepatocyte Growth Factor/Scatter Factor,” Biology of Reproduction 65(4):1278-1288 (2001); Zhou et al., “Human Cytotrophoblasts Adopt a Vascular Phenotype as They Differentiate,” Journal of Clinical Investigation 99(9):2139-2151 (1997), which are hereby incorporated by reference in their entirety), possess αvβ5 integrins and invade maternal blood vessels similar to carcinoma cells (Zhou et al., “Human Cytotrophoblasts Adopt a Vascular Phenotype as They Differentiate,” Journal of Clinical Investigation 99(9):2139-2151 (1997), which is hereby incorporated by reference in its entirety). A lack of this invasion leads to smaller than gestational age (SGA) growth of fetus and increased risk for preeclampsia and eclampsia (Dokras et al., “Regulation of Human Cytotrophoblast Morphogenesis Hepatocyte Growth Factor/Scatter Factor,” Biology of Reproduction 65(4):1278-1288 (2001); Zhou et al., “Human Cytotrophoblasts Adopt a Vascular Phenotype as They Differentiate,” Journal of Clinical Investigation 99(9):2139-2151 (1997), which are hereby incorporated by reference in their entirety). Thus, since Mig-7 is expressed in early placenta, the only stage at which cytotrophoblast cells migrate and invade (Dokras et al., “Regulation of Human Cytotrophoblast Morphogenesis Hepatocyte Growth Factor/Scatter Factor,” Biology of Reproduction 65(4):1278-1288 (2001); Zhou et al., “Human Cytotrophoblasts Adopt a Vascular Phenotype as They Differentiate,” Journal of Clinical Investigation 99(9):2139-2151 (1997), which are hereby incorporated by reference in their entirety), enhancing Mig-7 expression may prevent SGA growth, preeclampsia and eclampsia.

In addition, fetal cytotrophoblast cells after invading the maternal blood supply can cause increased risk for immune disease (Tanaka et al., “Fetal Microchimerisms in the Mother: Immunologic Implications,” Liver Transplantation 6(2):138-43 (2000), which is hereby incorporated by reference in its entirety). Therefore, inhibition of Mig-7 expression may block these fetal cells from invading the maternal blood supply and decrease this risk of immune disease.

Fetal cells that have invaded the maternal blood supply can also be used for diagnostic purposes (Pertl et al., “First Trimester Prenatal Diagnosis: Fetal Cells in the Maternal Circulation,” Seminars in Perinatology 23(5):393402 (1999), which is hereby incorporated by reference in its entirety). Accordingly, Mig-7 may be used as a target to isolate these cells.

Although preferred embodiments have been depicted and described in detail herein, it will be apparent to those skilled in the relevant art that various alternatives, modifications, variations, additions, substitutions, improvements, substantial equivalents, and the like can be made without departing from the spirit of the invention and these are therefore considered to be within the scope of the invention as defined in the claims which follow.

1. An isolated nucleic acid molecule conferring on a mammalian carcinoma cell an ability to undergo cell migration.

2. The isolated nucleic acid molecule according to claim 1, wherein expression of the nucleic acid molecule is induced in vivo by a growth factor selected from the group consisting of hepatocyte growth factor, insulin like growth factor, and epidermal growth factor.

3. The isolated nucleic acid molecule according to claim 1, wherein expression of the nucleic acid molecule is induced in vivo by activation of a tyrosine kinase protooncongene receptor.

4. The isolated nucleic acid molecule according to claim 4, wherein said tyrosine kinase protooncongene receptor is c-Met, insulin receptor, insulin like growth factor receptor, epidermal growth factor receptors, and platelet derived growth factor receptor.

5. The isolated nucleic acid molecule according to claim 2, wherein expression of the nucleic acid molecule is induced in vivo by activation of integrin αvβ5 or integrin αvβ3.

6. The isolated nucleic acid molecule according to claim 1 wherein the nucleic acid molecule has a nucleotide sequence selected from the group consisting of SEQ ID NO:1 and SEQ ID NO:2.

7. The isolated nucleic acid molecule according to claim 1, wherein the nucleic acid sequence is 99% homologous to either SEQ ID NO:1 or SEQ ID NO:2.

8. The isolated nucleic acid molecule according to claim 1, wherein the nucleic acid molecule encodes a protein or polypeptide comprising an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

9. The isolated nucleic acid molecule according to claim 1, wherein the nucleic acid molecule has a nucleotide sequence comprising at least 18 contiguous nucleic acid residues that hybridize to either SEQ ID NO:1 or SEQ ID NO:2 under stringent conditions selected from the group consisting of: (a) 6×SSC at 68° C.; (b) 5×SSC and 50% formamide 37° C.; and (c) 2×SSC and 40% formamide at 40° C.

10. The isolated nucleic acid molecule according to claim 1, wherein said migratory gene encodes a protein or polypeptide having a molecular weight of about 20 to 40 kilodaltons.

11. The isolated nucleic acid molecule according to claim 1, wherein said isolated nucleic acid molecule confers on a human carcinoma cell an ability to undergo cell migration.

12. The isolated nucleic acid molecule according to claim 11, wherein said human carcinoma cell is selected from the group consisting of an ovary cell, a colon cell, an endometrial cell, a squamous cell, a uterus cell, a stomach cell, a lung cell, a breast cell, a prostate cell, a kidney cell, a rectum cell, a thyroid cell, a pancreas cell, a cervix cell, and intestine cell.

13. A recombinant DNA expression system comprising an expression vector into which is inserted an isolated nucleic acid molecule according to claim 1.

14. The recombinant DNA expression system according to claim 13, wherein said nucleic acid molecule is heterologous to the expression vector.

15. The recombinant DNA expression system according to claim 14, wherein said nucleic acid molecule is inserted into said vector in proper sense orientation and correct reading frame.

16. A host cell incorporating an isolated nucleic acid molecule according to claim 1.

17. The host cell according to claim 16, wherein said isolated nucleic acid molecule is heterologous to the host cell.

18. An antisense oligonucleotide comprising at least 8 contiguous nucleic acid residues targeted to a nucleic acid molecule conferring on a mammalian carcinoma cell an ability to undergo cell migration.

19. The antisense oligonucleotide according to claim 18, wherein expression of the nucleic acid molecule is induced in vivo by a hepatocyte growth factor.

20. The antisense oligonucleotide according to claim 18, wherein expression of the nucleic acid molecule is induced in vivo by activation of a tyrosine kinase protooncongene receptor.

21. The antisense oligonucleotide according to claim 20, wherein said tyrosine kinase protooncongene receptor is c-Met.

22. The antisense oligonucleotide according to claim 18, wherein expression of the nucleic acid molecule is induced in vivo by activation of integrin αvβ5.

23. The antisense oligonucleotide according to claim 18, wherein said nucleic acid molecule has a nucleotide sequence selected from the group consisting of SEQ ID NO:1 and SEQ ID NO:2.

24. The antisense oligonucleotide according to claim 18, wherein said antisense oligonucleotide hybridizes to said nucleic acid molecule, or to a complementary sequence of said nucleic acid molecule, under stringent conditions selected from the group consisting of: (a) 6×SSC at 68° C.; (b) 5×SSC and 50% formamide 37° C.; and (c) 2×SSC and 40% formamide at 40° C.

25. The antisense oligonucleotide according to claim 18, wherein said antisense oligonucleotide hybridizes to a nucleic acid molecule comprising nucleotides 275 to 292 of SEQ ID NO:1 or comprising nucleotides 324 to 343 of SEQ ID NO:1.

26. The antisense oligonucleotide according to claim 18, wherein said antisense oligonucleotide hybridizes to a nucleic acid molecule comprising nucleotides 760 to 777 of SEQ ID NO:2 or comprising nucleotides 809 to 828 of SEQ ID NO:2.

27. A method for inhibiting expression, in a subject, of a nucleic acid molecule conferring on a human carcinoma cell an ability to undergo cell migration, said method comprising:
administering to said subject an inhibitor capable of blocking binding of a growth factor to at least one receptor for the growth factor under conditions effective to inhibit the expression of the nucleic acid molecule.

28. The method according to claim 27, wherein said growth factor is selected from the group consisting of hepatocyte growth factor, insulin like growth factor, and epidermal growth factor, and wherein said receptor is selected from the group consisting of c-Met, insulin receptor, insulin like growth factor receptor, epidermal growth factor receptors, platelet derived growth factor receptor, integrin αvβ5, and integrin αvβ3.

29. The method according to claim 27, wherein said inhibitor binds to the hepatocyte growth factor.

30. The method according to claim 27, wherein said inhibitor binds to the receptor.

31. A method for inhibiting production, in a subject, of a protein or polypeptide encoded by a nucleic acid molecule conferring on a carcinoma cell an ability to undergo cell migration, said method comprising:
administering to said subject an antisense oligonucleotide complementary to a target portion of the nucleic acid molecule under conditions effective to inhibit production of the protein or polypeptide.

32. The method according to claim 31, wherein the nucleic acid molecule has a nucleotide sequence selected from the group consisting of SEQ ID NO:1 and SEQ ID NO:2.

33. The method according to claim 31, wherein said antisense oligonucleotide hybridizes to said nucleic acid molecule, or to a complementary sequence of said nucleic acid molecule, under stringent conditions selected from the group consisting of: (a) 6×SSC at 68° C.; (b) 5×SSC and 50% formamide 37° C.; and (c) 2×SSC and 40% formamide at 40° C.

34. The method according to claim 31, wherein said antisense oligonucleotide hybridizes to a nucleic acid molecule comprising nucleotides 275 to 292 of SEQ ID NO:1 or comprising nucleotides 324 to 343 of SEQ ID NO:1.

35. The method according to claim 31, wherein said antisense oligonucleotide hybridizes to a nucleic acid molecule comprising nucleotides 760 to 777 of SEQ ID NO:2 or comprising nucleotides 809 to 828 of SEQ ID NO:2.

36. A method for inhibiting metastasis of a carcinoma cell in a subject, said method comprising:
administering to said subject an antisense oligonucleotide complementary to a target portion of a nucleic acid molecule conferring on a carcinoma cell an ability, in vivo, to undergo cell migration under conditions effective to inhibit metastasis of the carcinoma cell.

37. The method according to claim 36, wherein the nucleic acid molecule has a nucleotide sequence selected from the group consisting of SEQ ID NO:1 and SEQ ID NO:2.

38. The method according to claim 36, wherein said antisense oligonucleotide hybridizes to said nucleic acid molecule, or to a complementary sequence of said nucleic acid molecule, under stringent conditions selected from the group consisting of: (a) 6×SSC at 68° C.; (b) 5×SSC and 50% formamide 37° C.; and (c) 2×SSC and 40% formamide at 40° C., wherein said antisense oligonucleotide inhibits the production of a protein or polypeptide encoded by said nucleic acid molecule.

39. The method according to claim 36, wherein said antisense oligonucleotide hybridizes to a nucleic acid molecule comprising nucleotides 275 to 292 of SEQ ID NO:1 or comprising nucleotides 324 to 343 of SEQ ID NO:1.

40. The method according to claim 36, wherein said antisense oligonucleotide hybridizes to a nucleic acid molecule comprising nucleotides 760 to 777 of SEQ ID NO:2 or comprising nucleotides 809 to 828 of SEQ ID NO:2.

41. A method for inhibiting metastasis of a carcinoma cell in a human subject, said method comprising:
administering to said subject an inhibitor capable of blocking the binding of a growth factor to at least one receptor for the growth factor under conditions effective to inhibit metastasis of said carcinoma cell.

42. The method according to claim 41, wherein said growth factor is selected from the group consisting of hepatocyte growth factor, insulin like growth factor, and epidermal growth factor, and wherein said receptor is selected from the group consisting of c-Met, insulin receptor, insulin like growth factor receptor, epidermal growth factor receptors, platelet derived growth factor receptor, integrin αvβ5, and integrin αvβ3.

43. The method according to claim 41, wherein said inhibitor binds to the hepatocyte growth factor.

44. The method according to claim 41, wherein said inhibitor binds to the receptor.

45. A method for inhibiting migration of a carcinoma cell in a subject, said method comprising:
administering to said subject an antisense oligonucleotide complementary to a target portion of a nucleic acid molecule conferring on a carcinoma cell an ability, in vivo, to undergo cell migration under conditions effective to inhibit migration of said carcinoma cell.

46. The method according to claim 45, wherein the nucleic acid molecule has a nucleotide sequence selected from the group consisting of SEQ ID NO:1 and SEQ ID NO:2.

47. The method according to claim 45, wherein said antisense oligonucleotide hybridizes to said nucleic acid molecule, or to a complementary sequence of said nucleic acid molecule, under stringent conditions selected from the group consisting of: (a) 6×SSC at 68° C.; (b) 5×SSC and 50% formamide 37° C.; and (c) 2×SSC and 40% formamide at 40° C.

48. The method according to claim 45, wherein said antisense oligonucleotide hybridizes to a nucleic acid molecule comprising nucleotides 275 to 292 of SEQ ID NO:1 or comprising nucleotides 324 to 343 of SEQ ID NO:1.

49. The method according to claim 45, wherein said antisense oligonucleotide hybridizes to a nucleic acid molecule comprising nucleotides 760 to 777 of SEQ ID NO:2 or comprising nucleotides 809 to 828 of SEQ ID NO:2.

50. A method for inhibiting migration of a carcinoma cell in a subject, said method comprising:
administering to said subject an inhibitor capable of blocking binding of a growth factor to at least one receptor for the growth factor under conditions effective to inhibit migration of said carcinoma cell.

51. The method according to claim 50, wherein said growth factor is selected from the group consisting of hepatocyte growth factor, insulin like growth factor, and epidermal growth factor, and wherein said receptor is selected from the group consisting of c-Met, insulin receptor, insulin like growth factor receptor, epidermal growth factor receptors, platelet derived growth factor receptor, integrin αvβ5, and integrin αvβ3.

52. The method according to claim 50, wherein said inhibitor binds to the hepatocyte growth factor.

53. The method according to claim 50, wherein said inhibitor binds to the receptor.

54. A protein or polypeptide encoded by the nucleic acid molecule according to claim 1.

55. A protein or polypeptide according to claim 54, wherein the protein or polypeptide comprises an amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ BD NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

56. An isolated antibody or binding portion thereof raised against a protein or polypeptide according to claim 54.

57. The isolated antibody or binding portion thereof according to claim 56, wherein said antibody is monoclonal or polyclonal.

58. A method for detecting the presence of a migrating carcinoma cell in a sample of a subject's tissue or body fluids comprising:
providing a protein or polypeptide according to claim 56 as an antigen;
contacting the sample with the antigen; and
detecting any reaction which indicates that the migrating carcinoma cell is present in the sample using an assay system.

59. The method according to claim 58, wherein the assay system is selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

60. A method for detecting the presence of a migrating carcinoma cell in a sample of a subject's tissue or body fluids comprising:
providing an antibody or binding portion thereof according to claim 56;
contacting the sample with the antibody or binding portion thereof; and
detecting any reaction which indicates that the migrating carcinoma cell is present in the sample using an assay system.

61. The method according to claim 60, wherein the assay system is selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

62. A method for detecting the presence of a migrating carcinoma cell in a sample of a subject's tissue or body fluids comprising:
providing a nucleotide sequence of the nucleic acid molecule according to claim 1 as a probe in a nucleic acid hybridization assay;
contacting the sample with the probe; and
detecting any reaction which indicates that the migrating carcinoma cell is present in the sample.

63. A method for detecting the presence of a migrating carcinoma cell in a sample of a subject's tissue or body fluids comprising:
providing a nucleotide sequence of the nucleic acid molecule according to claim 1 as a probe in a gene amplification detection procedure;
contacting the sample with the probe; and
detecting any reaction which indicates that the migrating carcinoma cell is present in the sample.

64. A method of inhibiting the migration of placental cells into a blood stream of a mammalian subject, said method comprising:
administering to said mammalian subject an inhibitor capable of blocking binding of a growth factor to at least one receptor for the growth factor under conditions effective to inhibit migration of said placental cells.

65. The method according to claim 64, wherein the placental cells are cytotrophoblast cells.

66. A method of inhibiting the migration of placental cells into a blood stream of a mammalian subject, said method comprising:
administering to said mammalian subject an antisense oligonucleotide complementary to a target portion of a nucleic acid molecule conferring on the placental cells an ability, in vivo, to undergo cell migration under conditions effective to inhibit migration of said placentals cells into the blood stream.

67. The method according to claim 66, wherein the placental cells are cytotrophoblast cells.

68. A method of inducing the establishment of anchoring villi and blood supply to a mammalian fetus, said method comprising:
transducing ectopic expression of the nucleic acid molecule according to claim 1 using a suitable expression vector into cytotrophoblast cells or precursors thereof,
under conditions effective to induce the establishment of anchoring villi and blood supply to a mammalian fetus.

69. A method of transgenically expressing the nucleic acid molecule according to claim 1 in a mammalian cell, said method comprising:
cloning the nucleic acid molecule according to claim 1 into a suitable expression vector and
transfecting said vector into a mammalian cell using suitable means of transfection selected from the group consisting of electroporation, lipophilic reagent, and calcium chloride,
under conditions effective to transgenically express the nucleic acid molecule in a mammalian cell.

70. A method for detecting the presence of fetal cytotrophoblast cells in a sample of a subject's tissue or body fluids, said method comprising:
providing a nucleotide sequence of the nucleic acid molecule according to claim 1 as a probe in a detection assay;
contacting the sample with the probe; and
detecting any reaction which indicates that fetal cytotrophoblast cells are present in the sample.

71. The method according to claim 70, wherein said detection assay is selected from the group consisting of a nucleic acid hybridization assay and a gene amplification detection procedure.
